(19) United States
(12) Patent Application Publication
(10) Pub. No.: US 2023/0343416 A1
(43) Pub. Date: Oct. 26, 2023

(54) METHODS AND SYSTEMS FOR SEQUENCE AND VARIANT CALLING

(71) Applicant: Ultima Genomics, Inc., Newark, CA (US)

(72) Inventors: Yoav ETZIONI, Tel Aviv (IL); Omer BARAD, Mazkeret Batya (IL); Avishai BARTOV, Newark, CA (US); Ilya SOIFER, Newark, CA (US); Ehud AMITAI, Tel Aviv (IL); Asaf HALLE, Petah Tikva (IL)

(21) Appl. No.: 18/025,603

(22) PCT Filed: Sep. 10, 2021
(86) PCT No.: PCT/US2021/049923

§ 371 Date: Mar. 9, 2023

Related U.S. Application Data
(60) Provisional application No. 63/076,820, filed on Sep. 10, 2020.

(51) Int. Cl. G16B 40/10; C12Q 1/6869; G16B 40/20; G16B 30/10; G06N 3/0464

(52) U.S. Cl. CPC G16B 40/10; C12Q 1/6869; G16B 40/20; G16B 30/10; G06N 3/0464

(57) ABSTRACT

The present disclosure provides methods, systems, and media for accurate and efficient estimation of a genome of a genus. The methods and systems described herein may be used to accurately determine a base sequence of a polynucleotide. Additionally, the methods and systems may be used to identify base variants of a polynucleotide.

### CROSS-REFERENCE

This application claims the benefit of U.S. Patent Application No. 63/076,820, filed Sep. 10, 2010, which is incorporated by reference herein in its entirety.

### BACKGROUND

The goal to elucidate the entire human genome has created interest in technologies for rapid nucleic acid (e.g., DNA) sequencing, both for small and large scale applications. As knowledge of the genetic basis for human diseases increases, high-throughput DNA sequencing has been leveraged for myriad clinical applications. Despite the prevalence of nucleic acid sequencing methods and systems in a wide range of molecular biology and diagnostics applications, such methods and systems may encounter challenges in accurate base calling, such as when sequencing signals include regions of repeating nucleotide bases called homopolymers. In particular, sequencing methods that perform base calling based on quantified characteristic signals indicating nucleotide incorporation can have sequencing errors (e.g., in quantifying homopolymer lengths), stemming from random and unpredictable systematic variations in signal levels and context dependent signals that may be different for every sequence. Such signal variations and context dependency signals may cause issues with sequence (e.g., homopolymer) calling.

### SUMMARY

Recognized herein is a need for improved base calling of sequences, such as sequences containing homopolymers. Methods and systems provided herein can significantly reduce or eliminate errors in base calling (e.g., related to quantifying homopolymer lengths and errors associated with context dependence). Such methods and systems may achieve accurate and efficient base calling of sequences (such as sequences containing homopolymers), quantification of homopolymer lengths, and quantification of context dependency in sequence signals.

In an aspect, the present disclosure provides a method for determining a sequence of a nucleic acid, comprising: (a) receiving a plurality of sequencing signals of the nucleic acid that are generated at least in part by imaging a substrate comprising a plurality of substrate segments; (b) applying a trained algorithm to at least a portion of the plurality of sequencing signals to estimate a likelihood that one or more of the plurality of sequencing signals is produced by a particular nucleic acid sequence; and (c) determining the sequence of the nucleic acid based at least in part on the estimated likelihoods from (b).

In some embodiments, the nucleic acid comprises deoxyribonucleic acid (DNA) or ribonucleic acid (RNA). In some embodiments, the plurality of sequencing signals are generated at least in part by performing flow sequencing of the nucleic acid. In some embodiments, the plurality of sequencing signals comprise analog values produced by the imaging. In some embodiments, the analog values comprise fluorescence signals. In some embodiments, the fluorescence signals correspond to discrete DNA extensions sensed from introduction of single nucleotide solutions in the flow sequencing. In some embodiments, the introduction of single nucleotide solutions in the flow sequencing is cyclic. In some embodiments, the introduction of single nucleotide solutions in the flow sequencing is acyclic. In some embodiments, the plurality of substrate segments are determined based on expected or actual differences between an illumination of the plurality of substrate segments. In some embodiments, the plurality of substrate segments are determined based on expected or actual differences between a collection or measurement of radiation from the plurality of substrate segments. In some embodiments, the plurality of substrate segments are determined based on expected or actual distributions of chemical materials over the plurality of substrate segments. In some embodiments, the plurality of substrate segments comprise a same shape and/or size. In some embodiments, at least two of the plurality of substrate segments differ by at least one shape and size.

In some embodiments, (b) further comprises estimating a likelihood of each of a plurality of haplotypes, and wherein (c) further comprises determining the sequence of the nucleic acid based at least in part on the estimated likelihoods of each of the plurality of haplotypes.

In some embodiments, the trained algorithm comprises a trained machine learning algorithm. In some embodiments, the trained machine learning algorithm comprises a neural network, a support vector machine, a random forest, or a deep learning algorithm. In some embodiments, the trained machine learning algorithm comprises the neural network. In some embodiments, the neural network comprises a convolutional neural network. In some embodiments, the trained algorithm is trained at least in part by: obtaining a training set comprising a plurality of training sequencing signals and a plurality of training sequencing reads associated therewith, and using the training set to generate the trained algorithm, wherein the trained algorithm comprises a mapping between input sequencing signals and output sequencing reads comprising base calls.

In some embodiments, training sequencing reads in the plurality of training sequencing reads are aligned to a reference genome. In some embodiments, the aligning is performed in flow space. In some embodiments, the aligning comprises using a set of common base calling variants. In some embodiments, the aligning comprises detecting contamination from a different genome. In some embodiments, the aligning comprises using indicators of pre-determined adapter sequences. In some embodiments, the plurality of training sequencing reads is filtered to remove at least one training sequencing read that is not fully aligned to the reference. In some embodiments, the plurality of training sequencing reads is filtered to remove at least one training sequencing read that does not comprise a largest segment that is fully aligned to the reference. In some embodiments, the plurality of training sequencing reads is filtered to remove at least one training sequencing read that has a quality score that fails to meet a pre-determined criterion. In some embodiments, the plurality of training sequencing reads is filtered to remove at least one training sequencing read that has a length that differs from a reference length. In some embodiments, the reference length is a most common length of the plurality of training sequencing reads. In some embodiments, the plurality of training sequencing reads is filtered to remove at least one training sequencing read that comprises a pre-determined adapter sequence. In some embodiments, at least one of the plurality of training sequencing reads is padded with filler values such that the plurality of training sequencing reads has a substantially identical length. In some embodiments, the filler values are masking values comprising negative numbers. In some embodiments, the negative numbers are indicative of a class of trimmed flows. In some embodiments, the class of trimmed flows is selected from the group consisting of low quality flows, flows comprising three consecutive zero-signals, flows with errors, and flows with variants. In some embodiments, the method further comprises determining a likelihood of the sequence of the nucleic acid determined in (c) being correct. In some embodiments, the method further comprises determining a maximum likelihood h-mer length of the sequence of the nucleic acid.

In another aspect, the present disclosure provides a method for determining a sequence of a nucleic acid, comprising: (a) receiving a plurality of sequencing signals of the nucleic acid that belong to at least one image of at least one part of a substrate that is linked to multiple DNA beads; (b) applying a trained algorithm to at least a portion of the plurality of sequencing signals to estimate a likelihood that one or more of the plurality of sequencing signals is produced by a particular nucleic acid sequence; and (c) determining the sequence of the nucleic acid based at least in part on the estimated likelihoods from (b).

In some embodiments, the nucleic acid comprises deoxyribonucleic acid (DNA) or ribonucleic acid (RNA). In some embodiments, the plurality of sequencing signals are generated at least in part by performing flow sequencing of the nucleic acid. In some embodiments, the plurality of sequencing signals comprise analog values produced by the imaging. In some embodiments, the analog values comprise fluorescence signals. In some embodiments, the fluorescence signals correspond to discrete DNA extensions sensed from introduction of single nucleotide solutions in the flow sequencing. In some embodiments, the introduction of single nucleotide solutions in the flow sequencing is cyclic. In some embodiments, the introduction of single nucleotide solutions in the flow sequencing is acyclic.

In some embodiments, (b) further comprises estimating a likelihood of each of a plurality of haplotypes, and wherein (c) further comprises determining the sequence of the nucleic acid based at least in part on the estimated likelihoods of each of the plurality of haplotypes.

In some embodiments, the trained algorithm comprises a trained machine learning algorithm. In some embodiments, the trained machine learning algorithm comprises a neural network, a support vector machine, a random forest, or a deep learning algorithm. In some embodiments, the trained machine learning algorithm comprises the neural network. In some embodiments, the neural network comprises a convolutional neural network. In some embodiments, the trained algorithm is trained at least in part by: obtaining a training set comprising a plurality of training sequencing signals and a plurality of training sequencing reads associated therewith, and using the training set to generate the trained algorithm, wherein the trained algorithm comprises a mapping between input sequencing signals and output sequencing reads comprising base calls.

In some embodiments, training sequencing reads in the plurality of training sequencing reads are aligned to a reference genome. In some embodiments, the aligning is performed in flow space. In some embodiments, the aligning comprises using a set of common base calling variants. In some embodiments, the aligning comprises detecting contamination from a different genome. In some embodiments, the aligning comprises using indicators of pre-determined adapter sequences. In some embodiments, the plurality of training sequencing reads is filtered to remove at least one training sequencing read that is not fully aligned to the reference. In some embodiments, the plurality of training sequencing reads is filtered to remove at least one training sequencing read that does not comprise a largest segment that is fully aligned to the reference. In some embodiments, the plurality of training sequencing reads is filtered to remove at least one training sequencing read that has a quality score that fails to meet a pre-determined criterion. In some embodiments, the plurality of training sequencing reads is filtered to remove at least one training sequencing read that has a length that differs from a reference length. In some embodiments, the reference length is a most common length of the plurality of training sequencing reads. In some embodiments, the plurality of training sequencing reads is filtered to remove at least one training sequencing read that comprises a pre-determined adapter sequence. In some embodiments, at least one of the plurality of training sequencing reads is padded with filler values such that the plurality of training sequencing reads has a substantially identical length. In some embodiments, the filler values are masking values comprising negative numbers. In some embodiments, the negative numbers are indicative of a class of trimmed flows. In some embodiments, the class of trimmed flows is selected from the group consisting of low quality flows, flows comprising three consecutive zero-signals, flows with errors, and flows with variants. In some embodiments, the method further comprises determining a likelihood of the sequence of the nucleic acid determined in (c) being correct. In some embodiments, the method further comprises determining a maximum likelihood h-mer length of the sequence of the nucleic acid.

In another aspect, the present disclosure provides a method for generating a trained algorithm, comprising: (a) obtaining a training set comprising a plurality of training sequencing signals and a plurality of training sequencing reads associated therewith, wherein training sequencing reads in the plurality of training sequencing reads are aligned to a reference genome, wherein the aligning is performed in flow space; and (b) using the training set to generate the trained algorithm, wherein the trained algorithm comprises a mapping between input sequencing signals and output sequencing reads comprising base calls.

In some embodiments, the plurality of training sequencing signals correspond to deoxyribonucleic acids (DNA) or ribonucleic acids (RNA). In some embodiments, the plurality of training sequencing signals are generated at least in part by performing flow sequencing of the DNA or RNA. In some embodiments, the plurality of sequencing signals comprise analog values produced by imaging a substrate. In some embodiments, the analog values comprise fluorescence signals. In some embodiments, the fluorescence signals correspond to discrete DNA extensions sensed from introduction of single nucleotide solutions in the flow sequencing. In some embodiments, the introduction of single nucleotide solutions in the flow sequencing is cyclic. In some embodiments, the introduction of single nucleotide solutions in the flow sequencing is acyclic.

In some embodiments, the trained algorithm comprises a trained machine learning algorithm. In some embodiments, the trained machine learning algorithm comprises a neural network, a support vector machine, a random forest, or a deep learning algorithm. In some embodiments, the trained machine learning algorithm comprises the neural network. In some embodiments, the neural network comprises a convolutional neural network.

In some embodiments, the aligning comprises using a set of common base calling variants. In some embodiments, the aligning comprises detecting contamination from a different genome. In some embodiments, the aligning comprises using indicators of pre-determined adapter sequences. In some embodiments, the plurality of training sequencing reads is filtered to remove at least one training sequencing read that is not fully aligned to the reference. In some embodiments, the plurality of training sequencing reads is filtered to remove at least one training sequencing read that does not comprise a largest segment that is fully aligned to the reference. In some embodiments, the plurality of training sequencing reads is filtered to remove at least one training sequencing read that has a quality score that fails to meet a pre-determined criterion. In some embodiments, the plurality of training sequencing reads is filtered to remove at least one training sequencing read that has a length that differs from a reference length. In some embodiments, the reference length is a most common length of the plurality of training sequencing reads. In some embodiments, the plurality of training sequencing reads is filtered to remove at least one training sequencing read that comprises a pre-determined adapter sequence. In some embodiments, at least one of the plurality of training sequencing reads is padded with filler values such that the plurality of training sequencing reads has a substantially identical length. In some embodiments, the filler values are masking values comprising negative numbers. In some embodiments, the negative numbers are indicative of a class of trimmed flows. In some embodiments, the class of trimmed flows is selected from the group consisting of low quality flows, flows comprising three consecutive zero-signals, flows with errors, and flows with variants.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein).

FIG. 1 shows an example of a method 100 for training a neural network to perform a first mapping between actual fragment sequencing signals of Escherichia coli and trusted fragment sequencing signals of E. coli, in accordance with some embodiments.

FIG. 2 shows an example of a method 200 for using a neural network (trained to apply the first mapping) for generating a second training set that may be used to map actual fragment sequencing signals of a certain person to trusted fragment sequencing signals of a reference human genome, in accordance with some embodiments.

FIG. 3 shows an example of a method 300 for estimating a genome of a subject, in accordance with some embodiments.

FIG. 4 shows an example of a method for hash-based alignment (e.g., according to operation 322), in accordance with some embodiments.

FIG. 5 shows an example of a neural network 500 that may be trained during method 100 and/or method 200—and that may be used during method 300, in accordance with some embodiments.

FIG. 6 shows an example of a method 600 for generating a training set, in accordance with some embodiments.

FIG. 7 shows an example of a method 700 for estimating a genome of a subject of a second genus, in accordance with some embodiments. The estimation is based on a first genus and method 700 may be referred to as a method for first genus-based estimation of a genome of a second genus.

FIG. 8 shows an example of a U-Net type neural network that is trained to estimate a genome of a subject of a second genus, in accordance with some embodiments.

FIG. 9 shows a computer system that is programmed or otherwise configured to implement methods provided herein, in accordance with some embodiments.

FIG. 10 shows an example of a graph 1000 that illustrates input signals 1001 and output signals 1002 of a neural network trained to estimate a genome of a subject of a second genus, in accordance with some embodiments.

FIG. 11 shows an example of an input signal histogram 1010 and an output signal histogram 1020 of a neural network trained to estimate a genome of a subject of a second genus, in accordance with some embodiments.

FIG. 12 shows an example of a method for estimating a genome of a subject of a genus, in accordance with some embodiments.

FIG. 13 shows an example of a method for estimating genomes of a plurality of subjects of a genus, in accordance with some embodiments.

FIG. 14 shows an example of a method for estimating a genome of a subject of a genus, in accordance with some embodiments.

FIG. 15 shows an example of a method for estimating a genome of a subject of a genus, in accordance with some embodiments.

FIG. 16 shows two examples of substrates (e.g., wafers) and segments thereof—wafer 1610 with segments thereof (e.g., arranged in a grid-like pattern), and wafer 1620 with segments thereof (e.g., arranged in a concentric circle pattern), in accordance with some embodiments.

FIG. 17 shows an example of a histogram plotted of the number of bases of each of the raw sequencing signals having a given amplitude (left panel) and a histogram of the processed signals showing narrow distributions of a number of bases of the processed sequences having amplitudes of about 0, 1, 2, and 3 (right panel), in accordance with some embodiments.

FIG. 18A shows the matrix output providing the probability that a given number of bases (“hmer”) were added during the specified nucleotide flow, in accordance with some embodiments. The cells highlighted in yellow indicate the most probable hmer value.

FIG. 18B shows a second matrix providing the probability that a given number of bases (“hmer”) were added during the specified nucleotide flow, in accordance with some embodiments. The most likely paths, representing the most likely haplotypes, are indicated by lines.

FIG. 19 shows a comparison of the precision and recall of each method for different types of sequences, in accordance with some embodiments. The methods were compared for HMER insertion/deletions (“indel”) of various lengths (top three plots), non-hmer indels (bottom left), and single nucleotide polymorphisms (bottom right, “SNP”).

FIG. 20 shows a processing pipeline for a variant calling method of the present disclosure, in accordance with some embodiments.

FIG. 21 shows the relation between predicted probability and read correct call rate for 2mer data, in accordance with some embodiments.

FIG. 22 illustrates an exemplary flow sequencing method that can be used to generate sequencing data, in accordance with some embodiments.

FIG. 23A illustrates an exemplary summary of detected signals after a number of exemplary flow cycles are performed, in accordance with some embodiments.

FIG. 23B illustrates an exemplary process for determining a preliminary sequence.

FIG. 24 illustrates an exemplary method for increasing sequencing read quality, in accordance with some embodiments.

FIG. 25A illustrates an exemplary plurality of sequencing reads, in accordance with some embodiments.

FIG. 25B illustrates a filtered set of sequencing reads, in accordance with some embodiments.

FIG. 25C illustrates a filtered and trimmed set of sequencing reads, in accordance with some embodiments.

FIG. 26A illustrates that three consecutive sequencing flow steps cannot all yield a signal of 0 indicating an absence of an incorporated nucleotide, in accordance with some embodiments.

FIG. 26B illustrates that three consecutive sequencing flow steps cannot all yield a signal of 0 indicating an absence of an incorporated nucleotide, in accordance with some embodiments.

FIG. 26C illustrates that three consecutive sequencing flow steps cannot all yield a signal of 0 indicating an absence of an incorporated nucleotide, in accordance with some embodiments.

FIG. 27 illustrates the read quality metrics for an exemplary sequencing read, in accordance with some embodiments.

FIG. 28A illustrates that quality issues may occur to an increasing percentage of reads as the number of flow steps increase, in accordance with some embodiments.

FIG. 28B illustrates a plurality of exemplary sequencing reads, in accordance with some embodiments.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

As used in the specification and claims, the singular form “a”, “an”, and “the” include plural references unless the context clearly dictates otherwise.

The term “at least partially” as used herein, generally refers to any fraction of a whole amount. For example, “at least partially” may refer to at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99.9%, or more of a whole amount.

The term “sequencing,” as used herein, generally refers to a process for generating or identifying a sequence of a biological molecule, such as a nucleic acid molecule or a polypeptide. Such sequence may be a nucleic acid sequence, which may include a sequence of nucleic acid bases (e.g., nucleobases). Sequencing methods may be massively parallel array sequencing (e.g., Illumina sequencing), which may be performed using template nucleic acid molecules immobilized on a support, such as a flow cell or beads. Sequencing methods may include, but are not limited to: high-throughput sequencing, next-generation sequencing, sequencing-by-synthesis, flow sequencing, massively-parallel sequencing, shotgun sequencing, single-molecule sequencing, nanopore sequencing, pyrosequencing, semiconductor sequencing, sequencing-by-ligation, sequencing-by-hybridization, RNA-Seq (Illumina), Digital Gene Expression (Helicos), Single Molecule Sequencing by Synthesis (SMSS) (Helicos), Clonal Single Molecule Array (Solexa), and Maxim-Gilbert sequencing.

The term “flow sequencing,” as used herein, generally refers to a sequencing-by-synthesis (SBS) process in which cyclic or acyclic introduction of single nucleotide solutions produce discrete DNA extensions that are sensed (e.g., by a detector that detects fluorescence signals from the DNA extensions).

The term “read,” as used herein, generally refers to a nucleic acid sequence, such as a sequencing read. A sequencing read may be an inferred sequence of nucleic acid bases (e.g., nucleotides) or base pairs obtained via a nucleic acid sequencing assay. A sequencing read may be generated by a nucleic acid sequencer, such as a massively parallel array sequencer (e.g., Illumina or Pacific Biosciences of California). A sequencing read may correspond to a portion, or in some cases all, of a genome of a subject. A sequencing read may be part of a collection of sequencing reads, which may be combined through, for example, alignment (e.g., to a reference genome), to yield a sequence of a genome of a subject.

The term “subject,” as used herein, generally refers to an individual or entity from which a biological sample (e.g., a biological sample that is undergoing or will undergo processing or analysis) may be derived. A subject may be an animal (e.g., mammal or non-mammal) or plant. The subject may be a human, dog, cat, horse, pig, bird, non-human primate, simian, farm animal, companion animal, sport animal, or rodent. A subject may be a patient. The subject may have or be suspected of having a disease or disorder, such as cancer (e.g., breast cancer, colorectal cancer, brain cancer, leukemia, lung cancer, skin cancer, liver cancer, pancreatic cancer, lymphoma, esophageal cancer or cervical cancer) or an infectious disease. Alternatively or in addition, a subject may be known to have previously have a disease or disorder. The subject may have or be suspected of having a genetic disorder such as achondroplasia, alpha-1 antitrypsin deficiency, antiphospholipid syndrome, autism, autosomal dominant polycystic kidney disease, Charcot-Marie-tooth, cri du chat, Crohn's disease, cystic fibrosis, Dercum disease, down syndrome, Duane syndrome, Duchenne muscular dystrophy, factor V Leiden thrombophilia, familial hypercholesterolemia, familial Mediterranean fever, fragile x syndrome, Gaucher disease, hemochromatosis, hemophilia, holoprosencephaly, Huntington's disease, Klinefelter syndrome, Marfan syndrome, myotonic dystrophy, neurofibromatosis, Noonan syndrome, osteogenesis imperfecta, Parkinson's disease, phenylketonuria, Poland anomaly, porphyria, progeria, retinitis pigmentosa, severe combined immunodeficiency, sickle cell disease, spinal muscular atrophy, Tay-Sachs, thalassemia, trimethylaminuria, Turner syndrome, velocardiofacial syndrome, WAGR syndrome, or Wilson disease. A subject may be undergoing treatment for a disease or disorder. A subject may be symptomatic or asymptomatic of a given disease or disorder. A subject may be healthy (e.g., not suspected of having disease or disorder). A subject may have one or more risk factors for a given disease. A subject may have a given weight, height, body mass index, or other physical characteristic. A subject may have a given ethnic or racial heritage, place of birth or residence, nationality, disease or remission state, family medical history, or other characteristic.

The term “sample,” as used herein, generally refers to a biological sample. As used herein, the term “biological sample” generally refers to a sample obtained from a subject. The biological sample may be obtained directly or indirectly from the subject. A sample may be obtained from a subject via any suitable method, including, but not limited to, spitting, swabbing, blood draw, biopsy, obtaining excretions (e.g., urine, stool, sputum, vomit, or saliva), excision, scraping, and puncture. A sample may be obtained from a subject by, for example, intravenously or intraarterially accessing the circulatory system, collecting a secreted biological sample (e.g., stool, urine, saliva, sputum, etc.), breathing, or surgically extracting a tissue (e.g., biopsy). The sample may be obtained by non-invasive methods including but not limited to: scraping of the skin or cervix, swabbing of the cheek, or collection of saliva, urine, feces, menses, tears, or semen. Alternatively, the sample may be obtained by an invasive procedure such as biopsy, needle aspiration, or phlebotomy. A sample may comprise a bodily fluid such as, but not limited to, blood (e.g., whole blood, red blood cells, leukocytes or white blood cells, platelets), plasma, serum, sweat, tears, saliva, sputum, urine, semen, mucus, synovial fluid, breast milk, colostrum, amniotic fluid, bile, bone marrow, interstitial or extracellular fluid, or cerebrospinal fluid. For example, a sample may be obtained by a puncture method to obtain a bodily fluid comprising blood and/or plasma. Such a sample may comprise both cells and cell-free nucleic acid material. Alternatively, the sample may be obtained from any other source including but not limited to blood, sweat, hair follicle, buccal tissue, tears, menses, feces, or saliva. The biological sample may be a tissue sample, such as a tumor biopsy. The sample may be obtained from any of the tissues provided herein including, but not limited to, skin, heart, lung, kidney, breast, pancreas, liver, intestine, brain, prostate, esophagus, muscle, smooth muscle, bladder, gall bladder, colon, or thyroid. The methods of obtaining provided herein include methods of biopsy including fine needle aspiration, core needle biopsy, vacuum assisted biopsy, large core biopsy, incisional biopsy, excisional biopsy, punch biopsy, shave biopsy or skin biopsy. The biological sample may comprise one or more cells. A biological sample may comprise one or more nucleic acid molecules such as one or more deoxyribonucleic acid (DNA) and/or ribonucleic acid (RNA) molecules (e.g., included within cells or not included within cells). Nucleic acid molecules may be included within cells. Alternatively or in addition, nucleic acid molecules may not be included within cells (e.g., cell-free nucleic acid molecules). The biological sample may be a cell-free sample.

The term “cell-free sample,” as used herein, generally refers to a sample that is substantially free of cells (e.g., less than 10% cells on a volume basis). A cell-free sample may be derived from any source (e.g., as described herein). For example, a cell-free sample may be derived from blood, sweat, urine, or saliva. For example, a cell-free sample may be derived from a tissue or bodily fluid. A cell-free sample may be derived from a plurality of tissues or bodily fluids. For example, a sample from a first tissue or fluid may be combined with a sample from a second tissue or fluid (e.g., while the samples are obtained or after the samples are obtained). In an example, a first fluid and a second fluid may be collected from a subject (e.g., at the same or different times) and the first and second fluids may be combined to provide a sample. A cell-free sample may comprise one or more nucleic acid molecules such as one or more DNA or RNA molecules.

A sample that is not a cell-free sample (e.g., a sample comprising one or more cells) may be processed to provide a cell-free sample. For example, a sample that includes one or more cells as well as one or more nucleic acid molecules (e.g., DNA and/or RNA molecules) not included within cells (e.g., cell-free nucleic acid molecules) may be obtained from a subject. The sample may be subjected to processing (e.g., as described herein) to separate cells and other materials from the nucleic acid molecules not included within cells, thereby providing a cell-free sample (e.g., comprising nucleic acid molecules not included within cells). The cell-free sample may then be subjected to further analysis and processing (e.g., as provided herein). Nucleic acid molecules not included within cells (e.g., cell-free nucleic acid molecules) may be derived from cells and tissues. For example, cell-free nucleic acid molecules may derive from a tumor tissue or a degraded cell (e.g., of a tissue of a body). Cell-free nucleic acid molecules may comprise any type of nucleic acid molecules (e.g., as described herein). Cell-free nucleic acid molecules may be double-stranded, single-stranded, or a combination thereof. Cell-free nucleic acid molecules may be released into a bodily fluid through secretion or cell death processes, e.g., cellular necrosis, apoptosis, or the like. Cell-free nucleic acid molecules may be released into bodily fluids from cancer cells (e.g., circulating tumor DNA (ctDNA)). Cell free nucleic acid molecules may also be fetal DNA circulating freely in a maternal blood stream (e.g., cell-free fetal nucleic acid molecules such as cffDNA). Alternatively or in addition to, cell-free nucleic acid molecules may be released into bodily fluids from healthy cells.

A biological sample may be obtained directly from a subject and analyzed without any intervening processing, such as, for example, sample purification or extraction. For example, a blood sample may be obtained directly from a subject by accessing the subject's circulatory system, removing the blood from the subject (e.g., via a needle), and transferring the removed blood into a receptacle. The receptacle may comprise reagents (e.g., anti-coagulants) such that the blood sample is useful for further analysis. Such reagents may be used to process the sample or analytes derived from the sample in the receptacle or another receptacle prior to analysis. In another example, a swab may be used to access epithelial cells on an oropharyngeal surface of the subject. Following obtaining the biological sample from the subject, the swab containing the biological sample may be contacted with a fluid (e.g., a buffer) to collect the biological fluid from the swab.

Any suitable biological sample that comprises one or more nucleic acid molecules may be obtained from a subject. A sample (e.g., a biological sample or cell-free biological sample) suitable for use according to the methods provided herein may be any material comprising tissues, cells, degraded cells, nucleic acids, genes, gene fragments, expression products, gene expression products, and/or gene expression product fragments of an individual to be tested. A biological sample may be solid matter (e.g., biological tissue) or may be a fluid (e.g., a biological fluid). In general, a biological fluid may include any fluid associated with living organisms. Non-limiting examples of a biological sample include blood (or components of blood—e.g., white blood cells, red blood cells, platelets) obtained from any anatomical location (e.g., tissue, circulatory system, bone marrow) of a subject, cells obtained from any anatomical location of a subject, skin, heart, lung, kidney, breath, bone marrow, stool, semen, vaginal fluid, interstitial fluids derived from tumorous tissue, breast, pancreas, cerebral spinal fluid, tissue, throat swab, biopsy, placental fluid, amniotic fluid, liver, muscle, smooth muscle, bladder, gall bladder, colon, intestine, brain, cavity fluids, sputum, pus, microbiota, meconium, breast milk, prostate, esophagus, thyroid, serum, saliva, urine, gastric and digestive fluid, tears, ocular fluids, sweat, mucus, earwax, oil, glandular secretions, spinal fluid, hair, fingernails, skin cells, plasma, nasal swab or nasopharyngeal wash, spinal fluid, cord blood, emphatic fluids, and/or other excretions or body tissues. Methods for determining sample suitability and/or adequacy are provided. A sample may include, but is not limited to, blood, plasma, tissue, cells, degraded cells, cell-free nucleic acid molecules, and/or biological material from cells or derived from cells of an individual such as cell-free nucleic acid molecules. The sample may be a heterogeneous or homogeneous population of cells, tissues, or cell-free biological material. The biological sample may be obtained using any method that can provide a sample suitable for the analytical methods described herein.

A sample (e.g., a biological sample or cell-free biological sample) may undergo one or more processes in preparation for analysis, including, but not limited to, filtration, centrifugation, selective precipitation, permeabilization, isolation, agitation, heating, purification, and/or other processes. For example, a sample may be filtered to remove contaminants or other materials. In an example, a sample comprising cells may be processed to separate the cells from other material in the sample. Such a process may be used to prepare a sample comprising only cell-free nucleic acid molecules. Such a process may consist of a multi-step centrifugation process. Multiple samples, such as multiple samples from the same subject (e.g., obtained in the same or different manners from the same or different bodily locations, and/or obtained at the same or different times (e.g., seconds, minutes, hours, days, weeks, months, or years apart)) or multiple samples from different subjects may be obtained for analysis as described herein. In an example, the first sample is obtained from a subject before the subject undergoes a treatment regimen or procedure and the second sample is obtained from the subject after the subject undergoes the treatment regimen or procedure. Alternatively or in addition to, multiple samples may be obtained from the same subject at the same or approximately the same time. Different samples obtained from the same subject may be obtained in the same or different manner. For example, a first sample may be obtained via a biopsy and a second sample may be obtained via a blood draw. Samples obtained in different manners may be obtained by different medical professionals, using different techniques, at different times, and/or at different locations. Different samples obtained from the same subject may be obtained from different areas of a body. For example, a first sample may be obtained from a first area of a body (e.g., a first tissue) and a second sample may be obtained from a second area of the body (e.g., a second tissue).

A biological sample as used herein (e.g., a biological sample comprising one or more nucleic acid molecules) may not be purified when provided in a reaction vessel. Furthermore, for a biological sample comprising one or more nucleic acid molecules, the one or more nucleic acid molecules may not be extracted when the biological sample is provided to a reaction vessel. For example, ribonucleic acid (RNA) and/or deoxyribonucleic acid (DNA) molecules of a biological sample may not be extracted from the biological sample when providing the biological sample to a reaction vessel. Moreover, a target nucleic acid (e.g., a target RNA or target DNA molecules) present in a biological sample may not be concentrated when providing the biological sample to a reaction vessel. Alternatively, a biological sample may be purified and/or nucleic acid molecules may be isolated from other materials in the biological sample.

The term “nucleic acid,” or “polynucleotide,” as used herein, generally refers to a molecule comprising one or more nucleic acid subunits, or nucleotides. A nucleic acid may include one or more nucleotides selected from adenosine (A), cytosine (C), guanine (G), thymine (T) and uracil (U), or variants thereof. A nucleotide generally includes a nucleoside and at least 1, 2, 3, 4, 5, 6, 7, 8, 9, 10, or more phosphate (PO₃) groups. A nucleotide can include a nucleobase, a five-carbon sugar (either ribose or deoxyribose), and one or more phosphate groups.

Ribonucleotides are nucleotides in which the sugar is ribose. Deoxyribonucleotides are nucleotides in which the sugar is deoxyribose. A nucleotide can be a nucleoside monophosphate or a nucleoside polyphosphate. A nucleotide can be a deoxyribonucleoside polyphosphate, such as, e.g., a deoxyribonucleoside triphosphate (dNTP), which can be selected from deoxyadenosine triphosphate (dATP), deoxycytidine triphosphate (dCTP), deoxyguanosine triphosphate (dGTP), uridine triphosphate (dUTP) and deoxythymidine triphosphate (dTTP) dNTPs, that include detectable tags, such as luminescent tags or markers (e.g., fluorophores). A nucleotide can include any subunit that can be incorporated into a growing nucleic acid strand. Such subunit can be an A, C, G, T, or U, or any other subunit that is specific to one or more complementary A, C, G, T or U, or complementary to a purine (i.e., A or G, or variant thereof) or a pyrimidine (i.e., C, T or U, or variant thereof). In some examples, a nucleic acid is deoxyribonucleic acid (DNA), ribonucleic acid (RNA), or derivatives or variants thereof. A nucleic acid may be single-stranded or double-stranded. In some cases, a nucleic acid molecule is circular.

The terms “nucleic acid molecule,” “nucleic acid sequence,” “nucleic acid fragment,” “oligonucleotide” and “polynucleotide,” as used herein, generally refer to a polynucleotide that may have various lengths, such as either deoxyribonucleotides or ribonucleotides (RNA), or analogs thereof. Nucleic acids may have any three-dimensional structure, and may perform any function, known or unknown. A nucleic acid molecule can have a length of at least about 10 bases, 20 bases, 30 bases, 40 bases, 50 bases, 100 bases, 200 bases, 300 bases, 400 bases, 500 bases, 1 kilobase (kb), 2 kb, 3, kb, 4 kb, 5 kb, 10 kb, 50 kb, or more. An oligonucleotide is typically composed of a specific sequence of four nucleotide bases: adenine (A); cytosine (C); guanine (G); and thymine (T) (uracil (U) for thymine (T) when the polynucleotide is RNA). Thus, the term “oligonucleotide sequence” is the alphabetical representation of a polynucleotide molecule; alternatively, the term may be applied to the polynucleotide molecule itself. This alphabetical representation can be input into databases in a computer having a central processing unit and used for bio informatics applications such as functional genomics and homology searching.

Oligonucleotides may include one or more nonstandard nucleotide(s), nucleotide analog(s), and/or modified nucleotides. Non-limiting examples of nucleic acids include DNA, RNA, genomic DNA (e.g., gDNA such as sheared gDNA), cell-free DNA (e.g., cfDNA), synthetic DNA/RNA, coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, complementary DNA (cDNA), recombinant nucleic acids, branched nucleic acids, plasmids, vectors, isolated DNA of any sequence, isolated RNA of any sequence, nucleic acid probes, and primers. A nucleic acid may comprise one or more modified nucleotides, such as methylated nucleotides and nucleotide analogs. If present, modifications to the nucleotide structure may be made before or following assembly of the nucleic acid. The sequence of nucleotides of a nucleic acid may be interrupted by non-nucleotide components. A nucleic acid may be further modified following polymerization, such as by conjugation or binding with a reporter agent.

A target nucleic acid or sample nucleic acid as described herein may be amplified to generate an amplified product. A target nucleic acid may be a target RNA or a target DNA. When the target nucleic acid is a target RNA, the target RNA may be any type of RNA, including types of RNA described elsewhere herein. The target RNA may be viral RNA and/or tumor RNA. A viral RNA may be pathogenic to a subject. Non-limiting examples of pathogenic viral RNA include human immunodeficiency virus I (HIV I), human immunodeficiency virus n (HIV 11), orthomyxoviruses, Ebola virus. Dengue virus, influenza viruses (e.g., H1N1, H3N2, H7N9, or H5N1), herpes virus, hepatitis A virus, hepatitis B virus, hepatitis C (e.g., armored RNA-HCV virus) virus, hepatitis D virus, hepatitis E virus, hepatitis G virus, Epstein-Barr virus, mononucleosis virus, cytomegalovirus, SARS virus, West Nile Fever virus, polio virus, and measles virus.

A biological sample may comprise a plurality of target nucleic acid molecules. For example, a biological sample may comprise a plurality of target nucleic acid molecules from a single subject. In another example, a biological sample may comprise a first target nucleic acid molecule from a first subject and a second target nucleic acid molecule from a second subject.

As used herein, a “double-stranded” molecule is a molecule comprising a region of double-stranded nucleic acid molecule. In some embodiments, double-stranded is 100% double-stranded. In some embodiments, double-stranded is at least 50, 55, 60, 65, 70, 75, 80, 85, 90, 92, 95, 97, 99 or 100% double stranded. Each possibility represents a separate embodiment of the invention. In some embodiments, a double-stranded molecule comprises a stretch of double-stranded nucleotides that is at least 1, 2, 3, 4, 5, 6, 7, 8, 9, 10, 12, 14, 15, 16, 18, 20, 25, 30, 35, 40, 45 or 50 bases long. Each possibility represents a separate embodiment of the invention. In some embodiments, the double-stranded molecule comprises a single-stranded overhang. In some embodiments, the overhang is not more than 1, 2, 3, 4, 5, 6, 7, 8, 9 or 10 bases is length. Each possibility represents a separate embodiment of the invention.

The term “nucleotide,” as used herein, generally refers to a substance including a base (e.g., a nucleobase), sugar moiety, and phosphate moiety. A nucleotide may comprise a free base with attached phosphate groups. A substance including a base with three attached phosphate groups may be referred to as a nucleoside triphosphate. When a nucleotide is being added to a growing nucleic acid molecule strand, the formation of a phosphodiester bond between the proximal phosphate of the nucleotide to the growing chain may be accompanied by hydrolysis of a high-energy phosphate bond with release of the two distal phosphates as a pyrophosphate. The nucleotide may be naturally occurring or non-naturally occurring (e.g., a modified or engineered nucleotide).

The term “nucleotide analogs,” as used herein, may include, but are not limited to, diaminopurine, 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, 2,6-diaminopurine, phosphoroselenoate nucleic acids, and the like. In some cases, nucleotides may include modifications in their phosphate moieties, including modifications to a triphosphate moiety. Additional, non-limiting examples of modifications include phosphate chains of greater length (e.g., a phosphate chain having 4, 5, 6, 7, 8, 9, 10, or more than 10 phosphate moieties), modifications with thiol moieties (e.g., alpha-thio triphosphate and beta-thiotriphosphates) or modifications with selenium moieties (e.g., phosphoroselenoate nucleic acids). Nucleic acid molecules may also be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone. Nucleic acid molecules may also contain amine-modified groups, such as aminoallyl-dUTP (aa-dUTP) and aminohexhylacrylamide-dCTP (aha-dCTP) to allow covalent attachment of amine reactive moieties, such as N-hydroxysuccinimide esters (NHS). Alternatives to standard DNA base pairs or RNA base pairs in the oligonucleotides of the present disclosure can provide higher density in bits per cubic millimeter (mm), higher safety (e.g., resistance to accidental or purposeful synthesis of natural toxins), easier discrimination in photo-programmed polymerases, or lower secondary structure. Nucleotide analogs may be capable of reacting or bonding with detectable moieties for nucleotide detection. An analog to a cleavable base may be the non-cleavable alternative to the base. For example, thymine is a non-cleavable analog to uracil and adenine is a non-cleavable analog of inosine.

The term “free nucleotide analog” as used herein, generally refers to a nucleotide analog that is not coupled to an additional nucleotide or nucleotide analog. Free nucleotide analogs may be incorporated into the growing nucleic acid chain by primer extension reactions.

As used herein, the term “primer” or “primer molecule” generally refers to a polynucleotide which is complementary to a portion of a template nucleic acid molecule. For example, a primer may be complementary to a portion of a strand of a template nucleic acid molecule. The primer may be a strand of nucleic acid that serves as a starting point for nucleic acid synthesis, such as a primer extension reaction which may be a component of a nucleic acid reaction (e.g., nucleic acid amplification reaction such as PCR). A primer may hybridize to a template strand and nucleotides (e.g., canonical nucleotides or nucleotide analogs) may then be added to the end(s) of a primer, sometimes with the aid of a polymerizing enzyme such as a polymerase. Thus, during replication of a DNA sample, an enzyme that catalyzes replication may start replication at the 3′-end of a primer attached to the DNA sample and copy the opposite strand. A primer (e.g., oligonucleotide) may have one or more functional groups that may be used to couple the primer to a support or carrier, such as a bead or particle. The length of the primer may be between 8 nucleotide bases to 50 nucleotide bases. The length of the primer may be greater than or equal to 6 nucleotide bases, 7 nucleotide bases, 8 nucleotide bases, 9 nucleotide bases, 10 nucleotide bases, 11 nucleotide bases, 12 nucleotide bases, 13 nucleotide bases, 14 nucleotide bases, 15 nucleotide bases, 16 nucleotide bases, 17 nucleotide bases, 18 nucleotide bases, 19 nucleotide bases, 20 nucleotide bases, 21 nucleotide bases, 22 nucleotide bases, 23 nucleotide bases, 24 nucleotide bases, 25 nucleotide bases, 26 nucleotide bases, 27 nucleotide bases, 28 nucleotide bases, 29 nucleotide bases, 30 nucleotide bases, 31 nucleotide bases, 32 nucleotide bases, 33 nucleotide bases, 34 nucleotide bases, 35 nucleotide bases, 37 nucleotide bases, 40 nucleotide bases, 42 nucleotide bases, 45 nucleotide bases, 47 nucleotide bases, or 50 nucleotide bases.

A primer may be completely or partially complementary to a template nucleic acid. A primer may exhibit sequence identity or homology or complementarity to the template nucleic acid. The homology or sequence identity or complementarity between the primer and a template nucleic acid may be based on the length of the primer. For example, if the primer length is about 20 nucleic acids, it may contain 10 or more contiguous nucleic acid bases complementary to the template nucleic acid.

The term “% sequence identity” may be used interchangeably herein with the term “% identity” and may refer to the level of nucleotide sequence identity between two or more nucleotide sequences, when aligned using a sequence alignment program. As used herein, 80% identity may be the same thing as 80% sequence identity determined by a defined algorithm, and means that a given sequence is at least 80% identical to another length of another sequence. The % identity may be selected from, e.g., at least 60%, at least 65%, at least 70%, at least 75%, at least 80%, at least 85%, at least 90%, at least 95%, or at least 99% or more sequence identity to a given sequence. The % identity may be in the range of, e.g., about 60% to about 70%, about 70% to about 80%, about 80% to about 85%, about 85% to about 90%, about 90% to about 95%, or about 95% to about 99%.

The terms “% sequence homology” or “percent sequence homology” or “percent sequence identity” may be used interchangeably herein with the terms “% homology,” “% sequence identity,” or “% identity” and may refer to the level of nucleotide sequence homology between two or more nucleotide sequences, when aligned using a sequence alignment program. For example, as used herein, 80% homology may be the same thing as 80% sequence homology determined by a defined algorithm, and accordingly a homologue of a given sequence has greater than 80% sequence homology over a length of the given sequence. The % homology may be selected from, e.g., at least 60%, at least 65%, at least 70%, at least 75%, at least 80%, at least 85%, at least 90%, at least 95%, or at least 99% or more sequence homology to a given sequence. The % homology may be in the range of, e.g., about 60% to about 70%, about 70% to about 80%, about 80% to about 85%, about 85% to about 90%, about 90% to about 95%, or about 95% to about 99%.

The term “primer extension,” as used herein, generally refers to the binding of a primer to a strand of the template nucleic acid, followed by elongation of the primer(s). It may also include, denaturing of a double-stranded nucleic acid and the binding of a primer strand to either one or both of the denatured template nucleic acid strands, followed by elongation of the primer(s). Primer extension reactions may be used to incorporate nucleotides or nucleotide analogs to a primer in template-directed fashion by using enzymes (polymerizing enzymes).

The term “polymerizing enzyme” or “polymerase,” as used herein, generally refers to any enzyme capable of catalyzing a polymerization reaction. A polymerizing enzyme may be used to extend a nucleic acid primer paired with a template strand by incorporation of nucleotides or nucleotide analogs. A polymerizing enzyme may add a new strand of DNA by extending the 3′ end of an existing nucleotide chain, adding new nucleotides matched to the template strand one at a time via the creation of phosphodiester bonds. The polymerase used herein can have strand displacement activity or non-strand displacement activity. Examples of polymerases include, without limitation, a nucleic acid polymerase. The polymerase can be naturally occurring or synthesized. In some cases, a polymerase has relatively high processivity, namely the capability of the polymerase to consecutively incorporate nucleotides into a nucleic acid template without releasing the nucleic acid template. An example polymerase is a Φ29 polymerase or a derivative thereof. A polymerase can be a polymerization enzyme. In some cases, a transcriptase or a ligase is used (i.e., enzymes which catalyze the formation of a bond). Examples of polymerases include, but are not limited to, a DNA polymerase, an RNA polymerase, a thermostable polymerase, a wild-type polymerase, a modified polymerase, E. coli DNA polymerase I, T7 DNA polymerase, bacteriophage T4 DNA polymerase Φ29 (phi29) DNA polymerase, Taq polymerase, Tth polymerase, Tli polymerase, Pfu polymerase, Pwo polymerase, VENT polymerase, DEEPVENT polymerase, EX-Taq polymerase, LA-Taq polymerase, Sso polymerase, Poc polymerase, Pab polymerase, Mth polymerase, ES4 polymerase, Tru polymerase, Tac polymerase, Tne polymerase, Tma polymerase, Tea polymerase, Tih polymerase, Tfi polymerase, Platinum Taq polymerases, Tbr polymerase, Tfl polymerase, Pfutubo polymerase, Pyrobest polymerase, Pwo polymerase, KOD polymerase, Bst polymerase, Sac polymerase, Klenow fragment, polymerase with 3′ to 5′ exonuclease activity, and variants, modified products and derivatives thereof. In some cases, the polymerase is a single subunit polymerase. The polymerase can have high processivity, namely the capability of the polymerase to consecutively incorporate nucleotides into a nucleic acid template without releasing the nucleic acid template. In some cases, a polymerase is a polymerase modified to accept dideoxynucleotide triphosphates, such as for example, Taq polymerase having a 667Y mutation (see e.g., Tabor et al, PNAS, 1995, 92, 6339-6343, which is herein incorporated by reference in its entirety for all purposes). In some cases, a polymerase is a polymerase having a modified nucleotide binding, which may be useful for nucleic acid sequencing, with non-limiting examples that include ThermoSequenas polymerase (GE Life Sciences), AmpliTaq FS (ThermoFisher) polymerase and Sequencing Pol polymerase (Jena Bioscience). In some cases, the polymerase is genetically engineered to have discrimination against dideoxynucleotides, such, as for example, Sequenase DNA polymerase (ThermoFisher).

A polymerase may be Family A polymerase or a Family B DNA polymerase. Family A polymerases include, for example, Taq, Klenow, and Bst polymerases. Family B polymerases include, for example, Vent(exo-) and Therminator polymerases. Family B polymerases are known to accept more varied nucleotide substrates than Family A polymerases. Family A polymerases are used widely in sequencing by synthesis methods, likely due to their high processivity and fidelity.

The term “complementary sequence,” as used herein, generally refers to a sequence that hybridizes to another sequence. Hybridization between two single-stranded nucleic acid molecules may involve the formation of a double-stranded structure that is stable under certain conditions. Two single-stranded polynucleotides may be considered to be hybridized if they are bonded to each other by two or more sequentially adjacent base pairings. A substantial proportion of nucleotides in one strand of a double-stranded structure may undergo Watson-Crick base-pairing with a nucleoside on the other strand. Hybridization may also include the pairing of nucleoside analogs, such as deoxyinosine, nucleosides with 2-aminopurine bases, and the like, that may be employed to reduce the degeneracy of probes, whether or not such pairing involves formation of hydrogen bonds.

The term “support or “substrate,” as used herein, generally refers to a solid or semi-solid support on which reagents such as nucleic acid molecules may be immobilized, such as a slide, a bead, a resin, a chip, an array, a matrix, a membrane, a nanopore, or a gel. Nucleic acid molecules may be synthesized, attached, ligated, or otherwise immobilized. Nucleic acid molecules may be immobilized on a substrate by any method including, but not limited to, physical adsorption, by ionic or covalent bond formation, or combinations thereof. A substrate may be 2-dimensional (e.g., a planar 2D substrate) or 3-dimensional. In some cases, a substrate may be a component of a flow cell and/or may be included within or adapted to be received by a sequencing instrument. A substrate may include a polymer, a glass, or a metallic material. Examples of substrates include a membrane, a planar substrate, a microtiter plate, a bead (e.g., a magnetic bead), a filter, a test strip, a slide, a cover slip, and a test tube. A substrate may comprise organic polymers such as polystyrene, polyethylene, polypropylene, polyfluoroethylene, polyethyleneoxy, and polyacrylamide (e.g., polyacrylamide gel), as well as co-polymers and grafts thereof. A substrate may comprise latex or dextran. A substrate may also be inorganic, such as glass, silica, gold, controlled-pore-glass (CPG), or reverse-phase silica. The configuration of a support may be, for example, in the form of beads, spheres, particles, granules, a gel, a porous matrix, or a substrate. In some cases, a substrate may be a single solid or semi-solid article (e.g., a single particle), while in other cases a substrate may comprise a plurality of solid or semi-solid articles (e.g., a collection of particles). Substrates may be planar, substantially planar, or non-planar. Substrates may be porous or non-porous, and may have swelling or non-swelling characteristics. A substrate may be shaped to comprise one or more wells, depressions, or other containers, vessels, features, or locations. A plurality of substrates may be configured in an array at various locations. A substrate may be addressable (e.g., for robotic delivery of reagents), or by detection approaches, such as scanning by laser illumination and confocal or deflective light gathering. For example, a substrate may be in optical and/or physical communication with a detector. Alternatively, a substrate may be physically separated from a detector by a distance. An amplification substrate (e.g., a bead) can be placed within or on another substrate (e.g., within a well of a second support) The substrate may have surface properties, such as textures, patterns, microstructure coatings, surfactants, or any combination thereof to retain the amplification substrate (e.g., bead) at a desired location (such as in a position to be in operative communication with a detector). The detector of bead-based supports may be configured to maintain substantially the same read rate independent of the size of the bead. The support may be in optical communication with the detector, may be physically in contact with the detector, may be separated from the detector by a distance, or any combination thereof. The support may have a plurality of independently addressable locations. The nucleic acid molecules may be immobilized to the support at a given independently addressable location of the plurality of independently addressable locations. Immobilization of each of the plurality of nucleic acid molecules to the support may be aided by the use of an adaptor. The support may be optically coupled to the detector. Immobilization on the support may be aided by an adaptor.

The term “solid support” refers to any artificial solid structure, including any solid support or substrate. Examples of solid supports include, but are not limited to beads, resins, gels, hydrogels, colloids, particles or nanoparticles. For example, a solid support may be a bead. Alternatively, the solid support may be a surface. For example, a solid support may comprise a bead coupled to a surface. Alternatively, the solid support may be a resin. The solid support may be isolatable. The solid support may be tagged. The solid support may be magnetic and isolatable with a magnet. Alternatively or in addition, the solid support may be isolated by centrifugation or some other force that separates by weight, size or some other measurable quantity.

A support (e.g., a solid support) may be or comprise a particle. A particle may be a bead. A bead may comprise any suitable material such as glass or ceramic, one or more polymers, and/or metals. Examples of suitable polymers include, but are not limited to, nylon, polytetrafluoroethylene, polystyrene, polyacrylamide, agarose, cellulose, cellulose derivatives, or dextran. Examples of suitable metals include paramagnetic metals, such as iron. A bead may be magnetic or non-magnetic. For example, a bead may comprise one or more polymers bearing one or more magnetic labels. A magnetic bead may be manipulated (e.g., moved between locations or physically constrained to a given location, e.g., of a reaction vessel such as a flow cell chamber) using electromagnetic forces. A bead may have any useful shape, including, for example, a shape that is approximately cubic, spherical, ellipsoidal, dumbbell-shaped, or any other shape. For example, a bead may be approximately spherical in shape. A bead may have one or more different dimensions including a diameter. A dimension of the bead (e.g., a diameter of the bead) may be less than about 1 mm, less than about 0.1 mm, less than about 0.01 mm, less than about 0.005 mm, less than about 1 nm, less than about 1 μm, or smaller. A dimension of the bead (e.g., a diameter of the bead) may be between about 1 nm to about 100 nm, about 1 μm to about 100 μm, about 1 mm to about 100 mm. A collection of beads may comprise one or more beads having the same or different characteristics. For example, a first bead of a collection of beads may have a first diameter and a second bead of the collection of beads may have a second diameter. The first diameter may be the same or approximately the same as or different from the second diameter. Similarly, the first bead may have the same or a different shape and composition than a second bead.

The term “label,” as used herein, generally refers to a moiety that is capable of coupling with a species, such as, for example, a nucleotide analog. In some cases, a label may be a detectable label that emits a signal (or reduces an already emitted signal) that can be detected. In some cases, such a signal may be indicative of incorporation of one or more nucleotides or nucleotide analogs. In some cases, a label may be coupled to a nucleotide or nucleotide analog, which nucleotide or nucleotide analog may be used in a primer extension reaction. In some cases, the label may be coupled to a nucleotide analog after the primer extension reaction. The label, in some cases, may be reactive specifically with a nucleotide or nucleotide analog. Coupling may be covalent or non-covalent (e.g., via ionic interactions, Van der Waals forces, etc.). In some cases, coupling may be via a linker, which may be cleavable, such as photo-cleavable (e.g., cleavable under ultra-violet light), chemically-cleavable (e.g., via a reducing agent, such as dithiothreitol (DTT), tris(2-carboxyethyl)phosphine (TCEP)) or enzymatically cleavable (e.g., via an esterase, lipase, peptidase, or protease).

In some cases, the label may be optically active (e.g., luminescent, e.g., fluorescent or phosphorescent). In some embodiments, an optically-active label is an optically-active dye (e.g., fluorescent dye). Dyes and labels may be incorporated into nucleic acid sequences. Dyes and labels may also be incorporated into linkers, such as linkers for linking one or more beads to one another. For example, labels such as fluorescent moieties may be linked to nucleotides or nucleotide analogs via a linker. Non-limiting examples of dyes include SYBR green, SYBR blue, DAPI, propidium iodine, Hoechst, SYBR gold, ethidium bromide, acridine, proflavine, acridine orange, acriflavine, fluorocoumarin, ellipticine, daunomycin, chloroquine, distamycin D, chromomycin, homidium, mithramycin, ruthenium polypyridyls, anthramycin, phenanthridines and acridines, propidium iodide, hexidium iodide, dihydroethidium, ethidium homodimer-1 and -2, ethidium monoazide, ACMA, Hoechst 33258, Hoechst 33342, Hoechst 34580, DAPI, acridine orange, 7-AAD, actinomycin D, LDS751, hydroxystilbamidine, SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-1, TO-PRO-3, TO-PRO-5, JO-PRO-1, LO-PRO-1, YO-PRO-1, YO-PRO-3, PicoGreen, OliGreen, RiboGreen, SYBR Gold, SYBR Green I, SYBR Green II, SYBR DX, SYTO labels (e.g., SYTO-40, -41, -42, -43, -44, and -45 (blue); SYTO-13, -16, -24, -21, -23, -12, -11, -20, -22, -15, -14, and -25 (green); SYTO-81, -80, -82, -83, -84, and -85 (orange); and SYTO-64, -17, -59, -61, -62, -60, and -63 (red)), fluorescein, fluorescein isothiocyanate (FITC), tetramethyl rhodamine isothiocyanate (TRITC), rhodamine, tetramethyl rhodamine, R-phycoerythrin, Cy-2, Cy-3, Cy-3.5, Cy-5, Cy5.5, Cy-7, Texas Red, Phar-Red, allophycocyanin (APC), Sybr Green I, Sybr Green II, Sybr Gold, CellTracker Green, 7-AAD, ethidium homodimer I, ethidium homodimer II, ethidium homodimer III, ethidium bromide, umbelliferone, eosin, green fluorescent protein, erythrosin, coumarin, methyl coumarin, pyrene, malachite green, stilbene, lucifer yellow, cascade blue, dichlorotriazinylamine fluorescein, dansyl chloride, fluorescent lanthanide complexes such as those including europium and terbium, carboxy tetrachloro fluorescein, 5 and/or 6-carboxy fluorescein (FAM), VIC, 5- (or 6-) iodoacetamidofluorescein, 5-{[2(and 3)-5-(Acetylmercapto)-succinyl]amino} fluorescein (SAMSA-fluorescein), lissamine rhodamine B sulfonyl chloride, 5 and/or 6 carboxy rhodamine (ROX), 7-amino-methyl-coumarin, 7-Amino-4-methylcoumarin-3-acetic acid (AMCA), BODIPY fluorophores, 8-methoxypyrene-1,3,6-trisulfonic acid trisodium salt, 3,6-Disulfonate-4-amino-naphthalimide, phycobiliproteins, AlexaFluor labels (e.g., AlexaFluor 350, 405, 430, 488, 532, 546, 555, 568, 594, 610, 633, 635, 647, 660, 680, 700, 750, and 790 dyes), DyLight labels (e.g., DyLight 350, 405, 488, 550, 594, 633, 650, 680, 755, and 800 dyes), Black Hole Quencher Dyes (Biosearch Technologies) (e.g., BH1-0, BHQ-1, BHQ-3, and BHQ-10), QSY Dye fluorescent quenchers (Molecular Probes/Invitrogen) (e.g., QSY7, QSY9, QSY21, and QSY35), Dabcyl, Dabsyl, Cy5Q, Cy7Q, Dark Cyanine dyes (GE Healthcare), Dy-Quenchers (Dyomics) (e.g., DYQ-660 and DYQ-661), ATTO fluorescent quenchers (ATTO-TEC GmbH) (e.g., ATTO 540Q, ATTO 580Q, ATTO 612Q, Atto532 [e.g., Atto 532 succinimidyl ester], and Atto633), and other fluorophores and/or quenchers. A fluorescent dye may be excited by application of energy corresponding to the visible region of the electromagnetic spectrum (e.g., between about 430-770 nanometers (nm)). Excitation may be done using any useful apparatus, such as a laser and/or light emitting diode. Optical elements including, but not limited to, mirrors, waveplates, filters, monochromators, gratings, beam splitters, and lenses may be used to direct light to or from a fluorescent dye. A fluorescent dye may emit light (e.g., fluoresce) in the visible region of the electromagnetic spectrum ((e.g., between about 430-770 nm). A fluorescent dye may be excited over a single wavelength or a range of wavelengths. A fluorescent dye may be excitable by light in the red region of the visible portion of the electromagnetic spectrum (about 625-740 nm) (e.g., have an excitation maximum in the red region of the visible portion of the electromagnetic spectrum). Alternatively or in addition to, fluorescent dye may be excitable by light in the green region of the visible portion of the electromagnetic spectrum (about 500-565 nm) (e.g., have an excitation maximum in the green region of the visible portion of the electromagnetic spectrum). A fluorescent dye may emit signal in the red region of the visible portion of the electromagnetic spectrum (about 625-740 nm) (e.g., have an emission maximum in the red region of the visible portion of the electromagnetic spectrum). Alternatively or in addition to, fluorescent dye may emit signal in the green region of the visible portion of the electromagnetic spectrum (about 500-565 nm) (e.g., have an emission maximum in the green region of the visible portion of the electromagnetic spectrum).

In some examples, labels may be nucleic acid intercalator dyes. Examples include, but are not limited to ethidium bromide, YOYO-1, SYBR Green, and EvaGreen. The near-field interactions between energy donors and energy acceptors, between intercalators and energy donors, or between intercalators and energy acceptors can result in the generation of unique signals or a change in the signal amplitude. For example, such interactions can result in quenching (i.e., energy transfer from donor to acceptor that results in non-radiative energy decay) or Forster resonance energy transfer (FRET) (i.e., energy transfer from the donor to an acceptor that results in radiative energy decay). Other examples of labels include electrochemical labels, electrostatic labels, colorimetric labels and mass tags.

Labels may be quencher molecules. The term “quencher,” as used herein refers to a molecule that may be energy acceptors. A quencher may be a molecule that can reduce an emitted signal. For example, a template nucleic acid molecule may be designed to emit a detectable signal. Incorporation of a nucleotide or nucleotide analog comprising a quencher can reduce or eliminate the signal, which reduction or elimination is then detected. Luminescence from labels (e.g., fluorescent moieties, such as fluorescent moieties linked to nucleotides or nucleotide analogs) may also be quenched (e.g., by incorporation of other nucleotides that may or may not comprise labels). In some cases, as described elsewhere herein, labeling with a quencher can occur after nucleotide or nucleotide analog incorporation. In some cases, the label may be a type that does not self-quench or exhibit proximity quenching. Non-limiting examples of a label type that does not self-quench or exhibit proximity quenching include Bimane derivatives such as Monobromobimane. The term “proximity quenching,” as used herein, generally refers to a phenomenon where one or more dyes near each other may exhibit lower fluorescence as compared to the fluorescence they exhibit individually. In some cases, the dye may be subject to proximity quenching wherein the donor dye and acceptor dye are within 1 nm to 50 nm of each other. Examples of quenchers include, but are not limited to, Black Hole Quencher Dyes (Biosearch Technologies) (e.g., BH1-0, BHQ-1, BHQ-3, and BHQ-10), QSY Dye fluorescent quenchers (Molecular Probes/Invitrogen) (e.g., QSY7, QSY9, QSY21, and QSY35), Dabcyl, Dabsyl, Cy5Q, Cy7Q, Dark Cyanine dyes (GE Healthcare), Dy-Quenchers (Dyomics) (e.g., DYQ-660 and DYQ-661), and ATTO fluorescent quenchers (ATTO-TEC GmbH) (e.g., ATTO 540Q, ATTO 580Q, and ATTO 612Q). Fluorophore donor molecules may be used in conjunction with a quencher. Examples of fluorophore donor molecules that can be used in conjunction with quenchers include, but are not limited to, fluorophores such as Cy3B, Cy3, or Cy5; Dy-Quenchers (Dyomics) (e.g., DYQ-660 and DYQ-661); and ATTO fluorescent quenchers (ATTO-TEC GmbH) (e.g., ATTO 540Q, 580Q, and 612Q).

The term “detector,” as used herein, generally refers to a device that is capable of detecting a signal, including a signal indicative of the presence or absence of an incorporated nucleotide or nucleotide analog. In some cases, a detector can include optical and/or electronic components that can detect signals. The term “detector” may be used in detection methods. Non-limiting examples of detection methods include optical detection, spectroscopic detection, electrostatic detection, electrochemical detection, and the like. Optical detection methods include, but are not limited to, fluorimetry and UV-vis light absorbance. Spectroscopic detection methods include, but are not limited to, mass spectrometry, nuclear magnetic resonance (NMR) spectroscopy, and infrared spectroscopy. Electrostatic detection methods include, but are not limited to, gel based techniques, such as, for example, gel electrophoresis. Electrochemical detection methods include, but are not limited to, electrochemical detection of amplified product after high-performance liquid chromatography separation of the amplified products.

The term “adapter” or “adaptor,” as used herein, generally refers to a molecule (e.g., polynucleotide) that is adapted to permit a sequencing instrument to sequence a target polynucleotide, such as by interacting with a target nucleic acid molecule to facilitate sequencing (e.g., next generation sequencing (NGS)). The sequencing adapter may permit the target nucleic acid molecule to be sequenced by the sequencing instrument. For instance, the sequencing adapter may comprise a nucleotide sequence that hybridizes or binds to a capture polynucleotide attached to a solid support of a sequencing system, such as a bead or a flow cell. The sequencing adapter may comprise a nucleotide sequence that hybridizes or binds to a polynucleotide to generate a hairpin loop, which permits the target polynucleotide to be sequenced by a sequencing system. The sequencing adapter may include a sequencer motif, which may be a nucleotide sequence that is complementary to a flow cell sequence of another molecule (e.g., a polynucleotide) and usable by the sequencing system to sequence the target polynucleotide. The sequencer motif may also include a primer sequence for use in sequencing, such as sequencing by synthesis. The sequencer motif may include the sequence(s) for coupling a library adapter to a sequencing system and sequence the target polynucleotide (e.g., a sample nucleic acid). An adapter may comprise a barcode.

The term “barcode” or “barcode sequence,” as used herein, generally refers to one or more nucleotide sequences that may be used to identify one or more particular nucleic acids (e.g., based on their association with a particular sample, derivation from a particular source such as a particular cell, inclusion in a particular partition or other compartment, etc.). A barcode may comprise at least 1, 2, 3, 4, 5, 6, 7, 8, 9, 10, 11, 12, 13, 14, 15, 16, 17, 18, 19, 20, or more nucleotides (e.g., consecutive nucleotides). A barcode may comprise at least about 10, about 20, about 30, about 40, about 50, about 60, about 70, about 80, about 90, about 100 or more consecutive nucleotides. All of the barcodes used for an amplification and/or sequencing process (e.g., NGS) may be different. The diversity of different barcodes in a population of nucleic acids comprising barcodes may be randomly generated or non-randomly generated. For example, barcode sequences comprising multiple segments maybe assembled in a combinatorial fashion according to a split-pool scheme, in which a plurality of different first segments are distributed amongst a plurality of first partitions, the contents which are then pooled and distributed amongst a plurality of second partitions.

As described herein, the use of barcodes may permit high-throughput analysis of multiple samples using next generation sequencing techniques. A sample comprising a plurality of nucleic acid molecules may be distributed throughout a plurality of partitions (e.g., droplets in an emulsion), where each partition comprises a nucleic acid barcode molecule comprising a unique barcode sequence. The sample may be partitioned such that all or a majority of the partitions of the plurality of partitions include at least one nucleic acid molecule of the plurality of nucleic acid molecules. A nucleic acid molecule and nucleic acid barcode molecule of a given partition may then be used to generate one or more copies and/or complements of at least a sequence of the nucleic acid molecule (e.g., via nucleic acid amplification reactions), which copies and/or complements comprise the barcode sequence of the nucleic acid barcode molecule or a complement thereof. The contents of the various partitions (e.g., amplification products or derivatives thereof) may then be pooled and subjected to sequencing. In some cases, nucleic acid barcode molecules may be coupled to beads. In such cases, the copies and/or complements may also be coupled to the beads. Nucleic acid barcode molecules, and copies and/or complements may be released from the beads within the partitions or after pooling to facilitate nucleic acid sequencing using a sequencing instrument. Because copies and/or complements of the nucleic acid molecules of the plurality of nucleic acid molecules each include a unique barcode sequence or complement thereof, sequencing reads obtained using a nucleic acid sequencing assay may be associated with the nucleic acid molecule of the plurality of nucleic acid molecules to which they correspond. This method may be applied to nucleic acid molecules included within cells divided amongst a plurality of partitions, and/or nucleic acid molecules deriving from a plurality of different samples.

The terms “signal,” “signal sequence,” and “sequence signal,” as used herein, generally refer to a series of signals (e.g., fluorescence measurements) associated with a DNA molecule or clonal population of DNA, comprising primary data. Such signals may be obtained using a high-throughput sequencing technology (e.g., flow SBS). Such signals may be processed to obtain imputed sequences (e.g., during primary analysis).

The terms “sequence” or “sequence read,” as used herein, generally refer to a series of nucleotide assignments (e.g., by base calling) made during a sequencing process. Such sequences may be derived from signal sequences (e.g., during primary analysis).

The term “homopolymer,” as used herein, generally refers to a polymer or a portion of a polymer comprising identical monomer units, such as a sequence of 0, 1, 2, . . . , N sequential nucleotides. For example, a homopolymer containing sequential A nucleotides may be represented as A, AA, AAA, . . . , up to N sequential A nucleotides. A homopolymer may have a homopolymer sequence. A nucleic acid homopolymer may refer to a polynucleotide or an oligonucleotide comprising consecutive repetitions of a same nucleotide or any nucleotide variants thereof. For example, a homopolymer can be poly(dA), poly(dT), poly(dG), poly(dC), poly(rA), poly(U), poly(rG), or poly(rC). A homopolymer can be of any length. For example, the homopolymer can have a length of at least 2, 3, 4, 5, 10, 20, 30, 40, 50, 100, 200, 300, 400, 500, or more nucleic acid bases. The homopolymer can have from 10 to 500, or 15 to 200, or 20 to 150 nucleic acid bases. The homopolymer can have a length of at most 500, 400, 300, 200, 100, 50, 40, 30, 20, 10, 5, 4, 3, or 2 nucleic acid bases. A molecule, such as a nucleic acid molecule, can include one or more homopolymer portions and one or more non-homopolymer portions. The molecule may be entirely formed of a homopolymer, multiple homopolymers, or a combination of homopolymers and non-homopolymers. In nucleic acid sequencing, multiple nucleotides can be incorporated into a homopolymeric region of a nucleic acid strand. Such nucleotides may be non-terminated to permit incorporation of consecutive nucleotides (e.g., during a single nucleotide flow).

The term “HpN truncation,” as used herein, generally refers to a method of processing a set of one or more sequences such that each homopolymer of the set of one or more sequences having a length greater than or equal to an integer N is truncated to a homopolymer of length N. For example, HpN truncation of the sequence “AGGGGGT” to 3 bases may result in a truncated sequence of “AGGGT.”

The term “analog alignment,” as used herein, generally refers to alignment of signal sequences to a reference signal sequence.

The terms “amplifying,” “amplification,” and “nucleic acid amplification” are used interchangeably and, as used herein, generally refer to the production of copies of a nucleic acid molecule. For example, “amplification” of DNA generally refers to generating one or more copies of a DNA molecule. An amplicon may be a single-stranded or double-stranded nucleic acid molecule that is generated by an amplification procedure from a starting template nucleic acid molecule. Such an amplification procedure may include one or more cycles of an extension or ligation procedure. The amplicon may comprise a nucleic acid strand, of which at least a portion may be substantially identical or substantially complementary to at least a portion of the starting template. Where the starting template is a double-stranded nucleic acid molecule, an amplicon may comprise a nucleic acid strand that is substantially identical to at least a portion of one strand and is substantially complementary to at least a portion of either strand. The amplicon can be single-stranded or double-stranded irrespective of whether the initial template is single-stranded or double-stranded. Amplification of a nucleic acid may linear, exponential, or a combination thereof. Amplification may be emulsion based or may be non-emulsion based. Non-limiting examples of nucleic acid amplification methods include reverse transcription, primer extension, polymerase chain reaction (PCR), ligase chain reaction (LCR), helicase-dependent amplification, asymmetric amplification, rolling circle amplification, and multiple displacement amplification (MBA). An amplification reaction may be, for example, a polymerase chain reaction (PCR), such as an emulsion polymerase chain reaction (emPCR; e.g., PCR carried out within a microreactor such as a well or droplet). Where PCR is used, any form of PCR may be used, with non-limiting examples that include real-time PCR, allele-specific PCR, assembly PCR, asymmetric PCR, digital PCR, emulsion PCR, dial-out PCR, helicase-dependent PCR, nested PCR, hot start PCR, inverse PCR, methylation-specific PCR, miniprimer PCR, multiplex PCR, nested PCR, overlap-extension PCR, thermal asymmetric interlaced PCR and touchdown PCR. Moreover, amplification can be conducted in a reaction mixture comprising various components (e.g., a primer(s), template, nucleotides, a polymerase, buffer components, co-factors, etc.) that participate or facilitate amplification. In some cases, the reaction mixture comprises a buffer that permits context independent incorporation of nucleotides. Non-limiting examples include magnesium-ion, manganese-ion and isocitrate buffers. Additional examples of such buffers are described in Tabor, S. et al. C.C. PNAS, 1989, 86, 4076-4080 and U.S. Pat. Nos. 5,409,811 and 5,674,716, each of which is herein incorporated by reference in its entirety.

Amplification may be clonal amplification. The term “clonal,” as used herein, generally refers to a population of nucleic acids for which a substantial portion (e.g., greater than 50%, 60%, 70%, 80%, 90%, 95%, or 99%) of its members have substantially identical sequences (e.g., have sequences that are at least about 50%, 60%, 70%, 80%, 90%, 95%, or 99% identical to one another). Members of a clonal population of nucleic acid molecules may have sequence homology to one another. Such members may have sequence homology to a template nucleic acid molecule. In some instances, such members may have sequence homology to a complement of the template nucleic acid molecule (e.g., if single stranded). The members of the clonal population may be double stranded or single stranded. Members of a population may not be 100% identical or complementary because, e.g., “errors” may occur during the course of synthesis such that a minority of a given population may not have sequence homology with a majority of the population. For example, at least 50% of the members of a population may be substantially identical to each other or to a reference nucleic acid molecule (i.e., a molecule of defined sequence used as a basis for a sequence comparison). At least 60%, at least 70%, at least 80%, at least 90%, at least 95%, at least 99%, or more of the members of a population may be substantially identical to the reference nucleic acid molecule. Two molecules may be considered substantially identical (or homologous) if the percent identity between the two molecules is at least 60%, 70%, 75%, 80%, 85%, 90%, 95%, 98%, 99%, 99.9% or greater. Two molecules may be considered substantially complementary if the percent complementarity between the two molecules is at least 60%, 70%, 75%, 80%, 85%, 90%, 95%, 98%, 99%, 99.9% or greater. A low or insubstantial level of mixing of non-homologous nucleic acids may occur, and thus a clonal population may contain a minority of diverse nucleic acids (e.g., less than 30%, e.g., less than 10%).

Useful methods for clonal amplification from single molecules include rolling circle amplification (RCA) (Lizardi et al., Nat. Genet. 19:225-232 (1998), which is incorporated herein by reference), bridge PCR (Adams and Kron, Method for Performing Amplification of Nucleic Acid with Two Primers Bound to a Single Solid Support, Mosaic Technologies, Inc. (Winter Hill, Mass.); Whitehead Institute for Biomedical Research, Cambridge, Mass., (1997); Adessi et al., Nucl. Acids Res. 28:E87 (2000); Pemov et al., Nucl. Acids Res. 33:e11(2005); or U.S. Pat. No. 5,641,658, each of which is incorporated herein by reference), polony generation (Mitra et al., Proc. Natl. Acad. Sci. USA 100:5926-5931 (2003); Mitra et al., Anal. Biochem. 320:55-65(2003), each of which is incorporated herein by reference), and clonal amplification on beads using emulsions (Dressman et al., Proc. Natl. Acad. Sci. USA 100:8817-8822 (2003), which is incorporated herein by reference) or ligation to bead-based adapter libraries (Brenner et al., Nat. Biotechnol. 18:630-634 (2000); Brenner et al., Proc. Natl. Acad. Sci. USA 97:1665-1670 (2000)); Reinartz, et al., Brief Funct. Genomic Proteomic 1:95-104 (2002), each of which is incorporated herein by reference). The enhanced signal-to-noise ratio provided by clonal amplification more than outweighs the disadvantages of the cyclic sequencing requirement.

The term “context dependence” or “context dependency,” as used herein, generally refers to signal correlations with local sequence, relative nucleotide representation, or genomic locus. Signals for a given sequence may vary due to context dependency, which may depend on the local sequence, relative nucleotide representation of the sequence, or genomic locus of the sequence.

Flow sequencing by synthesis (SBS) may comprise performing repeated DNA extension cycles, wherein individual species of nucleotides and/or labeled analogs are presented to a primer-template-polymerase complex, which then incorporates the nucleotide if complementary. The product of each flow may be measured for each clonal population of templates, e.g., a bead or a colony. The resulting nucleotide incorporations may be detected and quantified by unambiguously distinguishing signals corresponding to or associated with zero, one, two, three, four, five, six, seven, eight, nine, ten, or more than ten sequential incorporations. Accurate quantification of such multiple sequential incorporations comprises quantifying characteristic signals for each possible homopolymer of 0, 1, 2, . . . , N sequential nucleotides incorporated on a colony in each flow. For example, a homopolymer containing sequential A nucleotides may be represented as A, AA, AAA, . . . , up to N sequential A nucleotides. Accurate quantification of homopolymer lengths (e.g., a number of sequential identical nucleotides in a sequence) may encounter challenges owing to random and unpredictable systematic variations in signal level, which can cause errors in quantifying the homopolymer length. In some cases, instrument and detection systematics can be calibrated and removed by monitoring instrument diagnostics and common-mode behavior across large numbers of colonies. Accurate quantification of homopolymer lengths (e.g., a number of sequential identical nucleotides in a sequence) may also encounter challenges owing to sequence context dependent signal, which may be different for every sequence. For example, in the case of fluorescence measurements of dilute labeled nucleotides, sequence context can affect both the number of labeled analogs (variable tolerance for incorporating labeled analogs) as well as fluorescence of individual labeled analogs (e.g., quantum yield of dyes affected by local context of ±5 bases, as described by [Kretschy, et al., Sequence-Dependent Fluorescence of Cy3- and Cy5-Labeled Double-Stranded DNA, Bioconjugate Chem., 27(3), pp. 840-848], which is incorporated herein by reference in its entirety). In practice, with dye-terminator Sanger cycle sequencing, substantial systematic variations in signals have been identified for 3-base contexts (e.g., as described by [Zakeri, et al., Peak height pattern in dichloro-rhodamine and energy transfer dye terminator sequencing, Biotechniques, 25(3), pp. 406-10], which is incorporated herein by reference in its entirety).

Generally, the nomenclature used herein and the laboratory procedures utilized in methods and systems of the present disclosure may include molecular, biochemical, microbiological and recombinant DNA techniques. Details of such techniques may be found in, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, C T (1994); Mishell and Shiigi (eds), “Strategies for Protein Purification and Characterization—A Laboratory Course Manual” CSHL Press (1996); all of which are incorporated by reference.

The term “trusted signal” or “trusted sequencing signal,” as used herein, generally refers to a sequencing signal that is an ideal signal, which is error free or at least a signal that is accurate enough to be trusted. The accuracy level may be determined in various manners. In some instances, a trusted signal may be a signal that meets a predetermined threshold for an accuracy level. A trusted sequencing signal may be used as a reference for generating a training set or for training an algorithm (e.g., a classifier such as a machine learning classifier). For example, a trusted sequencing signal may correspond to a known nucleotide sequence (e.g., a sequence of known bases), such that sets of trusted sequencing signals and sets of known nucleotide sequences may be used to construct training sets.

The goal of elucidating the entire human genome has created interest in technologies for rapid nucleic acid (e.g., DNA) sequencing, both for small and large scale applications. In addition, as knowledge of the genetic basis for human diseases increases, high-throughput DNA sequencing has been leveraged for myriad clinical applications. Despite the prevalence of nucleic acid sequencing methods and systems in a wide range of molecular biology and diagnostics applications, such methods and systems may encounter challenges in accurate base calling, such as when sequencing signals include regions of repeating nucleotide bases called homopolymers (‘h-mers’ or ‘hmers’). In particular, sequencing methods that perform base calling based on quantified characteristic signals indicating nucleotide incorporation can have sequencing errors (e.g., in quantifying homopolymer lengths), stemming from random and unpredictable systematic variations in signal levels and context dependent signals that may be different for every sequence. These signal errors can confound inherent sequencing errors from polymerase-based nucleotide incorporation. Such signal variations and context dependency signals may cause issues with sequence, especially homopolymer, calling.

Recognized herein is a need for improved base calling of sequences, and particularly sequences containing homopolymers. Methods and systems provided herein can significantly reduce or eliminate errors in base calling (e.g., related to quantifying homopolymer lengths and errors associated with context dependence). Such methods and systems may achieve accurate and efficient base calling of sequences (such as sequences containing homopolymers), quantification of homopolymer lengths, and quantification of context dependency in sequence signals.

Current methods of base calling may face significant challenges. For instance, coverage—the amount of duplicate signals obtained for each base in a sequence of interest—is an essential component of most error-reduction methods (i.e., higher coverage can increase the confidence in base calls). However, in cases in which a significant amount of reads cannot be used for training a base caller, for example due to read quality issues, coverage is unnecessarily decreases. Such challenges may arise because the base caller training may require as inputs a set of fully aligned reads that are of the same length. Any reads that have quality issues and have to be ‘trimmed’ may be thus excluded from the training set. Such exclusion of shorter reads may introduce undesirable bias into the training set toward long reads, which may be low copy, and can result in overfitting of the trained base caller.

The present disclosure provides methods and systems for improved base calling, in which training sets used for training a base caller (e.g., training a machine learning classifier such as a neural network) may be allowed to include reads of different lengths (e.g., thereby rescuing previously unusable reads). For example, a neural network that is trained for base calling may require input data of a fixed length in flow space (e.g., such that each read must include information for a same number of flows). Methods and systems of the present disclosure may comprise padding any “trimmed” reads with filler values (e.g., masked values), so that the training set may include a larger percentage of the total reads obtained during sequencing. For example, masking values may be negative numbers, whereby different negative values encode for or indicate a different class of trimmed flows (e.g., flows trimmed from reads for quality control metrics such as flow quality, 3Z, adapters, errors, variants, etc.). Masking values are described in more detail below.

In some embodiments, a set of sequencing reads may be processed by trimming at least a subset of the sequencing reads. In some embodiments the processing further comprises performing local alignment of at least a subset of the sequencing reads. In some embodiments, the processing further comprises performing adapter memorization of at least a portion of the sequencing reads. In some embodiments, the processing further comprises analyzing initial flows of at least a portion of the sequencing reads.

In some embodiments, sequencing reads may be trimmed based at least in part on a “3Z” code (e.g., indicative of 3 consecutive 0-signal flows). In flow-based sequencing, such as described herein, 3 consecutive flows resulting in no signals is not possible. Thus, 3Zs are indicative of errors in the sequencing, and reads including one more 3Zs may be trimmed to improve read quality and retain integrity of base calls. For example, in a given read with a “3Z” code, the first flow of the 3 consecutive 0-signal flows and all later flows in the read may be discarded from further consideration (e.g., for use in a base calling training set or other downstream analysis).

In some embodiments, reads may be trimmed based at least in part on a quality score. For example, all flows in a given read that fall below a pre-determined quality threshold may be discarded from further consideration (e.g., use in a base calling training set of other downstream analysis). In some instances, this may result in all flows beyond (i.e., downstream) of a quality drop being trimmed. In some embodiments, a quality score may be determined in accordance with different metrics. For example, each read may be encoded by a matrix with the dimensions n_hmers (h)×n_flows (f). A position (h, f) in such a matrix describes a probability h that the true base call for a flow (e.g., a corresponding to the read's flow f. Such a matrix may be referred to as a “flow matrix”. Qual string (QUAL) and the true positive (TP) tag may encode the columns of the flow matrix for non-zero flows (e.g., for flows where non-zero signals were received). Specifically, for an hmer=H, a number of error probabilities e are encoded min(4,floor((H+1)/2)) error probabilities. QUAL encodes values of the probabilities, and TP encodes the value of the error relatively to the called hmer (e.g., error h=3 if the called hmer is 4 may be encoded as -1).

Probabilities in QUAL may be expressed using Phred-encoding. For convenience, the errors may be encoded symmetrically relative to the middle of the hmer, with the nucleotide on either side of the hmer capturing half of the error probability. As an example, for P=(0,0,0,0.025,0.875,0.1,0), the hmer called is H=4; QUAL is “+11+”; and the value of tp is “+1,−1,−1,+1”. As another example, for P=(0,0,0.025,0.875,0.1,0), the hmer called is H=3; QUAL is “+.+” and the value of tp is “+1,−1,+1”.

In some embodiments, reads may be trimmed based at least in part on adapter trimming. For example, the adapter trimming may comprise removing or discarding any sequences that are recognized as an adapter sequence (e.g., a pre-determined adapter sequence).

In some embodiments, sequencing reads may be trimmed using one or more of the quality metrics defined herein.

The local alignment of reads may advantageously “rescue” some trimmed reads which would otherwise be discarded. In some embodiments, the local alignment of reads comprises adding masking values to reads for any flows that have been trimmed, thereby padding all reads to the same length. This local alignment approach may allow some mismatch for aligning, rather than requiring all aligned reads to have the same length. In some embodiments, the local alignment of reads is performed such that the largest segment of the read that is aligned predominates. In some embodiments, the local alignment of reads is performed such that the larger segment of the read that is aligned (e.g., Chimera reads) is selected and saved, with the remaining sequence masked. In some embodiments, the local alignment of reads is performed such that the if a middle portion of the read does not align, but the ends of the read do, then a read may be broken up into two sub-reads and separately aligned.

The local alignment of reads may advantageously serve as a replacement of Burrows-Wheeler alignment (BWA), which may be optimized for paired-end reads, with an aligner that functions in flow space (e.g., performing analog alignment of a set of flow signals to a set of reference flow signals) instead of base space (e.g., performing alignment of a string of nucleotide bases to a string of reference nucleotide bases). The flow-space aligner may have faster performance and/or improved variant calling as compared to a BWA aligner. In some embodiments, the flow-space aligner may be variant-aware (e.g., aligned such that a set of common variants is included). In some embodiments, the flow-space aligner may perform contamination detection (e.g., identify contamination from different genomes). In some embodiments, the flow-space aligner may feature re-defined mapping quality values (e.g., modified MapQ values for flow space).

The adapter memorization of reads may be performed in order to address issues with some reads being partially aligned while still including adapter sequences (e.g., such that the adapter sequence is mistakenly included as part of the genomic alignment), which makes it difficult to identify all adapter flows (e.g., even if 98% of adapter flows are identified, this can still cause issues downstream). In some embodiments, adapter memorization of reads may comprise manually inserting an indicator of a set of pre-determined (e.g., known) adapter sequences, which may depend on having knowledge of the adapter sequences used. For example, such adapters may be ligated onto one or both ends of nucleic acid molecules in order to facilitate nucleic acid sequencing (e.g., molecular barcoding, sample barcoding, etc.).

Analyzing initial flows may be performed, instead of excluding an initial set of flows (e.g., the first 1, 2, 3, 4, or 5 flows) from the training set due to uncertainty in calling the first base for the first h-mer of an insert.

The present disclosure may refer (for simplicity of explanation) to an E. coli genome, a human genome, a neural network and shotgun sequencing. These are examples of genomes of different sizes, machine learning processes, and a certain type of sequencing, respectively.

A detector may receive and output actual sequencing signals corresponding to fragments of human DNA, where the actual sequencing signals are subject to inaccuracies and noise. These inaccuracies and noise may be difficult or impossible to be analytically determined in advance (e.g., because they may be random). The present disclosure provides methods and systems that apply machine learning to assist in generating a mapping or classification between input datasets comprising actual human fragment sequencing signals (which may be noisy and inaccurate) and output datasets comprising accurate human fragment sequencing signals. The accurate human fragment sequencing signals may be further processed—for example, be aligned to an accurate human genome, for downstream applications, such as diagnostics and other precision health applications. By mapping actual signals more precisely to accurate signals, the method may serve to improve the overall quality of sequencing and hence the quality of diagnoses and treatments based at least in part on such sequencing.

The human genome comprises over three billion base pairs. Such a large genome, in some instances, presents challenges in generating a direct mapping between a set of actual human fragment sequencing signals (which may be noisy and inaccurate) and a set of accurate human fragment sequencing signals. The present disclosure provides methods and systems of first applying a machine learning process to much smaller genomes—for example on an E. coli genome that comprises approximately three thousand genes (i.e., approximately four million base pairs)—and then applying the machine learning process to larger genomes (e.g., a human genome). Such methods make direct mapping between a set of actual human fragment sequencing signals and a set of accurate human fragment sequencing signals more feasible (e.g., by pre-training a machine learning classifier on a smaller genome prior to updating or retraining the machine learning classifier for a larger genome). Although the E. coli genome differs significantly from the human genome, it may be used during a multi-phase training process that comprises one or more of the following: (a) obtaining a first trained algorithm (e.g., a machine learning process) comprising a first mapping (e.g., classification or regression) between actual reference sequencing signals and trusted reference sequencing signals; (b) obtaining actual sequencing signals corresponding to the second genome; and (c) generating a training set for training a second trained algorithm (e.g., machine learning process) comprising a second mapping (e.g., classification or regression) between actual sequencing signals corresponding to the second genome and trusted sequencing signals corresponding to the second genome.

In some embodiments, the actual reference sequencing signals and the trusted reference sequencing signals each represent regions of a reference genome (e.g., one or more sections less than the whole reference genome). In some embodiments, the reference genome is of a first genus that differs from a second genome of a second genus. In some embodiments, the reference genome is smaller than the second genome. In some embodiments, the training set is generated based on the first mapping with the actual sequencing signals corresponding to the second genome. In some embodiments, the multi-phase process further comprises generating the second mapping using one or more machine learning processes that are of reasonable complexity and cost.

It will be appreciated that while the present disclosure is explained with respect to correlating and/or mapping, for example, the human genome and E. coli genome with various training algorithms, the methods and systems of the present disclosure may be applicable to any two genomes, such as where one genome is bigger and/or more complex than the other genome. For example, actual sequencing signals of a non-human sample (i.e., from a subject of a third genus) may be received or generated.

The present disclosure provides systems, methods, and computer-readable media that generate a second mapping based on a first mapping corresponding to a genus having a genome that is smaller than the human genome (e.g., the E. coli genome). The second mapping can be used to process actual human fragment sequencing signals to produce accurate human fragment sequencing signals, which may be aligned to a reference human genome in order to provide an estimate of the genome of a subject.

The method may comprise obtaining or generating a first trained algorithm comprising a first mapping between reference actual sequencing signals and reference trusted sequencing signals (e.g., between actual E. coli fragment sequencing signals and accurate E. coli fragment sequencing signals). The second trained algorithm configured to apply the second mapping may be trained using a machine learning process.

Machine learning processes suitable for use with methods described herein may comprise (i) using a first trained algorithm (e.g., a first neural network) that is trained to apply the first mapping to process actual E. coli fragment sequencing signals to produce accurate E. coli fragment sequencing signals, and (ii) using a second trained algorithm (e.g., a second neural network) that is trained to apply the second mapping to process actual human fragment sequencing signals to produce accurate human fragment sequencing signals. The accurate human fragment sequencing signals may then be aligned to a reference human genome (e.g., for further genomic analysis).

The first trained algorithm may generate a training set (e.g., training dataset) that may be used to train a second trained algorithm (e.g., a second neural network) to apply a second mapping between actual sequencing signals and accurate sequencing signals corresponding to a human genome (e.g., between actual human fragment sequencing signals and accurate human fragment sequencing signals).

The systems, methods, and computer-readable media may be highly efficient in terms of memory and/or computational resources, as they are configured to apply machine learning algorithms on the E. coli genome (or any other small genome that is much smaller than the human genome). Therefore, such systems, methods, and computer-readable media may advantageously perform sequence calling or base calling with greater accuracy and efficiency, while using less memory and/or computational resources.

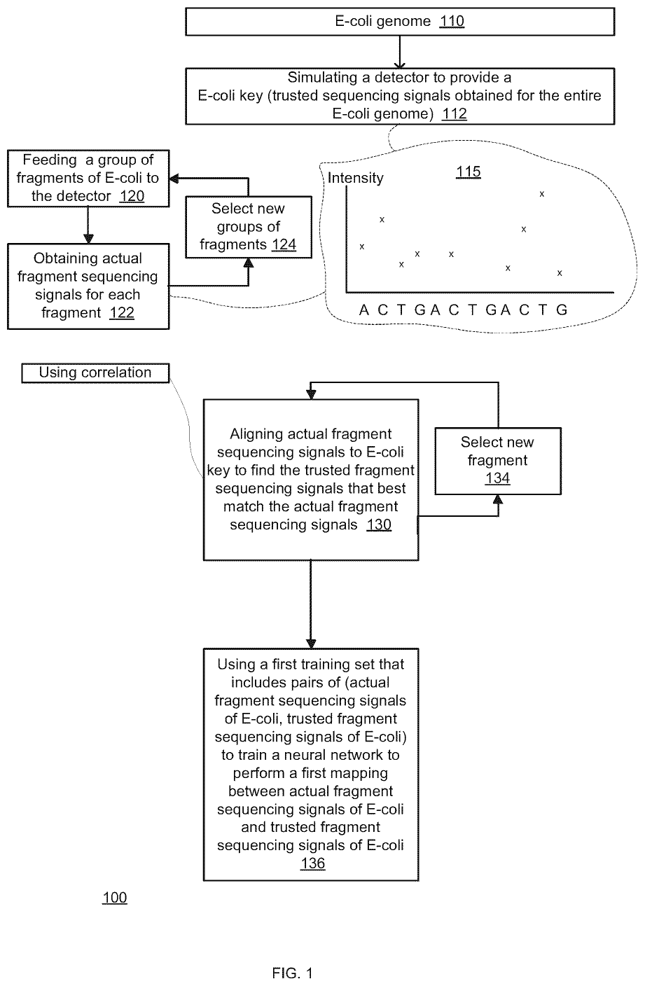

FIG. 1 shows an example of a method 100 for training a neural network configured to apply a first mapping between actual fragment sequencing signals of E. coli and trusted fragment sequencing signals of E. coli. In some embodiments, method 100 may include one or more of operations 110, 112, 120, 122, 124, 130, 134, and 136.

The method 100 may comprise receiving a genome corresponding to a genus or a species (e.g., an E. coli genome) that differs from the human genome (as in operation 110). For example, the E. coli genome may comprise about 4.6 million base pairs, which is significant smaller than the human genome (which may comprise about 3 billion base pairs). The use of a smaller genome may be advantageous to reduce computational complexity (thereby enabling faster runtimes with less computational resources), which may scale linearly with the size of the genome.

In some embodiments, the method 100 may further comprise simulating a detector (e.g., especially simulating the response of the detector to the E. coli genome)—assuming a substantially error-free process (as in operation 112). The method 100 may comprise simulating the chemical and/or optical processes executed by the detector (as in operation 112). The outcome of operation 112 may be an E. coli key (115) which includes trusted sequencing signals that may be expected to be obtained from the detector (under a substantially error-free detection process) for the entire E. coli genome. The E. coli key 115 may include intensity values for A, C, T, G elements for the entire E. coli genome.

In some embodiments, the method 100 may further comprise processing a group of fragments of E. coli nucleic acid samples using the detector (as in operation 120). In some embodiments, the method 100 may further comprise obtaining actual fragment sequencing signals for each segment (as in operation 122). In some embodiments, the method 100 may further comprise selecting a new group of fragments (as in operation 124) and proceeding to operation 120. The set of operations 120, 122, and 124 may be repeated or iterated until receiving actual fragment sequencing signals for the entire E. coli genome, or until a substantial amount of actual fragment sequencing signals are received.

In some embodiments, operation 122 may further comprise rejecting actual fragment sequencing signals that may be defective (e.g., based on one or more quality metrics). For example, while ideal, noise-free fragment sequencing signals may be expected to represent an integer number of homopolymers, the actual fragment sequencing signals may provide a non-integer number of homopolymers. The deviation from the expected integer numbers of homopolymers may be indicative of an error in the actual fragment sequencing signals, and once the error exceeds a predefined threshold, the actual fragment sequencing signals may be ignored and may not be processed in subsequent operations, such as operations 130 and 136. The error may be calculated in various manners, for example, mean squared error, and the like. The predefined threshold may be set in any manner.

In some embodiments, the method 100 may further comprise aligning actual fragment sequencing signals to the E. coli key 115 (as in operation 130). Operation 130 may comprise correlating the actual fragment sequencing signals against the entire E. coli key to find the location of the best matching trusted fragment sequencing signals in the E. coli key.

In some embodiments, the method 100 may further comprise selecting a new group of fragments (as in operation 134) and proceeding to operation 130. The set of operations 130 and 134 may be repeated or iterated until finding, for each one of the actual fragment sequencing signals, best matching trusted fragment sequencing signals in the E. coli key. In some instances, substantially all of the actual fragment sequencing signals may be matched to trusted fragment sequencing signals. In some instances, all of the actual fragment sequencing signals may be matched to trusted fragment sequencing signals. In some instances, any percentage, such as at least 10%, 20%, 30%, 40%, 50%, 60%, 70%, 80%, 90%, 95%, 96%, 97%, 98%, 99% or more of the set of actual fragment sequencing signals may be matched to trusted fragment sequencing signals. In some embodiments, the pairs, or array or pairs, of actual fragment sequencing signals and the best matching trusted fragment sequencing signals in the E. coli key (for the actual fragment sequencing signals) may form a first training set.

In some embodiments, the method 100 may further comprise using the first training set that includes pairs of actual fragment sequencing signals of E. coli, and trusted fragment sequencing signals of E. coli to train a neural network to perform a first mapping (e.g., classification or regression) between actual fragment sequencing signals of E. coli and trusted fragment sequencing signals of E. coli (as in operation 136).

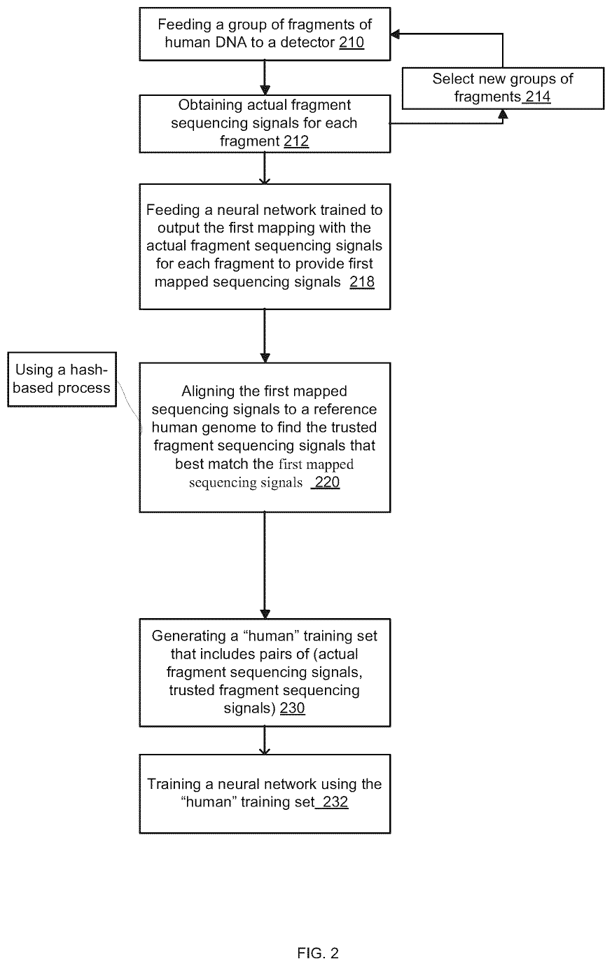

FIG. 2 shows an example of a method 200 for using a neural network (trained to apply the first mapping) for generating a second training set that may be used to map actual fragment sequencing signals of a certain person to trusted fragment sequencing signals of a reference human genome.

The method 200 may comprise processing a group of fragments of a human DNA using a detector (as in operation 210). For example, the operation 210 may comprise using a known human DNA of known variants and either ignoring the variants or compensating for the variants. In some embodiments, the method 200 may further comprise obtaining actual fragment sequencing signals for each segment (as in operation 212). These actual fragment sequencing signals may be the outputs of the detector.

In some embodiments, the method 200 may further comprise selecting a new group of fragments (as in operation 214) and proceeding to operation 210. The set of operations 210, 212, and 214 may be repeated or iterated until receiving actual fragment sequencing signals for the entire human genome, or until a substantial amount of actual fragment sequencing signals are received. In some embodiments, operation 212 may further comprise rejecting actual fragment sequencing signals that may be defective. For example, while noise-free fragment sequencing signals may be expected to represent an integer number of homopolymers, the actual fragment sequencing signals may provide a non-integer number of homopolymers. The deviation from the expected integer numbers of homopolymers may be indicative of an error in the actual fragment sequencing signals, and once the error exceeds a predefined threshold, the actual fragment sequencing signals may be ignored and may not be processed in operations 218 and 220. The error may be calculated in various manners, for example, mean squared error, and the like. The predefined threshold may be set in any manner.

In some embodiments, the method 200 may further comprise using a neural network trained to output the first mapping to process the actual fragment sequencing signals for each fragment to provide first mapped sequencing signals (as in operation 218).

In some embodiments, the method 200 may further comprise aligning the first mapped sequencing signals to a reference human genome to determine the trusted fragment sequencing signals that best match the first mapped sequencing signals (as in operation 220). These trusted fragment sequencing signals may be regarded as best matching the actual fragment sequencing signals. The method 200 may comprise repeating operations 218 and 220 for each of the actual fragment sequencing signals provided in operation 212. In some instances, substantially all of the first mapped sequencing signals may be matched to trusted fragment sequencing signals. In some instances, all of the first mapped fragment sequencing signals may be matched to trusted fragment sequencing signals. In some instances, any percentage, such as at least 10%, 20%, 30%, 40%, 50%, 60%, 70%, 80%, 90%, 95%, 96%, 97%, 98%, 99% or more of the set of first mapped fragment sequencing signals may be matched to trusted fragment sequencing signals.

In some embodiments, the method 200 may further comprise generating a “human” training set that includes pairs of actual fragment sequencing signals, and trusted fragment sequencing signals that correspond to the human genome (as in operation 230). In some embodiments, the method 200 may further comprise training a neural network using the “human” training set (as in operation 232). After the training, the neural network is configured apply a second mapping (e.g., classification or regression) between actual fragment sequencing signals corresponding to the human genome and trusted fragment sequencing signals corresponding to the human genome.

Using systems, methods, and media of the present disclosure, a more robust method may be provided when using truncated actual human sequencing signals and truncated trusted reference sequencing signals. Truncating these signals, such as to single-bit actual human sequencing signals and single-bit trusted reference sequencing, may provide a method that is robust to measurement error, while incurring a tolerable cost of finding more candidates for each hash value during the alignment procedure. After the completion of methods 100 and 200, an estimate of a genome of a subject may be generated.

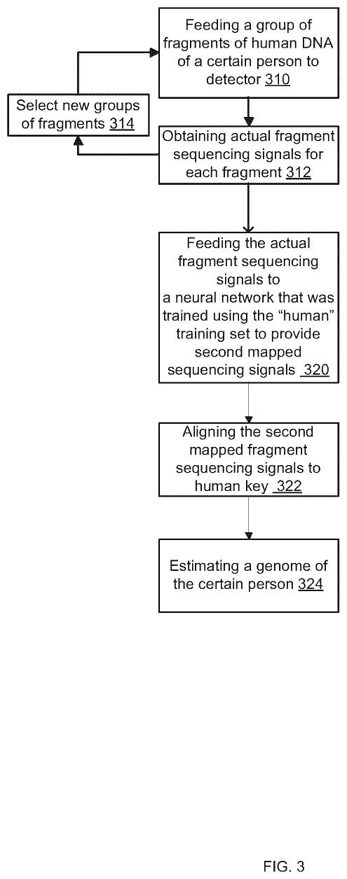

FIG. 3 shows an example of a method 300 for estimating a genome of a subject.

The method 300 may comprise processing a group of fragments of a human DNA of the subject using the detector. In some embodiments, the method 300 may comprise obtaining actual fragment sequencing signals for each segment (as in operation 312). In some embodiments, operation 312 may further comprise assigning a confidence level to actual fragment sequencing signals. For example, while noise-free fragment sequencing signals may be expected to represent an integer number of homopolymers, the actual fragment sequencing signals may provide a non-integer number of homopolymer. The deviation from the expected integer numbers of homopolymers may be indicative of an error in the actual fragment sequencing signals, that may affect the confidence level assigned to the actual fragment sequencing signals.

In some embodiments, the method 300 may further comprise selecting new group of fragments (as in operation 314) and proceeding to operation 310. The set of operations 310, 312, and 314 may be repeated or iterated until receiving actual fragment sequencing signals for the entire genome of the subject, or until a substantial amount of actual fragment sequencing signals are received. In some embodiments, the method 300 may comprise repeating operations 320 and 322 for each of the actual fragment sequencing signals provided in operation 312.

In some embodiments, operation 320 may comprise processing the actual fragment sequencing signals using a neural network that is trained using the “human” training set to provide second mapped sequencing signals. In some embodiments, method 300 may further comprise aligning the second mapped fragment sequencing signals to a human key (as in operation 322). For example, the alignment may be hash-based. In some embodiments, one or more iterations of operation 322 may further comprise providing an estimate of the genome of the subject (as in operation 324).

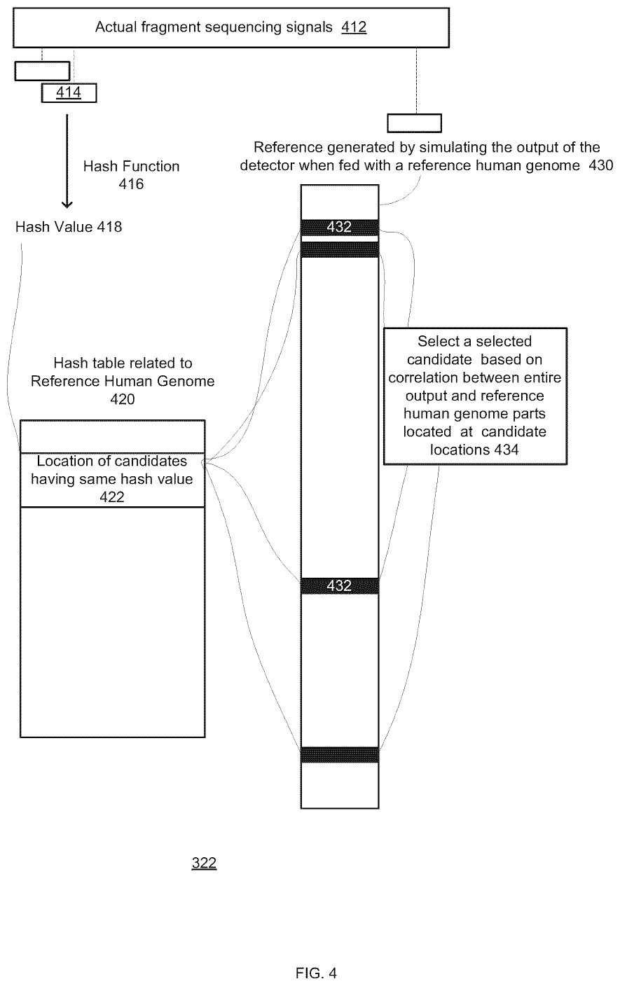

FIG. 4 shows an example of a method 400 for hash-based alignment (e.g., according to operation 322).

The method 400 may comprise partitioning actual fragment sequencing signals 412 into smaller partially overlapping portions 414, in order to simplify the execution of operation 322. For example, actual fragment sequencing signals 412, where each actual fragment sequencing signal comprises about one hundred values, may be partitioned into portions 414, each of which may comprise about twenty values.

In some instances, the method 400 may further comprise applying a hash function 416 on each portion 414 to provide a hash value 418. In some embodiments, the hash value 418 is used as an index to a hash table 420 corresponding to a reference human genome.

An entry of the hash table 420 that is accessed by a certain hash value may store the locations of candidates (e.g., those that have the same hash value) in a data structure, which stores a reference database 430. In some instances, the reference database 430 is generated by simulating the output of the detector from processing a reference human genome. The simulation may assume a substantially error-free process.

In some embodiments, method 400 may further comprise using hash value 418 to access entry 422, which stores locations of candidates (432) in the reference database 430. In some embodiments, the different references are associated with different locations in the reference human genome. In order to select the selected candidate, a correlation (434) between the actual fragment sequencing signals (412) and portions of the reference (430) located at each of the different locations is determined. The selection may include selecting the location with the highest correlation.

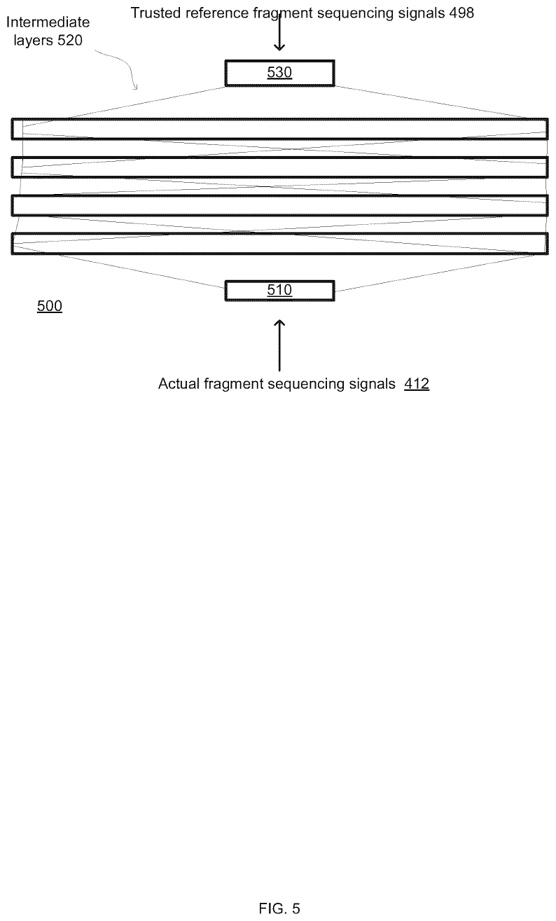

FIG. 5 shows an example neural network 500 that may be trained during method 100 and/or method 200. In some instance, neural network 500 may be used in performing method 300.

In some instances, the neural network 500 may include an input layer 510, a plurality of intermediate layers 520, and an output layer 530. In some embodiments, neural network 500 is a regression network such as a fully connected regression network.

In some embodiments, the input layer 510 may include one neuron per actual fragment sequencing signal. For example, if the input layer is fed by actual fragment sequencing signals of one hundred values, then the input layer 510 may include one hundred neurons. A similar example may apply to the output layer. Each intermediate layer may be much larger than the input layer. For example, an intermediate layer may be about 1.5×, 2×, 3×, 4×, 5×, 6×, 7×, 8×, 9×, 10×, or more than 10× larger than the input layer. Other ratios may be used.

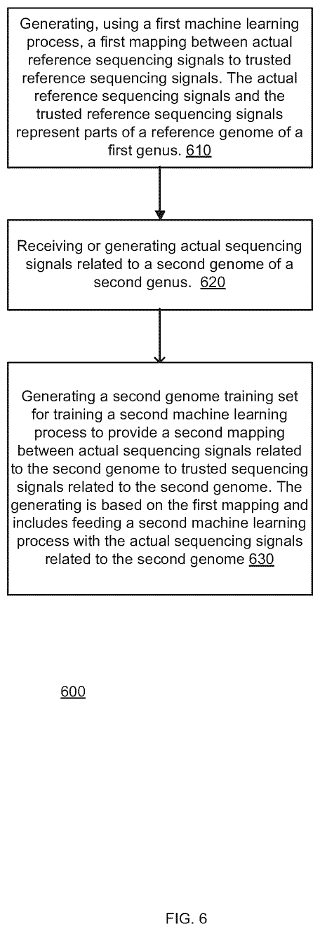

FIG. 6 shows an example of a method 600 for generating a training set.

The method 600 may comprise generating, using a first trained algorithm (e.g., a machine learning process), a first mapping (e.g., classification or regression) between actual reference sequencing signals to trusted reference sequencing signals. The actual reference sequencing signals and the trusted reference sequencing signals may represent regions of a reference genome of a first genus (e.g., a human genome).

In some embodiments, the method 600 may further comprise applying the operations of method 100 on a first genome (e.g., a human genome) of a first genus that may differ from E. coli. In some embodiments, method 600 may further comprise receiving or generating actual sequencing signals corresponding to a second genome of a second genus (as in operation 620). The first genus may differ from the second genus. The first genome may be smaller than the second genome, for example, by a factor of at least about 2, 3, 4, 5, 6, 7, 8, 9, 10, 15, 20, 25, 30, 35, 40, 45, 50, 55, 60, 65, 70, 75, 80, 85, 90, 95, 100, 200, 300, 400, 500, 600, 700, 800, 900, 1000, 2000, 3000, 4000, 5000, 6000, 7000, 8000, 9000, or 10000. Other factors may be applied.

In some embodiments, method 600 may further comprise generating a second genome training set for training a second trained algorithm (e.g., machine learning process) to provide a second mapping (e.g., classification or regression) between actual sequencing signals corresponding to the second genome to trusted sequencing signals corresponding to the second genome (as in operation 630).

In some embodiments, operation 630 may be performed based on the first mapping, and may include using a second trained algorithm (e.g., machine learning process) to process the actual sequencing signals corresponding to the second genome. In some embodiments, operation 630 may apply the operations of method 200 on a second genome of a second genus that may differ from human (e.g., E. coli). In some embodiments, operation 630 may be followed by training a trained algorithm (e.g., machine learning process) using the second genome training set.

In some embodiments, the first trained algorithm (e.g., machine learning process) may differ from the second trained algorithm (e.g., another machine learning process) or may be the same as the second trained algorithm.

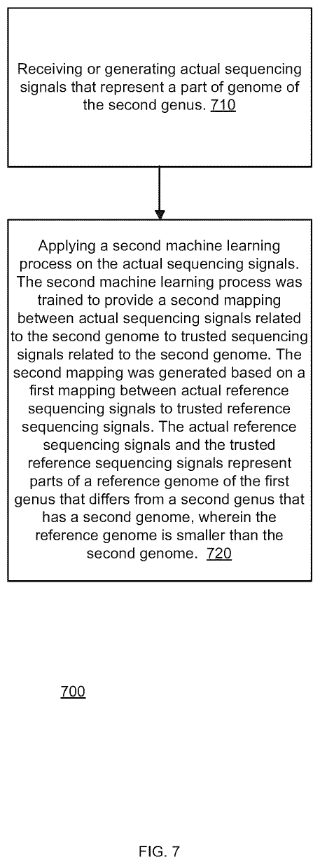

FIG. 7 shows an example of a method 700 for estimating a genome of a subject of a second genus. The estimation may be performed based on a first genus, and method 700 may be referred to as a method for first genus-based estimation of a genome of a second genus.

The method 700 may comprise performing operations 710 and 720 for each part of the genome of the subject of the second genus, out of multiple regions of the genome of the second genus. The method 700 may comprise performing one or more repetitions or iterations of the set of operations 710 and 720 to provide the estimate of the genome of the subject of the second genus.

In some embodiments, operation 710 may further comprise receiving or generating actual sequencing signals that represent a part of genome of the second genus. In some embodiments, operation 720 may further comprise estimating the part of the genome of the subject of the second genus.

In some embodiments, operation 720 may further comprise applying a second trained algorithm (e.g., machine learning process) to the actual sequencing signals. The second trained algorithm (e.g., another machine learning process) may be trained to provide a second mapping (e.g., classification or regression) between actual sequencing signals corresponding to the second genome and trusted sequencing signals corresponding to the second genome. The second mapping may be generated based on a first mapping between actual reference sequencing signals and trusted reference sequencing signals. The actual reference sequencing signals and the trusted reference sequencing signals may represent regions of a reference genome of the first genus that differ from a second genome of a second genus. The reference genome may be smaller than the second genome.

In some embodiments, operations 710 and 720 may further comprise applying the operations of method 300 on a second genus that may differ from human, wherein the first mapping may relate to a first genus other than E. coli.

### Trained Algorithms

After processing biological samples to generate sequencing signals of nucleic acids, a trained algorithm may be used to process the sequencing signals to perform sequencing calling (e.g., determining the base calls based on the sequence signals). For example, the trained algorithm may be used to determine quantitative measures of sequence signals at each of a plurality of nucleotide positions of the nucleic acids. The trained algorithm may be configured to determine the quantitative measures of the sequence signals an accuracy of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or more than 99%.

The trained algorithm may comprise a supervised machine learning algorithm. The trained algorithm may comprise a classification and regression tree (CART) algorithm. The supervised machine learning algorithm may comprise, for example, a Random Forest, a support vector machine (SVM), a neural network, or a deep learning algorithm. The trained algorithm may comprise an unsupervised machine learning algorithm.

The trained algorithm may be configured to accept a plurality of input variables and to produce one or more output values based on the plurality of input variables. The plurality of input variables may be generated based on processing sequencing signals of nucleic acids. For example, an input variable may comprise a number of sequences corresponding to or aligning to a reference genome or genomic loci of a reference genome. As another example, an input variable may comprise analog values of sequencing signals produced by a sequencer.

The trained algorithm may comprise a classifier, such that each of the one or more output values comprises one of a fixed number of possible values (e.g., a linear classifier, a logistic regression classifier, etc.) indicating a classification of the sequencing signals by the classifier. The trained algorithm may comprise a binary classifier, such that each of the one or more output values comprises one of two values (e.g., {0, 1}, {positive, negative}, or {present, absent}) indicating a classification of the sequencing signals by the classifier. The trained algorithm may be another type of classifier, such that each of the one or more output values comprises one of more than two values (e.g., {0, 1, 2}, {positive, negative, or indeterminate}, {present, absent, or indeterminate}, {A, C, G, T}, or {A, C, G, U}) indicating a classification of the sequencing signals by the classifier. The output values may comprise descriptive labels, numerical values, or a combination thereof. Some of the output values may comprise descriptive labels. Such descriptive labels may provide an identification of base calls of the sequence signals, and may comprise, for example, {A, C, G, T}, or {A, C, G, U}. Such descriptive labels may provide an indication of context for a base call, or a confidence or accuracy for a base call. As another example, such descriptive labels may provide a relative assessment of the likelihood of different bases being called for the sequencing signals. Some descriptive labels may be mapped to numerical values, for example, by mapping “positive” or “present” to 1, and “negative” or “absent” to 0.

Some of the output values may comprise numerical values, such as binary, integer, or continuous values. Such binary output values may comprise, for example, {0, 1}, {positive, negative}, or {present, absent}. Such integer output values may comprise, for example, {0, 1, 2}. Such continuous output values may comprise, for example, a probability value of at least 0 and no more than 1 (e.g., indicative of the likelihood of a base call for a sequencing signal). Such continuous output values may comprise, for example, an un-normalized probability value of at least 0. Some numerical values may be mapped to descriptive labels, for example, by mapping 1 to “positive” or “present”, and 0 to “negative” or “absent”.

Some of the output values may be assigned based on one or more cutoff values. For example, a binary classification of sequencing signals may assign an output value of “positive” or 1 if the sequencing signal at a particular nucleotide position has at least a 50% probability of being called as a given base (e.g., A, C, G, T, or U). For example, a binary classification of samples may assign an output value of “negative” or 0 if the sequencing signal at a particular nucleotide position has at least a 50% probability of being called as a given base (e.g., A, C, G, T, or U). In this case, a single cutoff value of 50% is used to classify bases of sequencing signals into one of the two possible binary output values. Examples of single cutoff values may include about 1%, about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, and about 99%.

As another example, a classification of sequencing signals may assign an output value of “positive” or 1 if the sequencing signal at a particular nucleotide position has a probability of being called as a given base (e.g., A, C, G, T, or U) of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more. The classification of sequencing signals may assign an output value of “positive” or 1 if the sequencing signal at a particular nucleotide position has a probability of being called as a given base (e.g., A, C, G, T, or U) of more than about 50%, more than about 55%, more than about 60%, more than about 65%, more than about 70%, more than about 75%, more than about 80%, more than about 85%, more than about 90%, more than about 91%, more than about 92%, more than about 93%, more than about 94%, more than about 95%, more than about 96%, more than about 97%, more than about 98%, or more than about 99%.

The classification of sequencing signals may assign an output value of “negative” or 0 if the sequencing signal at a particular nucleotide position has a probability of being called as a given base (e.g., A, C, G, T, or U) of less than about 50%, less than about 45%, less than about 40%, less than about 35%, less than about 30%, less than about 25%, less than about 20%, less than about 15%, less than about 10%, less than about 9%, less than about 8%, less than about 7%, less than about 6%, less than about 5%, less than about 4%, less than about 3%, less than about 2%, or less than about 1%. The classification of sequencing signals may assign an output value of “negative” or 0 if the sequencing signal at a particular nucleotide position has a probability of being called as a given base (e.g., A, C, G, T, or U) of no more than about 50%, no more than about 45%, no more than about 40%, no more than about 35%, no more than about 30%, no more than about 25%, no more than about 20%, no more than about 15%, no more than about 10%, no more than about 9%, no more than about 8%, no more than about 7%, no more than about 6%, no more than about 5%, no more than about 4%, no more than about 3%, no more than about 2%, or no more than about 1%.

The classification of sequencing signals may assign an output value of “indeterminate” or 2 if the sample is not classified as “positive”, “negative”, 1, or 0. In this case, a set of two cutoff values is used to classify sequencing signals into one of the three possible output values. Examples of sets of cutoff values may include {1%, 99%}, {2%, 98%}, {5%, 95%}, {10%, 90%}, {15%, 85%}, {20%, 80%}, {25%, 75%}, {30%, 70%}, {35%, 65%}, {40%, 60%}, and {45%, 55%}. Similarly, sets of n cutoff values may be used to classify sequencing signals into one of n+1 possible output values, where n is any positive integer.

The trained algorithm may be trained with a plurality of independent training samples. Each of the independent training samples may comprise sets of sequencing signals generated from nucleic acids (e.g., from biological sample of a subject) and one or more known output values corresponding to the sequencing signals (e.g., a set of base calls or a nucleotide sequence corresponding to the sequencing signals). Independent training samples may be obtained or derived from a plurality of different subjects. Independent training samples may comprise sets of sequencing signals generated from nucleic acids (e.g., from biological sample of a subject) and one or more known output values corresponding to the sequencing signals (e.g., a set of base calls or a nucleotide sequence corresponding to the sequencing signals) obtained at a plurality of different time points from the same subject (e.g., on a regular basis such as weekly, biweekly, or monthly).

The trained algorithm may be trained with at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, or at least about 500 independent training samples. The trained algorithm may be trained with no more than about 500, no more than about 450, no more than about 400, no more than about 350, no more than about 300, no more than about 250, no more than about 200, no more than about 150, no more than about 100, or no more than about 50 independent training samples.

The trained algorithm may be configured to identify base calls of the sequencing signals at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more than about 99%. The accuracy of identifying the base calls of the sequencing signals by the trained algorithm may be calculated as the percentage of base calls that are correctly identified or classified (e.g., presence or absence of a particular base).

The trained algorithm may be configured to identify base calls of the sequencing signals with a positive predictive value (PPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The PPV of identifying the base calls of the sequencing signals using the trained algorithm may be calculated as the percentage of base calls identified or classified as being present that correspond to bases that are truly present.

The trained algorithm may be configured to identify base calls of the sequencing signals with a negative predictive value (NPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The NPV of identifying the base calls of the sequencing signals using the trained algorithm may be calculated as the percentage of base calls identified or classified as being absent that correspond to bases that are truly absent (e.g., not present).

The trained algorithm may be adjusted or tuned to improve one or more of the performance, accuracy, PPV, or NPV of identifying the base calls of the sequencing signals. The trained algorithm may be adjusted or tuned by adjusting parameters of the trained algorithm (e.g., a set of cutoff values used to identify base calls of sequencing signals, as described elsewhere herein, or weights of a neural network). The trained algorithm may be adjusted or tuned continuously during the training process or after the training process has completed.

After the trained algorithm is initially trained, a subset of the inputs may be identified as most influential or most important to be included for making high-quality classifications. The plurality of input variables or a subset thereof may be ranked based on classification metrics indicative of each input variable's importance toward making high-quality classifications or identifications of base calls of sequencing signals. Such metrics may be used to reduce, in some cases significantly, the number of input variables (e.g., predictor variables) that may be used to train the trained algorithm to a desired performance level (e.g., based on a desired minimum accuracy, PPV, or NPV, or a combination thereof). For example, if training the trained algorithm with a plurality comprising several dozen or hundreds of input variables in the trained algorithm results in an accuracy of classification of more than 99%, then training the trained algorithm instead with only a selected subset of no more than about 5, no more than about 10, no more than about 15, no more than about 20, no more than about 25, no more than about 30, no more than about 35, no more than about 40, no more than about 45, no more than about 50, or no more than about 100 such most influential or most important input variables among the plurality can yield decreased but still acceptable accuracy of classification (e.g., at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%). The subset may be selected by rank-ordering the entire plurality of input variables and selecting a predetermined number (e.g., no more than about 5, no more than about 10, no more than about 15, no more than about 20, no more than about 25, no more than about 30, no more than about 35, no more than about 40, no more than about 45, no more than about 50, or no more than about 100) of input variables with the best classification metrics.

The present disclosure provides computer systems that are programmed to implement methods of the disclosure. In some embodiments, a neural network used to implement method 100 and/or method 200 may be a U-Net.

U-Net is a convolutional neural network that was developed for biomedical image segmentation at the Computer Science Department of the University of Freiburg, Germany. The network may be based on the fully convolutional neural network, and its architecture is modified and extended to work with fewer training images and to yield more precise segmentations. For example, segmentation of a 512×512 image may be performed using a U-Net in less than a second on a modern GPU.

The U-net may be a combination of two deep learning methods: a convolutional neural network (CNN) and an Encoder—Decoder. The CNN may be configured to handle large input images with a relatively small number of weights in the network. This is possible because the input image is typically position invariant—the filter operated in one section of the input image is the same as those in other sections of the input image. Therefore, the CNN applies the same filters in all parts of the input image, thereby allowing optimization with a reasonable number of parameters, and achieving the machine learning process to be performed with a manageable number of samples in a reasonable time. The encoder -decoder is a method for performing dimensionality reduction in a machine learning process. It may comprise having a network map all the input variables to a small number of weights, and decoding the weights back to the input image. This technique enables using information from the entire input image with a small number of parameters.

The U-Net may use both the CNN and encoder-decoder techniques in parallel, thereby allowing for repeated reuse of the same filter in the input image and considering large scale effect of the image.

Methods, systems, and media of the present disclosure may perform the processing of actual human fragment sequencing signals in a similar manner as that used for Semantic Segmentation, by leveraging some parallel elements.

In some embodiments, actual human fragment sequencing signals may be treated as a single dimension (1D) image. Both input images and actual human fragment sequencing signals may exhibit the property of having most of the information be flow invariant—as the sequence calling or base calling of the actual human fragment sequencing signals may comprise analysis of the values of the actual human fragment sequencing signals and on the immediate surrounding values of the actual human fragment sequencing signals. Nevertheless, the processing of the actual human fragment sequencing signals may also use information from the entire read, therefore using the encoder part of the network may be beneficial.

The U-Net may be fed by various types of information. The different types of information can be seen as different information channels. For example, the different information types may include the actual human fragment sequencing signals and may also include one or more other additional types of information. As an example, an additional type of information may include calculation of the photometry background noise, which was found to be beneficial information.

As another example, an additional type of information may include the sequencing signals obtained from the preamble. The preamble may be attached to the tested human genome fragments, and may be known in advance. The sequencing signals obtained from the preamble may be expected to be substantially the same for all reads. The intensity of the sequencing signals obtained from the preamble may be indicative of an approximation of the number of strands in the bead. It can be useful in a normalization of the sequencing signals obtained from the preamble.

As another example, an additional type of information may include local information corresponding to the vicinity of the readings. For example, the local information may represent readings with a tile, such as a reading per flow. A substrate that supports the samples may be virtually segmented to tiles (for example, tents till thousands of tiles), and the local information may reflect readings corresponding to a given tile. For example, the readings may be calculated as a mean signal for all beads in the photometry image tile and per flow. Other functions (such as weighted sums, linear or non-linear functions may be used). This local information may be used for compensating for non-uniformity across the substrate (for example, some tiles may be illuminated with stronger radiation than another tile).

As another example, an additional type of information may include information indicative of the flow base (base used during the flow) and/or the flow position. Such additional information may include a flow base synthetic integer vector and a flow position synthetic integer vector. Any other representation of the fourth additional type of information may be provided.

A U-net of systems, methods, and media of the present disclosure can be, for example, a 6-layer CNN model parallel concatenated to an encoder-decoder. The model may include a number of parameters of about 1 thousand, 5 thousand, 10 thousand, 50 thousand, 100 thousand, 200 thousand, 300 thousand, 400 thousand, 500 thousand, 600 thousand, 700 thousand, 800 thousand, 900 thousand, 1 million, or more than 1 million. Further, the model may be trained using about 1 million, 5 million, 10 million, 15 million, 20 million, 25 million, 30 million, 35 million, 40 million, 45 million, 50 million, 55 million, 60 million, 65 million, 70 million, 75 million, 80 million, 85 million, 90 million, 95 million, 100 million, 150 million, 200 million, 250 million, 300 million, 350 million, 400 million, 450 million, 500 million, 600 million, 700 million, 800 million, 900 million, or 1 billion reads. Reads representing the ground truth may be created by alignment, and reads used in the training may be selected based on a high confidence of alignment. Reads with suspected variance and reads where the information ends before the end of the sequence may be discarded from training.

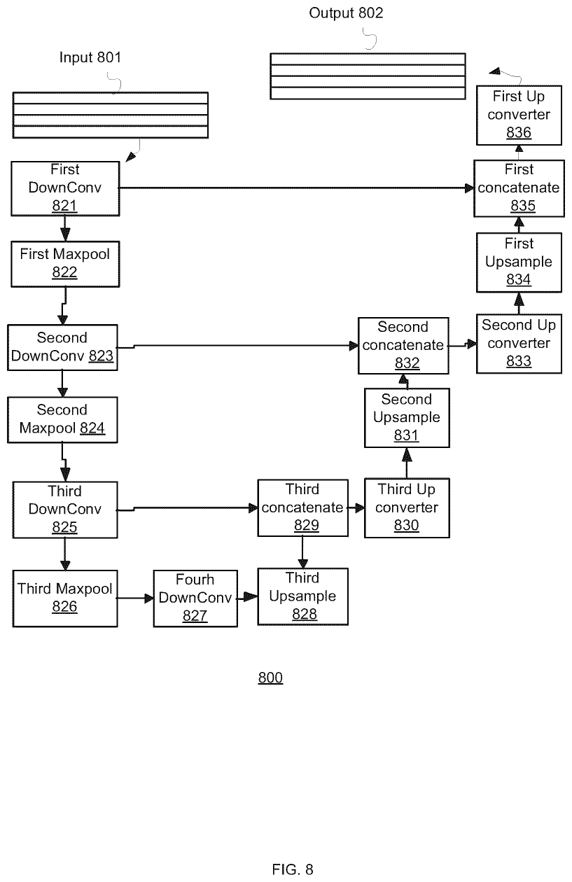

FIG. 8 shows an example of a U-Net 800 that is trained to estimate a genome of a subject of a second genus. In some embodiments, U-Net 800 may be trained and/or applied according to one or more operations of method 100 and/or 200. In some embodiments, U-Net 800 may be provided with input 801, which may include actual human fragment sequencing signals and optionally one or more other additional types of information. In some embodiments, output 802 may include, for example, accurate human fragment sequencing signals.

In some embodiments, as illustrated in FIG. 8, U-Net 800 includes first to fourth down-convolution units (“DownConv”) 821, 823, 825, and 827, first to third maxpool units 822, 824, and 826, first to third upsample units 834, 831, and 828, first to third concatenate units 835, 832, and 829, and first to third up-convolution units 830, 833, and 836.

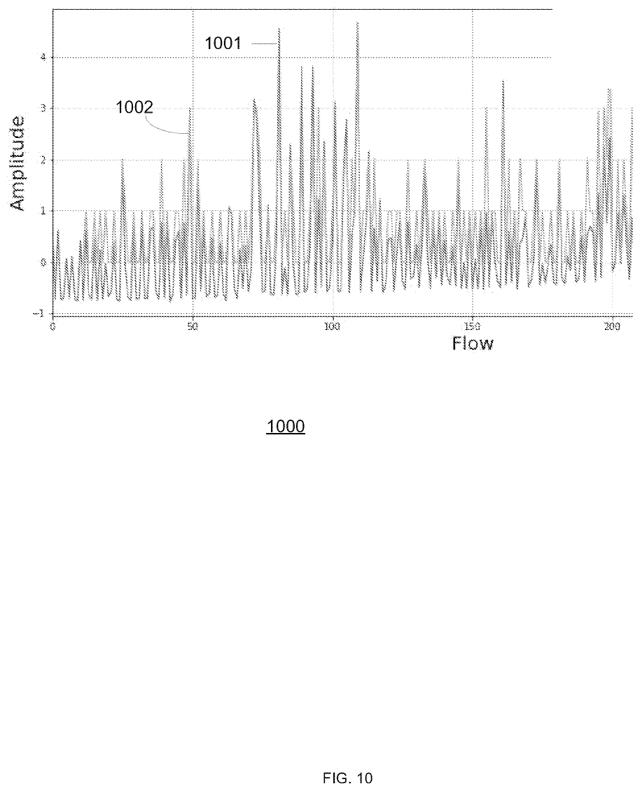

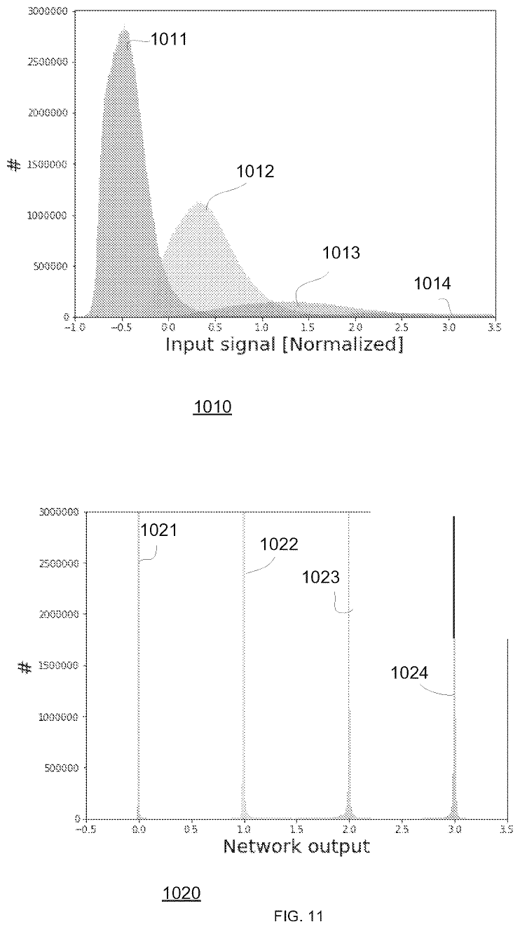

FIG. 10 and FIG. 11 show examples of an input signal that are fed to a neural network and an output generated by the neural network. The input signals comprise actual sequencing signals (e.g., having inaccuracies and noise) that represent a measured number of nucleotides per homopolymer, and the output signal comprises noise-free (or noise-reduced) signals that represent the estimated number of nucleotides per homopolymer.

FIG. 10 shows an example of a graph 1000 that illustrates input signals 1001 and output signals 1002 (e.g., the amplitudes of input and output signals). In some instances, the output signals 1002 correspond to a range of values from about 0, 1, 2, and 3 (i.e., indicating 0, 1, 2, or 3 nucleotides per homopolymer). In some instances, the input signals 1001 correspond to a larger range of values than the output signals 1002.

FIG. 11 illustrates examples of input signal histograms 1010 and output signal histograms 1020. Each input signal histogram is correlated by the neural network (e.g., the trained neural network) to an output signal histogram. That is, each input signal that falls within a respective input signal histogram is mapped by the neural network to a corresponding output signal histogram. The input signal histograms represent sequencing signal values received at a detector. The output signals represent different h-mer values. In some embodiments, each output signal histogram has a value that is approximately an integer (e.g., 0, 1, 2, 3, etc.).

In some embodiments, a first distribution 1011 of input values are mapped by the neural network to a first output distribution 1021 about value zero. That is, input values within the first distribution 1011 are interpreted as corresponding to h-mers of 0 bases (e.g., no incorporation of a nucleotide into a sequencing template nucleic acid). In some embodiments, a second distribution 1012 of input values are mapped by the neural network to a second output distribution 1022 about value one (e.g., input values within the second distribution 1012 are interpreted as corresponding to h-mers of 1 base). In some embodiments, a third distribution 1013 of input values are mapped by the neural network to a third output distribution 1023 about value two (e.g., input values within the third distribution 1013 are interpreted as corresponding to h-mers of 2 bases). In some embodiments, a fourth distribution 1014 of input values are mapped by the neural network to a fourth output distribution 1024 about value three (e.g., input values within the fourth distribution 1014 are interpreted as corresponding to h-mers of 3 bases). It will be understood that additional distributions of input values can similarly be mapped by the neural network to additional distributions of output values. In some instances, one or more of the output distributions may be approximately a delta function.

In some embodiments, a computer system may be used to perform operations of methods of the present disclosure over time and to generate one or more estimates of genomes of one or more organisms.

In some embodiments, at least one of mechanical conditions, inspection conditions, collection conditions, and chemical conditions may change over time, thereby causing one or more models (e.g., machine learning models) that were once accurate to become inaccurate. Accordingly, such models may be replaced, adjusted, or amended over time as needed. For example, the amendment may comprise modifying an initial model that was produced at the initial setup of the computer system (e.g., using one or more features of the initial model for training an updated model). Any method as disclosed herein may be used to generate the initial model.

In some embodiments, the initial model is amended and/or replaced over time. In some instances, the initial model may be amended and/or replaced one or more times. In some instances the initial model may be amended and/or replaced periodically (e.g., each day, each week, each month, each year, etc.). In some embodiments, the model replacement or change occurs in a periodic manner, in response to certain events, after running each estimation, and/or after running multiple (n) estimations. In other cases, the model replacement or change may be triggered upon manual calibration procedures.

The initial model may be amended or replaced, for example, by retraining a trained algorithm (e.g., the trained initial model) using new actual sequencing signals. The new actual sequencing signals may comprise information acquired during one or more completed estimations (e.g., one or more new sets of sequencing data) or information that was not previously processed (e.g., additional information from the initial sequencing data set).

A model replacement may occur (may be initiated) based on an evaluation of a current model. In some instances, such an evaluation may comprise inferring a sample of new actual sequencing signals using the model that was used in a previous estimation. From the sample, a ground truth may be created using an alignment procedure. The inferred results and the new ground truth may be compared, and an error rate or any other reliability or accuracy score may be calculated. If the resulting reliability or accuracy score exceeds a predetermined quality threshold, then the current model may be maintained. If the resulting reliability or accuracy score does not exceed the predetermined quality threshold, then the sample data may be used to train a trained algorithm (e.g., machine learning process) to provide a new model for the new actual sequencing signals.

The retraining of a trained algorithm (e.g., a machine learning process) may comprise training the machine learning process to generate a new model for each set of sequencing data (e.g., de novo) or obtaining a previously used model and running one or more epochs of the previously used model to update the model. The retraining may be executed in various manners, such as applying transfer learning and adjusting only a part of the model (for example, adjusting one or more initial input layers in the model). Such efficient retraining may be needed as training time constraints become critical.

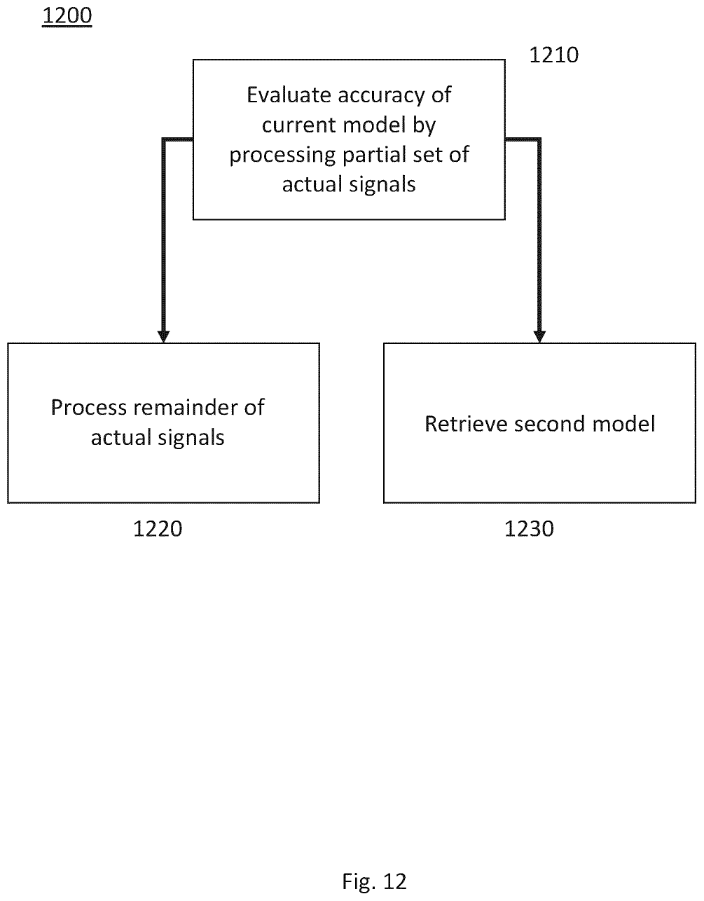

FIG. 12 illustrates an example of a method 1200 for estimating a genome of a genus. The method 1200 may comprise (a) receiving or generating actual sequencing signals that represent a first part of the genome of the genus; (b) applying a current model on at least a portion of the actual sequencing signals to provide partial current results; wherein the current model is generated by a trained algorithm (e.g., machine learning process); (c) evaluating an accuracy of the partial current results; and (d) determining, based on the accuracy of the partial current results, whether to continue using the current model for completing the estimation of the genome (e.g., using the current genome) (as in operation 1210). The accuracy of the partial current results may be evaluated using any of the methods described herein (e.g., processing against ground truth).

In some instances, where method 1200 has determined to continue using the current model, operation 1210 may be followed by completing the estimation of the genome using the current model (as in operation 1220). In some instances, where method 1200 has determined not to continue using the current model, operation 1210 may be followed by obtaining a second model having sufficient estimation accuracy, and estimating the genome (e.g., of the second genus) using the second model (as in operation 1230). In some instances, the current model may be retrained or amended and operation 1210 repeated until it is determined that the evaluated model has sufficient accuracy.

In some embodiments, the current model is generated based on information corresponding to a reference genome that is smaller than (e.g., significantly smaller than) the genome of the genus. For example, as described in any of the methods disclosed herein, a first genome (reference genome) may be used that is shorter than the second genome. In some embodiments, a first genome may be substantially similar in size to the second genome.

The estimation may be performed by a computer system. In some embodiments, at least one model that was used by the computer system prior to using the current model is generated based on information corresponding to a reference genome that is smaller (e.g., significantly smaller) than the genome of the genus. The at least one model may be the initial model or any other model. In some embodiments, the method 1200 may comprising executing a plurality of iterations of the set of operations 1210, 1220, and 1230.

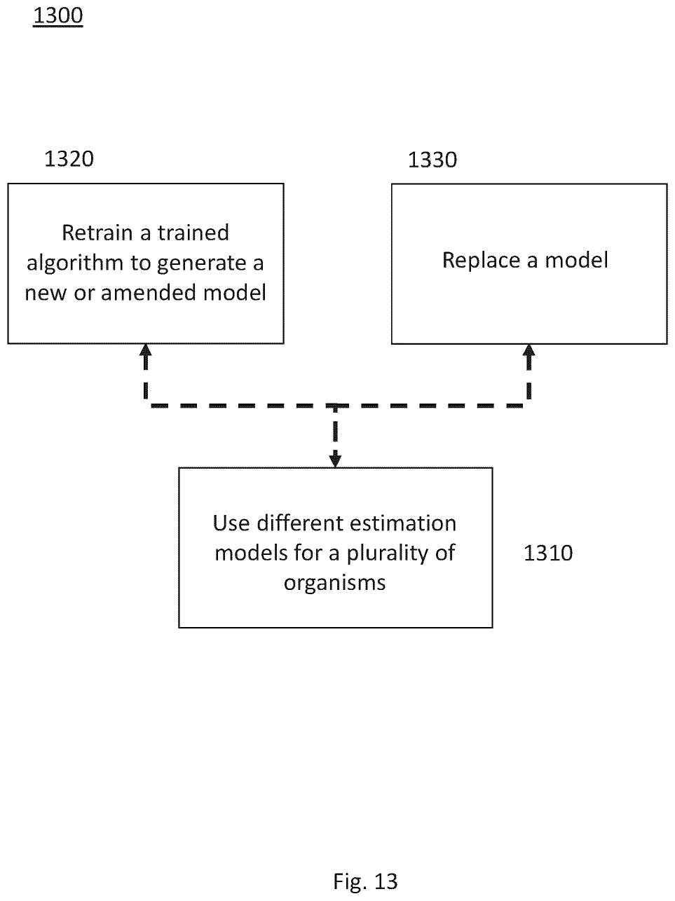

FIG. 13 illustrates an example of a method 1300 for estimating genomes of a plurality of organisms of a genus.

The method 1300 may comprise performing a plurality of different estimation processes for estimating the genomes of the plurality of organisms (as in operation 1310). In some embodiments, performing the plurality of estimation processes comprises using a plurality of different estimation models. In some embodiments, at least one of the plurality of different models is generated by retraining a trained algorithm (e.g., machine learning process) to provide a new and/or amended model (as in operation 1320). In some embodiments, the retraining is performed based, at least in part, on information corresponding to a reference genome that is smaller (e.g., significantly smaller) than the genome of the genus (e.g., a second genome). In some embodiments, the at least one of the plurality of different models is generated based on information corresponding to a reference genome that is smaller (e.g., significantly smaller) than the genome of the genus. In some embodiments, the method 1300 may comprise replacing a model of the plurality of different models by a second model during each of a plurality of predefined durations of time (as in operation 1330). In some embodiments, the method 1300 may comprise replacing a model of the plurality of different models by a second model during each of a plurality of predefined numbers of estimation processes. In some embodiments, the method 1300 may comprise replacing a model of the plurality of different models by a second model based on an evaluation of an accuracy of the model.

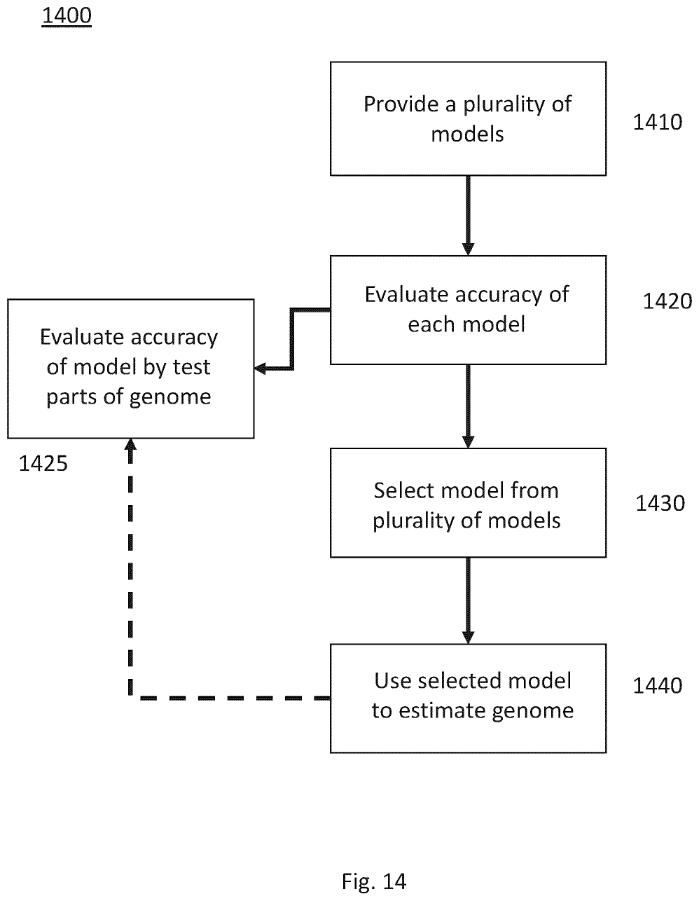

FIG. 14 illustrates an example of a method 1400 for estimating a genome of a genus. The method 1400 may comprise estimating the genome of the genus. The estimating may include providing a plurality of models (as in operation 1410); selecting a model to be used during the estimation process, out of a plurality of models (as in operation 1430); and using the selected model to estimate the genome (as in operation 1440). In some embodiments, the selecting may be performed based at least in part on an estimate regarding an accuracy of the estimation corresponding to the plurality of models (e.g., as in operation 1420).

In some embodiments, the estimate may be performed based on tests made on regions of the genome (e.g., as in operation 1425). The accuracy of the model may be evaluated using any of the methods described herein (e.g., processing against ground truth). For example, the accuracy of the model may be evaluated using a statistical measure of error, such as an R-squared value, a mean squared error (MSE), a root mean squared error (RMSE), a sum of squares error (SSE), a mean absolute error (MAE), a mean absolute percentage error (MAPE), etc. (e.g., where a lower measure of error indicates a higher accuracy of the model). In some instances, each model may be tested on a single portion of the genome, or multiple portions of the genome. In some instances, a model may be evaluated by testing a reference genome. In some instances, a model may be evaluated by testing another genome. For example, one or more portions of the genome may be compared to a reference genome or another genome to evaluate the accuracy of the model.

In some embodiments, the method 1400 may comprise selecting one or more models from a plurality of models, and using the selected one or more models to estimate the genome. For example, the same genome may be estimated based on a plurality of model to generate a plurality of estimates. The plurality of estimates may be further processed to, for example, generate a consolidated estimate. The plurality of estimates may be used to evaluate the selected models (as in operation 1425), such as to determine, whether one or more of such selected models have to be retrained and/or amended. For example, an estimate that diverges substantially from a remainder of the estimates may be indicative of an inaccurate model.

Provided herein is a method for estimation of a genome of a genus. The method may comprise performing a plurality of different estimation processes for estimating the genomes of a plurality of multiple organisms; wherein an estimation process of the plurality of different estimation processes comprises selecting a model from among a plurality of different models to be used during the estimation process.

In some embodiments, the selecting is based on an estimate regarding an accuracy of the estimation corresponding to the plurality of models. In some embodiments, the estimating is based on tests made on regions of the genome.

In some embodiments, the estimating is performed by a computer system.

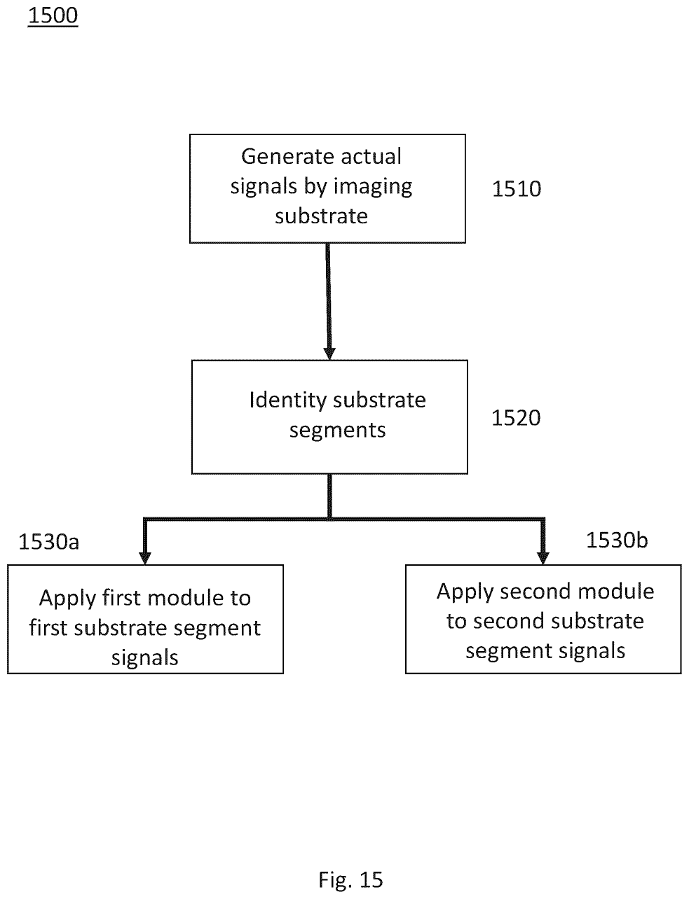

FIG. 15 illustrates an example of a method 1500 for estimating a genome of a genus.

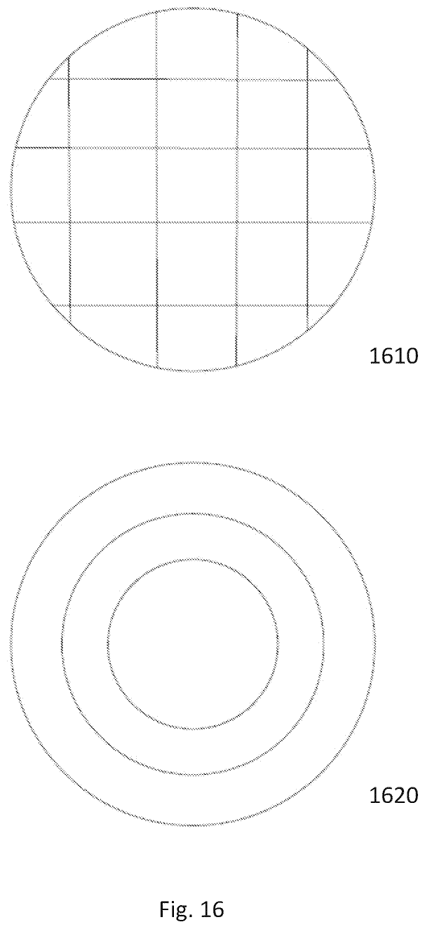

The method 1500 may comprise receiving or generating actual sequencing signals that represent at least a part of the genome of the genus. The actual sequencing signals may be generated by imaging a substrate that may include a plurality of substrate segments (as in operation 1510). FIG. 16 shows two examples of substrate (e.g., wafers) and segments thereof—wafer 1610 with segments thereof (e.g., arranged in a grid-like pattern), and wafer 1620 with segments thereof (e.g., arranged in a concentric circle pattern). It will be appreciated that the substrate may be segmented in any arrangement, pattern, or configuration into any number of segments.

The method 1500 may comprise identifying different substrate segments (as in operation 1520). In some cases, the different substrate segments may be identified prior to imaging, during imaging, or subsequent to imaging. For example, prior to imaging, the substrate may be segmented into different segments which may or may not be demarcated. In another example, subsequent to imaging, the different substrate segments may be identified from one or more images from the imaging. Any number of substrate segments may be identified.

In some instances, the method 1500 may comprise estimating the genome of the genus by applying a first module to signals (e.g., from among the actual sequencing signals) associated with a first substrate segment of the plurality of substrate segments and applying a second module that differs from the first module on signals (e.g., from among the actual sequencing signals) associated with a second substrate segment of the plurality of substrate segments. A different module may be applied to each of the different substrate segments. A module may be applied to multiple different substrate segments. In some cases, a set of identified substrate segments may be grouped into a plurality of groups, and a different module may be applied to each group such that the same module is applied to each member of a group. A module may comprise a model as described elsewhere herein.

In some embodiments, the plurality of substrate segments are determined based on expected or actual differences between an illumination of the plurality of substrate segments. In some embodiments, the plurality of substrate segments are determined based on expected or actual differences between a collection or measurement of radiation from the plurality of substrate segments. In some embodiments, the plurality of substrate segments are determined based on expected or actual distribution of chemical materials over the plurality of substrate segments.

In some embodiments, the plurality of substrate segments are determined based on expected or actual distribution of samples or sample sources over the plurality of substrate segments. For example, such samples (e.g., comprising a plurality of beads, each bead comprising a clonal population of amplified products) may be immobilized at different substrate segments.

In some embodiments, the plurality of substrate segments comprise a same shape and/or size. In some embodiments, at least two of the plurality of substrate segments differ by at least one of shape and size.

Provided herein is a method for estimating a genome of a genus. The method may comprise receiving or generating actual sequencing signals that represent at least a part of the genome of the genus; wherein the actual sequencing signals belong to at least one image of at least one part of a substrate that is linked to multiple DNA beads.

In some embodiments, the method may further comprise estimating the genome of the genus by applying at least one model to the actual sequencing signals.

Generating Sequencing Data Using Flow Sequencing Methods

Sequencing data can be generated using a flow sequencing method that includes extending a primer hybridized to a template polynucleotide molecule according to a pre-determined flow cycle or flow order where, in any given flow position, a type of nucleotide base is accessible to the extending primer. More commonly, a single type of nucleotide base is used in any given sequencing flow, although in some variations, two or three different types of nucleotide bases may be used, which allows for a faster primer extension but may provide less sequencing data about the sequence region covered. At least some of the nucleotides of the particular base type can include a label, which upon incorporation of the labeled nucleotides into the extending primer renders a detectable signal. The resulting sequence by which such nucleotides are incorporated into the extended primer should be the reverse complement of the sequence of the template polynucleotide molecule. For example, sequencing data may be generated using a flow sequencing method that includes extending a primer using labeled nucleotides, and detecting the presence or absence of a labeled nucleotide incorporated into the extending primer. Flow sequencing methods may also be referred to as “natural sequencing-by-synthesis,” or “non-terminated sequencing-by-synthesis” methods. Exemplary methods are described in U.S. Pat. No. 8,772,473; published International application WO 2021/007495; published International application WO 2020/0227143; and published International application WO 2020/227137; each of which is incorporated herein by reference in its entirety. While the following description is provided in reference to flow sequencing methods, it is understood that other sequencing methods may be used to sequence all or a portion of the sequenced region.

Flow sequencing includes the use of nucleotides to extend the primer hybridized to the polynucleotide. Nucleotides of a given base type (e.g., A, C, G, T, U, etc.) can be mixed with hybridized templates to extend the primer if a complementary base is present in the template strand. The nucleotides may be, for example, non-terminating nucleotides. When the nucleotides are non-terminating, more than one consecutive base can be incorporated into the extending primer strand if more than one consecutive complementary base is present in the template strand. The non-terminating nucleotides contrast with nucleotides having 3′ reversible terminators, wherein a blocking group is generally removed before a successive nucleotide is attached. If no complementary base is present in the template strand, primer extension ceases until a nucleotide that is complementary to the next base in the template strand is introduced. At least a portion of the nucleotides can be labeled so that incorporation can be detected. Most commonly, only a single nucleotide type is introduced at a time (i.e., discretely added), although two or three different types of nucleotides may be simultaneously introduced in certain embodiments. This methodology can be contrasted with sequencing methods that use a reversible terminator, wherein primer extension is stopped after extension of every single base before the terminator is reversed to allow incorporation of the next succeeding base.

The nucleotides can be introduced at a determined order during the course of primer extension, which may optionally be further divided into cycles. Nucleotides are added stepwise, which allows incorporation of the added nucleotide to the end of the sequencing primer of a complementary base in the template strand is present. The cycles may have the same order of nucleotides and number of different base types or a different order of nucleotides and/or a different number of different base types. Solely by way of example, the order of a first cycle may be A-T-G-C and the order of a second cycle may be A-T-C-G. In some instances, the order of any cycle may be any permutation of the nucleotides A, G, C, and T (or U). Between the introductions of different nucleotides, unincorporated nucleotides may be removed, for example by washing the sequencing platform with a wash fluid.

A polymerase can be used to extend a sequencing primer by incorporating one or more nucleotides at the end of the primer in a template-dependent manner. In some embodiments, the polymerase is a DNA polymerase. The polymerase may be a naturally occurring polymerase or a synthetic (e.g., mutant) polymerase. The polymerase can be added at an initial step of primer extension, although supplemental polymerase may optionally be added during sequencing, for example with the stepwise addition of nucleotides or after a number of flow cycles. Exemplary polymerases include a DNA polymerase, an RNA polymerase, a thermostable polymerase, a wild-type polymerase, a modified polymerase, Bst DNA polymerase, Bst 2.0 DNA polymerase Bst 3.0 DNA polymerase, Bsu DNA polymerase, E. coli DNA polymerase I, T7 DNA polymerase, bacteriophage T4 DNA polymerase 129 (phi29) DNA polymerase, Taq polymerase, Tth polymerase, Tli polymerase, Pfu polymerase, and SeqAmp DNA polymerase.

The introduced nucleotides can include labeled nucleotides when determining the sequence of the template strand, and the presence or absence of an incorporated labeled nucleic acid can be detected to determine a sequence. The label may be, for example, an optically active label (e.g., a fluorescent label) or a radioactive label, and a signal emitted by or altered by the label can be detected using a detector. The presence or absence of a labeled nucleotide incorporated into a primer hybridized to a template polynucleotide can be detected, which allows for the determination of the sequence (for example, by generating a flowgram). In some embodiments, the labeled nucleotides are labeled with a fluorescent, luminescent, or other light-emitting moiety. In some embodiments, the label is attached to the nucleotide via a linker. In some embodiments, the linker is cleavable, e.g., through a photochemical or chemical cleavage reaction. For example, the label may be cleaved after detection and before incorporation of the successive nucleotide(s). In some embodiments, the label (or linker) is attached to the nucleotide base, or to another site on the nucleotide that does not interfere with elongation of the nascent strand of DNA. In some embodiments, the linker comprises a disulfide or PEG-containing moiety.

In some embodiment, the nucleotides introduced include only unlabeled nucleotides, and in some embodiments the nucleotides include a mixture of labeled and unlabeled nucleotides. For example, in some embodiments, the portion of labeled nucleotides compared to total nucleotides is about 90% or less, about 80% or less, about 70% or less, about 60% or less, about 50% or less, about 40% or less, about 30% or less, about 20% or less, about 10% or less, about 5% or less, about 4% or less, about 3% or less, about 2.5% or less, about 2% or less, about 1.5% or less, about 1% or less, about 0.5% or less, about 0.25% or less, about 0.1% or less, about 0.05% or less, about 0.025% or less, or about 0.01% or less. In some embodiments, the portion of labeled nucleotides compared to total nucleotides is about 100%, about 95% or more, about 90% or more, about 80% or more about 70% or more, about 60% or more, about 50% or more, about 40% or more, about 30% or more, about 20% or more, about 10% or more, about 5% or more, about 4% or more, about 3% or more, about 2.5% or more, about 2% or more, about 1.5% or more, about 1% or more, about 0.5% or more, about 0.25% or more, about 0.1% or more, about 0.05% or more, about 0.025% or more, or about 0.01% or more. In some embodiments, the portion of labeled nucleotides compared to total nucleotides is about 0.01% to about 100%, such as about 0.01% to about 0.025%, about 0.025% to about 0.05%, about 0.05% to about 0.1%, about 0.1% to about 0.25%, about 0.25% to about 0.5%, about 0.5% to about 1%, about 1% to about 1.5%, about 1.5% to about 2%, about 2% to about 2.5%, about 2.5% to about 3%, about 3% to about 4%, about 4% to about 5%, about 5% to about 10%, about 10% to about 20%, about 20% to about 30%, about 30% to about 40%, about 40% to about 50%, about 50% to about 60%, about 60% to about 70%, about 70% to about 80%, about 80% to about 90%, about 90% to less than 100%, or about 90% to about 100%.

The sequencing data can be generated by sequencing the test nucleic acid molecule using non-terminating nucleotides provided in separate nucleotide flows according to a flow-cycle order. The sequencing data can include flow signals at flow positions that each corresponds to a flow of a particular nucleotide. Using this uniquely structured data set, the nucleic acid molecule (or molecules) can be analyzed in “flowspace” rather than “basespace” (also referred to as “nucleotide space” or “sequence space”). The flowspace data depend on additional information related to the flow-cycle order, which is not included in basespace data. See, for example, published International application WO 2020/227137, which is incorporated herein by reference in its entirety.

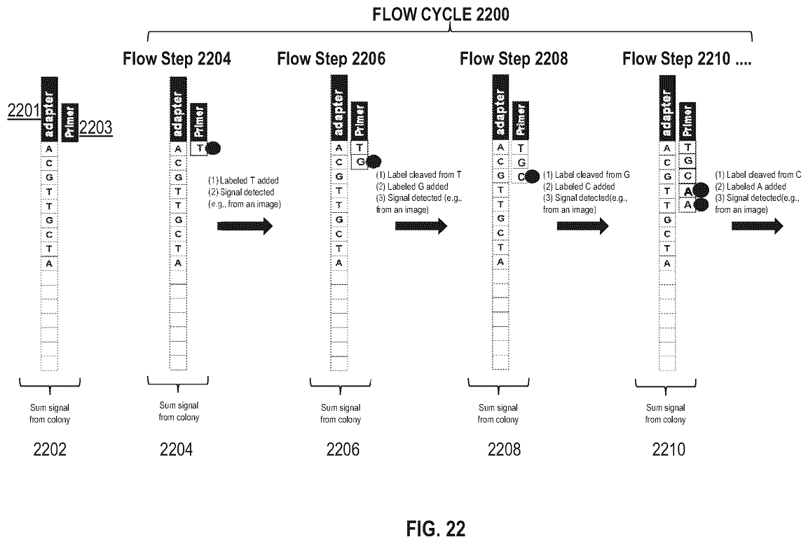

FIG. 22 illustrates an exemplary flow sequencing method that can be used to generate the sequencing data described herein. In some embodiments, polynucleotides may be bound to a surface (e.g., the surface of a bead attached to a substrate), as described in detail herein. The polynucleotides can include a nucleic acid sequence of interest (also referred to as a “template sequence”) and can further include a sequencing adapter sequence. The nucleic acid sequence of interest can be a nucleic acid molecule from or derived from a sample of a subject.

As illustrated in FIG. 22, the nucleic acid sequence of interest includes an adaptor sequence 2201 followed by the nucleic acid sequence of interest (“ACGTTGCTA . . . ”). In some instances, the adapter sequence 2201 can include a sequencing primer hybridization site. At step 2202, in some instances, a sequencing primer 2203 is hybridized to the adapter sequence 2201 of the polynucleotide at the sequencing primer hybridization site.

The sequencing primer is then extended in a series of flow cycles. In a flow cycle, the hybrid (i.e., the polynucleotide adapter hybridized to the sequencing primer) is combined with nucleotides (e.g., at least partially labeled nucleotides) and one or more signals indicating nucleotide incorporation into the sequencing primer may be detected. In the depicted example, the flow cycle 2200 includes four flow steps 2204, 2206, 2208, and 2210. In a given flow step, a single type of nucleobase is combined with the hybrid according to the flow-cycle order T-G-C-A. As shown in FIG. 22, in flow step 2204, labeled T nucleotides are combined with the hybrid; in flow step 2206, labeled G nucleotides are combined with the hybrid; in flow step 2208, labeled C nucleotides are combined with the hybrid; in flow step 2210, labeled A nucleotides are combined with the hybrid.

At flow step 2204, labeled T nucleotides are combined with the hybrid. Since the T base is complementary to the A base in the template polynucleotide, it is incorporated into the extending primer to form the hybrid as shown in step 2204. Further, a signal indicative of the incorporation of labeled T nucleotide into the sequencing primer can be detected. The signal may be detected, for example, by imaging the surface the polynucleotides are deposited on and analyzing the resulting image(s). In some embodiments, the sequencing platform may be washed with a wash buffer to remove unincorporated nucleotides prior to signal detection. In some embodiments, the detection of the signal is based on image processing techniques described herein.

At flow step 2206, the label may be removed from the T nucleotide (e.g., by cleaving the label from the nucleotide). The sequencing method can then be continued with the next base in the flow order, G in the example illustrated in FIG. 22. At step 2206, labeled G nucleotides are combined with the hybrid. Since the G base is complementary to the C base in the template polynucleotide, it is incorporated to form the hybrid in flow step 2206. Further, a signal indicating the incorporation of the labeled G nucleotide can be detected.

At step 2208, the label may be removed from the G nucleotide (e.g., by cleaving the label from the nucleotide). The sequencing method can then be continued with the next base in the flow order, C. At step 2208, labeled C nucleotides are combined with the hybrid. Since the C base is complementary to the G base in the template polynucleotide, it is incorporated into the extending primer to form the hybrid in flow step 2208. Further, a signal indicating the incorporation of the labeled C nucleotide into the sequencing primer can be detected.

At step 2210, the label may be removed from the C nucleotide (e.g., by cleaving the label from the nucleotide). The sequencing method can then be continued with the next base in the flow order, A. At step 2210, labeled A nucleotides are combined with the hybrid. Since the A base is complementary to the T base in the template polynucleotide, it is incorporated into the extending primer to form the hybrid in flow step 2210. Further, a signal indicating the incorporation of the labeled A nucleotide into the sequencing primer can be detected.

In step 2210, because the template sequence includes two consecutive T bases, two A nucleotides are incorporated into the extending sequencing primer (e.g., an h-mer of 2). Thus, the detected signal intensity indicating the incorporation of two A nucleotides may be greater than the signal intensity indicating the incorporation of one nucleotide. Likewise, detected signal intensity indicating incorporation of three nucleotides may be greater that the signal intensity indicating the incorporation of two nucleotides (and similarly for other detected signal intensities indicating incorporation of more nucleotides—e.g., 4, 5, 6, 7, etc. nucleotides).

While each flow step in the exemplary flow sequencing method in FIG. 22 results in incorporation of one or more nucleotides (and thus a detected signal indicating such incorporation), it should be appreciated that not all flow steps result in incorporation of nucleotides. In some flow steps, no nucleotide base may be incorporated (for example, in the absence of a complementary base in the template polynucleotide). For example, if C nucleotides are combined with a hybrid having a C base, no incorporation would occur and thus no signal indicative of an incorporation would be detected. Further, as shown in step 2210, two nucleotides or more than two nucleotides may be incorporated into the sequencing primer for larger homopolymer lengths in the nucleic acid sequence of interest.

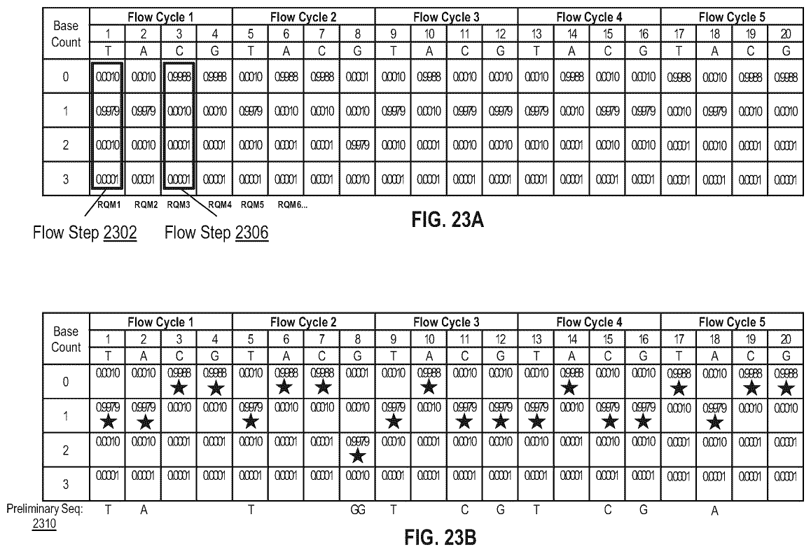

FIG. 23A illustrates an exemplary summary of detected signals after five exemplary flow cycles are performed, in accordance with some embodiments. Solely by way of example, a primer extended using a repeating flow-cycle order of T-A-C-G may result in a sequencing data flowgram set shown in FIG. 23A. Each column in FIG. 23A corresponds to a flow step and the values in each column collectively represent the detected signal intensity in the corresponding flow step, as described below. In some instances, the data in FIG. 23A is exemplary of flowspace data.

In each flow step, the flow signal can be determined from an analog signal that is detected during the sequencing process, such as a fluorescent signal of the one or more bases incorporated into the sequencing primer during sequencing. Although an integer number of zero or more bases are incorporated at any given flow position, a given analog signal many not perfectly match with the analog signal. Therefore, in some embodiments, for a given flow step (e.g., flow step 2302), the detected signal intensity can be expressed in probabilistic terms. Specifically, the detected signal intensity can be expressed in four likelihood values corresponding to 0 bases, 1 base, 2 bases, and 3 bases, respectively.

In the depicted example, for flow step 2302, the detected signal intensity is expressed by a first likelihood value of 0.001 for 0 base, a second likelihood value of 0.9979 for 1 base, a third likelihood value of 0.001 for 3 bases, and a fourth likelihood value of 0.0001 for 4 bases. This can be interpreted to indicate that there is a high statistical likelihood that one nucleotide base has been incorporated. In the depicted example, the incorporation is a T since the flow step introduced labeled T nucleotides, which means there is an A in the template.

On the other hand, in flow step 2306, the detected signal intensity is expressed by a first likelihood value of 0.9988 for 0 base, a second likelihood value of 0.001 for 1 base, a third likelihood value of 0.001 for 3 bases, and a fourth likelihood value of 0.0001 for 4 bases. This can be interpreted to indicate that there is a high likelihood that no nucleotide base has been incorporated. Indeed, in the depicted example, no C has been incorporated.

Accordingly, the flowgram set in FIG. 23A is formatted as a sparse matrix, with a flow signal represented by a plurality of likelihood values indicating a plurality of likelihoods for a plurality of base homopolymer length counts (e.g., 0 base count, 1 base count, 2 base counts, and 3 base counts) at each flow position.

The homopolymer length likelihood may vary, for example, based on the noise or other artifacts present during detection of the analog signal during sequencing. In some embodiments, if the homopolymer length likelihood statistical parameter or likelihood is below a predetermined threshold, the parameter may be set to a predetermined non-zero value that is substantially zero (i.e., some very small value or negligible value) to aid the downstream statistical analysis further discussed herein, wherein a true zero value may give rise to a computational error or insufficiently differentiate between levels of unlikelihood, e.g., very unlikely (0.0001) and inconceivable (0).

With reference to FIG. 23B, a preliminary sequence can be determined based on the flowgram in FIG. 23A. For example, the most likely sequence can be determined by selecting the base count with the highest likelihood at each flow position, as shown by the stars in FIG. 23B. Thus, the preliminary sequence 2310 can be determined as: TATGGTCGTCGA.

From the preliminary sequence (e.g., preliminary sequence 2310), the reverse complement (i.e., the template strand or the nucleic acid sequence of interest) can be readily determined. Further, the likelihood of this sequencing data set, given the TATGGTCGTCGA sequence (or the reverse complement), can be determined as the product of the selected likelihood at each flow position.

The signal for any flow position in the sequencing data is flow-order-dependent in that the flow order used to sequence the polynucleotide at any base position can affect the flow signal at that position. Random fragmentation of nucleic acid molecules (either in vivo fragmentation, such as cell-free DNA, or in vitro fragmentation, such as by sonication or enzymatic digestion) that overlap at the same locus results in multiple different sequencing start sites (relative to the locus) for the nucleic acid molecules.

Sequencing data, such as a flowgram, is based on the detection of a signal detected from an incorporated nucleotide and the order of nucleotide introduction. Take, for example, the flowing template sequences: CTG and CAG, and a repeating flow cycle of T-A-C-G (that is, sequential addition of T, A, C, and G nucleotides, each of which would be incorporated into the primer only if a complementary base is present in the template polynucleotide). A resulting exemplary flowgram is shown in Table 1, where 1 indicates incorporation of an introduced nucleotide and 0 indicates no incorporation of an introduced nucleotide. The flowgram can be used to determine the sequence of the template strand.

TABLE 1

| | Cycle 1 | | | | Cycle 2 | | | |
|---|---|---|---|---|---|---|---|---|
| Sequence | T | A | C | G | T | A | C | G |
| CTG | 0 | 0 | 0 | 1 | 0 | 1 | 1 | 0 |
| CAG | 0 | 0 | 0 | 1 | 1 | 0 | 1 | 0 |
| CCG | 0 | 0 | 0 | 2 | 0 | 0 | 1 | 0 |

The flowgram can be used to quantitatively determine a number of incorporated nucleotides from each stepwise introduction (e.g., for each nucleotide in a cycle). For example, a sequence of CCG would first incorporate two G bases, and any signal emitted by the labeled two bases would have a greater intensity as compared with the incorporation of a single base. This is shown in Table 1 (e.g., the 2 value in the third row). The flowgram of Table 1 indicates the presence or absence of each indicated base, but flowgrams can also provide additional information including the number of bases incorporated at the given step.

Prior to generating the sequencing data, the polynucleotide is hybridized at a hybridization site to a sequencing primer to generate a hybridized template. The polynucleotide may be ligated to an adapter during sequencing library preparation, such as during the attachment of one or more barcode regions. The adapter can include a hybridization sequence that hybridizes to the sequencing primer. For example, the hybridization sequence of the adapter may be a uniform sequence across a plurality of different polynucleotides, and the sequencing primer may be a uniform sequencing primer. This allows for multiplexed sequencing of different polynucleotides in a sequencing library.

The polynucleotide may be attached to a surface (such as a solid support) for sequencing. The polynucleotides may be amplified (for example, by bridge amplification or other amplification techniques) to generate polynucleotide sequencing colonies. The amplified polynucleotides within the cluster are substantially identical or complementary (some errors may be introduced during the amplification process such that a portion of the polynucleotides may not necessarily be identical to the original polynucleotide). Colony formation allows for signal amplification so that the detector can accurately detect incorporation of labeled nucleotides for each colony. In some cases, the colony is formed on a bead using emulsion PCR and the beads are distributed over a sequencing surface. Examples for systems and methods for sequencing can be found in U.S. Pat. No. 10,344,328 and International patent application WO 2020/227143, each of which is incorporated herein by reference in its entirety.

The primer hybridized to the polynucleotide is extended through the nucleic acid molecule using the separate nucleotide flows according to the flow order (which may be cyclical according to a flow-cycle order), and incorporation of a nucleotide can be detected as described above, thereby generating the sequencing data set (via a flowgram) for the nucleic acid molecule.

Alignment (or mapping) of determined sequences to candidate sequences (such as candidate haplotype sequences) in base space is computationally expensive and is currently the most computationally intensive step in, for example, the Genome Analysis Tool Kit (GATK) HaplotypeCaller. Within HaplotypeCaller, PairHMM aligns each sequencing read to each haplotype, and uses base qualities as an estimate of the error to determine the likelihood of the haplotypes given the sequencing read. However, the structure of the data set used with the methods described herein retains error mode likelihoods, which makes variant calling more computationally efficient. For example, a given genotype likelihood may be determined simply as the product of likelihoods in each flow position that aligns with the sequence having the genotype. The flowspace determined likelihood can replace the PairHMNI module of the HaplotypeCaller, thus enabling more computationally efficient variant calling.

Primer extension using flow sequencing allows for long-range sequencing on the order of hundreds or even thousands of bases in length. The number of flow steps or cycles can be increased or decreased to obtain the desired sequencing length. Extension of the primer can include one or more flow steps for stepwise extension of the primer using nucleotides having one or more different base types. In some embodiments, extension of the primer includes between 1 and about 1000 flow steps, such as between 1 and about 10 flow steps, between about 10 and about 20 flow steps, between about 20 and about 50 flow steps, between about 50 and about 100 flow steps, between about 100 and about 250 flow steps, between about 250 and about 500 flow steps, or between about 500 and about 1000 flow steps. The flow steps may be segmented into identical or different flow cycles. The number of bases incorporated into the primer depends on the sequence of the sequenced region, and the flow order used to extend the primer. In some embodiments, the sequenced region is about 1 base to about 4000 bases in length, such as about 1 base to about 10 bases in length, about 10 bases to about 20 bases in length, about 20 bases to about 50 bases in length, about 50 bases to about 100 bases in length, about 100 bases to about 250 bases in length, about 250 bases to about 500 bases in length, about 500 bases to about 1000 bases in length, about 1000 bases to about 2000 bases in length, or about 2000 bases to about 4000 bases in length.

The polynucleotides used in the methods described herein may be obtained from any suitable biological source, for example a tissue sample, a blood sample, a plasma sample, a saliva sample, a fecal sample, or a urine sample. The polynucleotides may be DNA or RNA polynucleotides. In some embodiments, RNA polynucleotides are reverse transcribed into DNA polynucleotides prior to hybridizing the polynucleotide to the sequencing primer. In some embodiments, the polynucleotide is a cell-free DNA (cfDNA), such as a circulating tumor DNA (ctDNA) or a fetal cell-free DNA. The nucleic acid molecules may be randomly fragmented, for example in vivo (e.g., as in cfDNA) or in vitro (for example, by sonication or enzymatic fragmentation).

Libraries of the polynucleotides may be prepared through known methods. In some embodiments, the polynucleotides may be ligated to an adapter sequence. The adapter sequence may include a hybridization sequence that hybridized to the primer extended during the generated of the coupled sequencing read pair.

In some embodiments, the sequencing data is obtained without amplifying the nucleic acid molecules prior to establishing sequencing colonies (also referred to as sequencing clusters). Methods for generating sequencing colonies include bridge amplification or emulsion PCR. Methods that rely on shotgun sequencing and calling a consensus sequence generally label nucleic acid molecules using unique molecular identifiers (UMIs) and amplify the nucleic acid molecules to generate numerous copies of the same nucleic acid molecules that are independently sequenced. The amplified nucleic acid molecules can then be attached to a surface and bridge amplified to generate sequencing clusters that are independently sequenced. The UMIs can then be used to associate the independently sequenced nucleic acid molecules. However, the amplification process can introduce errors into the nucleic acid molecules, for example due to the limited fidelity of the DNA polymerase. In some embodiments, the nucleic acid molecules are not amplified prior to amplification to generate colonies for obtaining sequencing data. In some embodiments, the nucleic acid sequencing data is obtained without the use of unique molecular identifiers (UMIs).

Exemplary Techniques for Improving Sequencing Read Quality

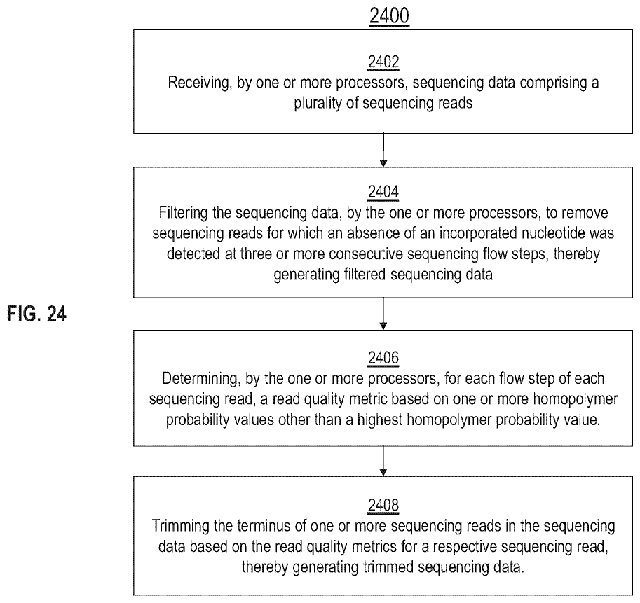

FIG. 24 illustrates an exemplary method 2400 for increasing sequencing read quality, in accordance with some embodiments. In some embodiments, process 2400 is performed, for example, using one or more electronic devices implementing a software platform. In some examples, process 2400 is performed using a client-server system, and the blocks of process 2400 are divided up in any manner between the server and client device(s). In other examples, process 2400 is performed using only a client device or only multiple client devices. In process 2400, some blocks are, optionally, combined, the order of some blocks is, optionally, changed, and some blocks are, optionally, omitted. In some examples, additional steps may be performed in combination with the process 2400. Accordingly, the operations as illustrated (and described in greater detail below) are exemplary by nature and, as such, should not be viewed as limiting.

At block 2402, an exemplary system (e.g., one or more electronic devices) receives, by one or more processors, sequencing data comprising a plurality of sequencing reads. Each sequencing read of the plurality of sequencing reads can be generated according to a flow sequencing method. As discussed above with reference to FIG. 22, each sequencing read can be generated by extending a sequencing primer (e.g., primer 2203) through a region of interest in a target nucleic acid molecule using a plurality of sequencing flow steps (e.g., flow steps 2204, 2206, 2208, 2210). Each sequencing flow step can involve combining a hybrid, which comprises the sequencing primer and a nucleic acid molecule comprising the region of interest, with nucleotides, as shown in each of flow steps 2204, 2206, 2208, and 2210. At least a portion of the nucleotides are labeled (e.g., T in flow step 2204). At each flow step, the presence or absence of an incorporated nucleotide can be detected, and a sequencing read can be generated based on the signals detected over the flow steps, as described with reference to FIGS. 2A-2B. In some embodiments, the nucleotides are non-terminating nucleotides.

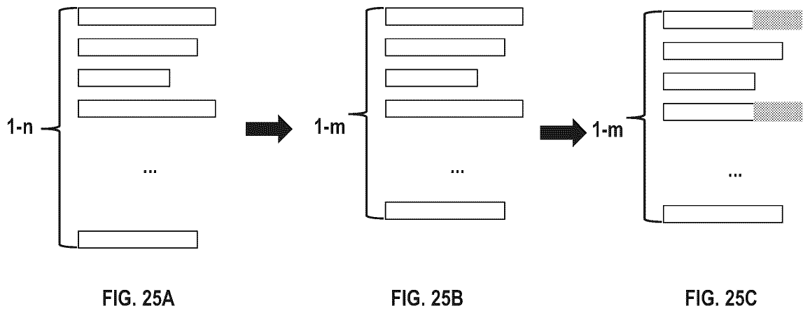

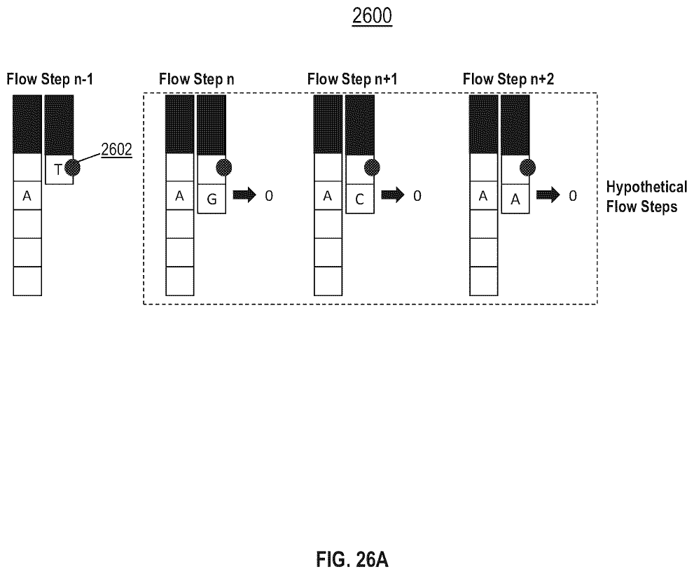

FIG. 25A illustrates an exemplary plurality of sequencing reads that can be received at block 2402 of FIG. 24. In FIG. 25A, the system receives n number of sequencing reads. Each sequencing read is obtained from a flow sequencing method. In some embodiments, the sequencing reads are generated by performing one flow sequencing method on a plurality of sequencing colonies attached to the same surface, where each sequencing read corresponds to a sequencing colony. In some embodiments, the sequencing reads are generated by performing multiple flow sequencing methods. The quality of the plurality of sequencing reads can be improved in blocks 2404-2408, as described below.

At block 2404, the system filters the sequencing data, by the one or more processors, to remove sequencing reads for which an absence of an incorporated nucleotide was detected at three or more consecutive sequencing flow steps, thereby generating filtered sequencing data. Specifically, the system can examine each sequencing read of the plurality of sequencing reads one by one to determine if each sequencing read needs to be filtered (i.e., excluded). For each sequencing read, the system determines if the sequencing read indicates an absence of an incorporated nucleotide at three or more consecutive sequencing flow steps, for example, if the sequencing read indicates three consecutive sequencing flow steps yielding no signals (“000”), four consecutive sequencing flow steps yielding no signals (“0000”), five consecutive sequencing flow steps yielding no signals (“00000”), and so on. If this is so, the sequencing read is excluded from the plurality of sequencing reads. With reference to FIG. 25B, the system can examine each of the sequencing reads 1−n and exclude any sequencing read indicating an absence of an incorporated nucleotide at three or more consecutive sequencing flow steps, thus obtaining sequencing reads 1−m (where m<n).

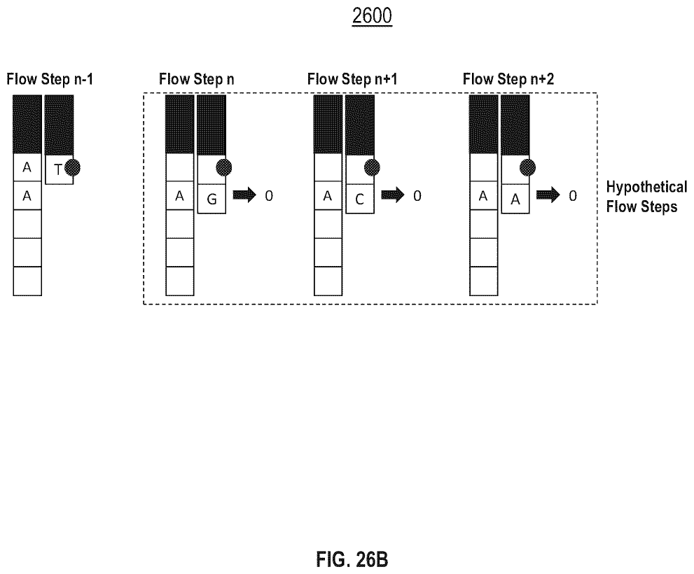

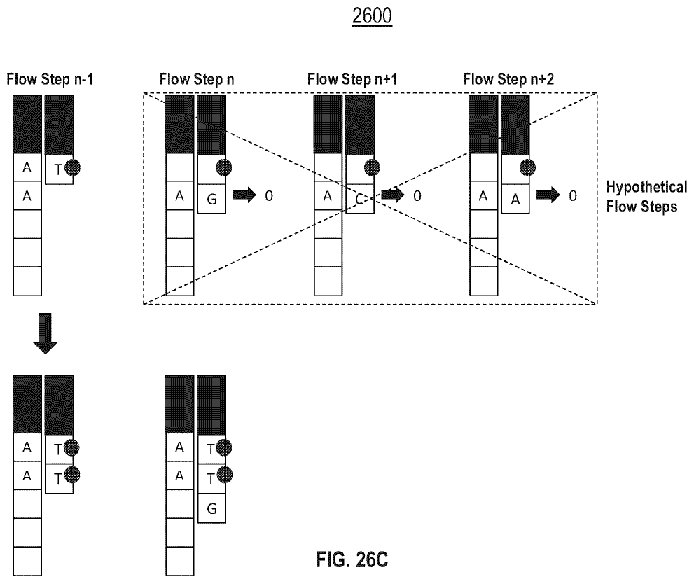

An absence of an incorporated nucleotide at three or more consecutive sequencing flow steps is indicative of weak, incorrect, or noisy signal(s) in the flow sequencing method, and thus an unreliable or damaged sequencing read. FIGS. 26A-26C illustrate an exemplary scenario demonstrating why an absence of an incorporated nucleotide at three consecutive sequencing flow steps cannot occur in a normal sequence. In the flow sequencing method 2600, the flow-cycle order is T-G-C-A. In some embodiments, the flow-cycle order is e.g., T-C-G-A, T-A-G-C, or any other permutation of the nucleotides T (or U), G, C, and A. Using an exemplary flow-cycle order T-G-C-A, in flow step n−1, labeled T nucleotides are combined with the hybrid; in flow step n, labeled G nucleotides are combined with the hybrid; in flow step n+1, labeled C nucleotides are combined with the hybrid; in flow step n+2, labeled A nucleotides are combined with the hybrid.

FIG. 26A depicts an impossible hypothetical scenario in which three consecutive sequencing flow steps, n to n+2, all yield a signal of 0 indicating an absence of an incorporated nucleotide. Specifically, in flow step n, labeled G nucleotides are not combined with the hybrid due to the A base; in flow step n+1, labeled C nucleotides are not combined with the hybrid due to the C base; in flow step n+2, labeled A nucleotides are not combined with the hybrid due to the A base.

For the hypothetical scenario in FIG. 26A to occur, there must be a nucleotide incorporation in step n−1 as shown by 2602. This is because if there is no nucleotide incorporation in step n−1, in step n, nucleotides G would be combined with the hybrid having the base before A, rather than the hybrid having the base A.

For nucleotide incorporation to occur in step n−1 where labeled T nucleotides are applied, it follows that the base before A in the template polynucleotide must be A (as the T base is complementary to the A base), as shown in FIG. 26B. However, if the base before A in the template polynucleotide is A, the hypothetical flow sequencing steps n to n+2 would not occur. Rather, as shown in FIG. 26C, when labeled T nucleotides are applied in step n−1, two T nucleotides are incorporated into the extending sequencing primer because the template sequence includes two consecutive A bases. Thus, the flow steps n to n+2 depicted in FIG. 26A would not occur normally.

Thus, FIGS. 26A-26C demonstrate why an absence of an incorporated nucleotide at three consecutive sequencing flow steps (a ‘3Z’ data pattern) cannot occur in a normal sequence. As shown in FIG. 23B, an absence of an incorporated nucleotide can occur in at most two consecutive sequencing flow steps. An absence of an incorporated nucleotide at three or more consecutive sequencing flow steps is indicative of weak, incorrect, or noisy signal(s) in the flow sequencing method, and thus an unreliable or damaged sequencing read. For example, it may indicate that there was a base in the template sequence that had been missed (e.g., indicative of degradation of the template sequence). Thus, any sequencing read having such absence is filtered in block 2404 such that the sequencing read is not used in downstream tasks (e.g., alignment to a reference genome or portions thereof, for SNP calling, etc.).

At block 2406, the system determines, by the one or more processors, for each flow step of each sequencing read, a read quality metric. For example, with reference to FIG. 23A, for each flow step (i.e., each column in the flow gram), a read quality metric (also known as regressed residual) is calculated. For example, for flow step 2302, a read quality metric RQM1 is calculated; for flow step 2306, RQM3 is calculated.

In some embodiments, the read quality metric for each flow step of each sequencing read is calculated based on a second highest homopolymer probability value (p_{2nd}). For example, in flow step 2302 in FIG. 23A, the second highest probably value is 0.0010. In some embodiments, the read quality metric is calculated as:

rₛ=log₁₀(p_{2nd}/ϵ)/10:

Where ϵ is a scaling factor and p_{2nd} is the second highest probability at the flow step (e.g., representing the second most likely h-mer). In some embodiments, E can be set at any value within the range 1×10⁻²-1×10⁻⁴.

The read quality metric for a given flow step can, in some instances, be calculated using other techniques. For example, in some embodiments, the value (1−p₁ₛₜ) can be used rather than p_{2nd} in the formula above. For instance, in cases in which p₁ₛₜ+p_{2nd}=1, the two formula variations would yield the same read quality metric. In cases in which p₁ₛₜ+p_{2nd}+p_{3rd}=1 (i.e., where probabilities are determined for more than two h-mers), these two example formula variations would yield different read quality metrics.

A higher read quality metric can be indicative of a weaker signal. For example, a higher value of read quality metric can indicate a lower p. Because the base count associated with p is selected, a lower p can indicate a lower confidence in the selected base count. Thus, the read quality metric is used to determine low confidence, which can indicate deterioration, in a sequencing read and determine where to trim the sequencing read, as described below.

At block 2408, the system trims the terminus of one or more sequencing reads in the sequencing data based on the read quality metrics for a respective sequencing read, thereby generating trimmed sequencing data. With reference to FIG. 25C, some of the sequencing reads 1−m are trimmed, thereby generating trimmed sequencing data.

In some embodiments, if a flow sequencing step produces a read quality metric below a predetermined threshold, the system can determine that deterioration has occurred in the sequencing read. Accordingly, the system can trim the sequencing read at or before the first flow sequencing step that produces a read quality metric below the threshold.

In some embodiments, the system uses an average of multiple read quality values to detect determination in the sequencing read. In some embodiments, the average is a moving average. Exemplary calculation of the moving average is described with reference to FIG. 23A. For example, at the third flow step, the system can calculate an average of RQM1, RQM2, and RQM3 (assuming the moving average is calculated using a sliding window of 3 flow steps); at the fourth flow step, the system can calculate an average of RQM2, RQM3, and RQM4. Thus, the moving average is a local quality measure.

In some embodiments, if the moving average exceeds a predetermined threshold, the system determines that deterioration has occurred and trims the sequencing read accordingly. In some embodiments, if a predefined number of moving averages are above the predetermined threshold, the system determines that deterioration has occurred. For example, the flow sequencing step that triggers trimming is the nth sequencing flow step having a moving average above a predetermined threshold, wherein n is a predefined number. The predetermined threshold can be a fixed value that can be tuned. For example, the predetermined threshold can be set to an average of the first 100 flow steps in a flow sequencing method. In some embodiments, the threshold is around 0.3.

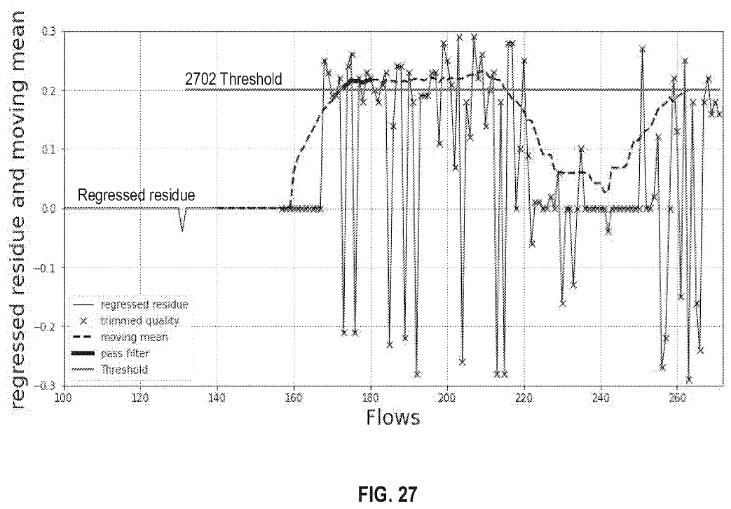

FIG. 27 illustrates the read quality metrics for an exemplary sequencing read, in accordance with some embodiments. In the depicted example, each cross indicates the read quality metric calculated at the corresponding flow step. The dashed line indicates the moving averages. The horizontal line 2702 indicates the predetermined threshold. If a predefined number of consecutive moving averages exceed the predetermined threshold (as shown by the bolded portion of the dashed line above the line 2702), the system determines that deterioration has occurred and therefore trims the sequencing read.

The system then trims at least the portion of the sequencing read comprising the selected sequencing flow step. In some embodiments, a predetermined number of consecutive sequencing flow steps prior to the selected sequencing flow step are also trimmed. In some embodiments, the predetermined number of consecutive sequencing flow steps is a multiple of four (e.g., 8 previous flow steps, 12 previous flow steps, 16 previous flow steps). In other words, the system also trims multiples of 4 flow steps before the selected flow step, in addition to trimming the selected flow step.

Thus, the trimming operation in block 2408 can be dependent on at least three parameters: window length, threshold, and lag. Window length refers to the size of the sliding window in which the moving average value is calculated. Threshold refers to the predetermined threshold of the moving average value above which the system determines that deterioration has occurred. Lag refers to the predetermined number of consecutive sequencing flow steps prior to the selected sequencing flow step that are also trimmed. In some embodiments, some or all of these parameters can be determined based on user input. In some embodiments, some or all of these parameters can be determined automatically.

In some embodiments, the system does not calculate a read quality metric for every flow step, but rather at regular intervals (e.g., every 4 flow steps, every 8 flow steps, etc.). In some embodiments, the system does not calculate read quality metrics for certain flow steps in a flow sequencing method (e.g., the first 100 flow sequencing steps), for example because deterioration typically occurs during later flow steps.

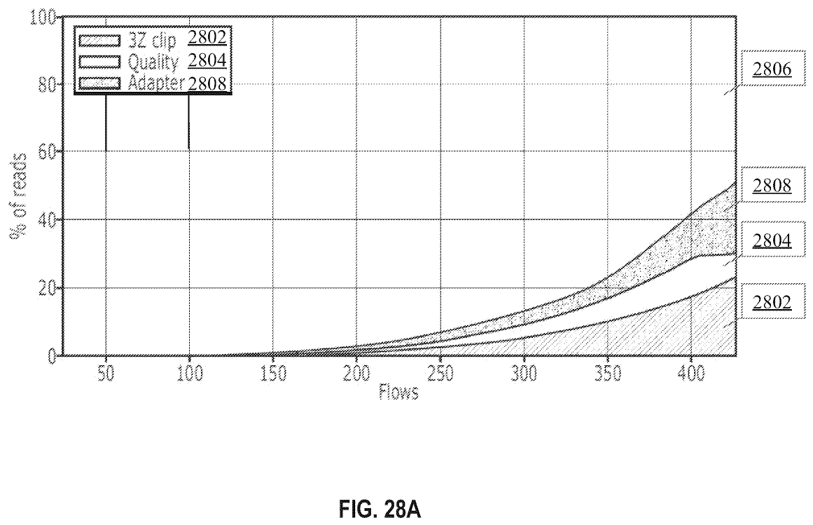

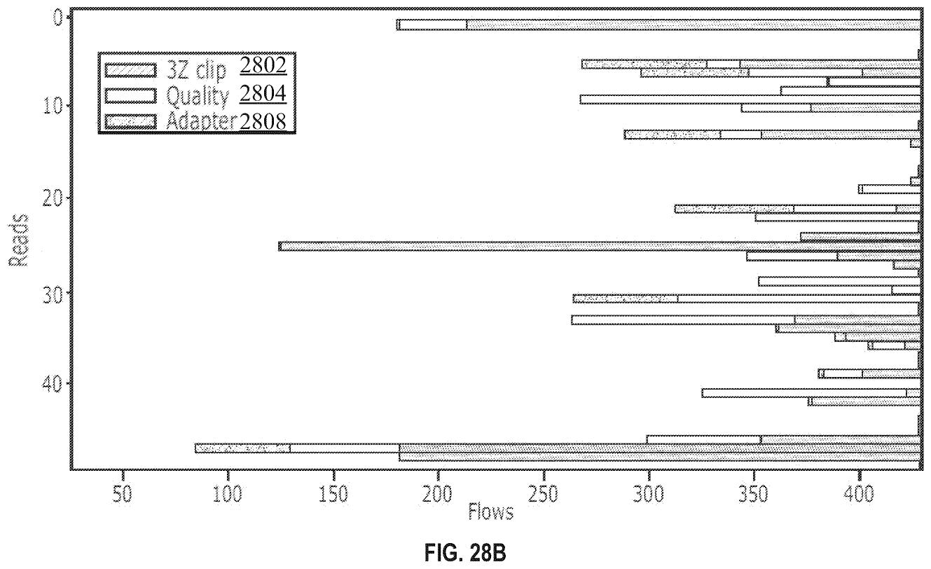

FIG. 28A illustrates that quality issues may occur to an increasing percentage of reads as the number of flows increases. As shown by the area 2802, as the number of flows increases, a higher percentage of sequencing reads are filtered (referred to as “3Z clip”) due to the absence of an incorporated nucleotide at three or more consecutive sequencing flow steps in these sequencing reads. As shown in the area 2804, as the number of flow steps increases, a higher percentage of sequencing reads are trimmed based on read quality metric calculations (referred to as “Quality”). For example, at flow step 350, about 10% of reads are removed and <10% of reads are trimmed. At flow step 400, about 30% of the sequencing reads have quality issues and are either trimmed or removed, and about 70% of the sequencing reads do not have quality issues (as shown in area 2806).

In addition, in the depicted example, the percentage of reads with trimmed adaptors 808 increases as the number of flow steps increases. This may be because the adaptor sequences are at the opposite end of reads from the primer where the sequencing begins. Thus, adaptor sequences are only observed (and then trimmed) in later flows. In FIG. 28B, the segments of the shading 2808 indicate reads that are trimmed due to adapter identification.

FIG. 28B illustrates 50 exemplary sequencing reads in accordance with some embodiments. The 50 sequencing reads are represented by 50 horizontal lines. Every line starts with a white segment, indicating that no quality issues have been detected. In some of the reads, quality issues are eventually detected. For example, in read 2808, around flow step 180, an absence of an incorporated nucleotide at three or more consecutive sequencing flow steps is detected. At around flow step 220, deterioration is detected based on read quality metrics. If the method 2400 in FIG. 24 is performed to process the 50 reads, any of the 50 reads that has an absence of an incorporated nucleotide at three or more consecutive sequencing flow steps (i.e., any read having the segment of the shading 2802) would be filtered in block 2404; any of the remaining reads that have deterioration based on the read quality metric (i.e., any read having a segment of the shading 2804 but not a segment of shading 2802) would be trimmed in block 2408. The segments of the shading 2808 indicate reads that are trimmed due to adapter identification. In some embodiments, the identification and trimming of adapters are performed after the initial trimming in block 2408.

In some embodiments, the system trims a known adapter sequence, or portion thereof, from one or more sequencing reads in the sequencing data. Sequencing adapters (e.g., adaptor 2201 in FIG. 22) can be ligated to the ends of the individual nucleic acids. The adapters serve as binding sites for primers (e.g., primer 2203 in FIG. 22). It can be beneficial to trim the adaptors because they can increase the file size (e.g., the CRAM file size) but are not useful for downstream tasks. Trimming the adaptors can improve data quality (e.g., for variant calling) while reducing the size of output files.

Reads that are trimmed in accordance with this example may be padded (e.g., with masking values as described elsewhere herein). Thus, these trimmed reads may, in some instances, be included in one or more downstream analyses.

In some embodiments, the system stores the trimmed sequencing data in a non-transitory computer readable medium.

In some embodiments, the system aligns sequencing reads in the trimmed sequencing data to a reference sequence (e.g., for variant calling). The method 2400 improves the quality of the sequencing reads (e.g., by removing undesirable reads and/or trimming undesirable portions of reads). The resulting sequencing reads are more likely to be aligned to the reference genome. In some embodiments, at least a predetermined percentage of sequencing reads in the trimmed sequencing data are aligned to the reference sequence. In some embodiments, the predetermined percentage is about 70%, about 75%, about 80%, about 85%, about 90%, about 95%, about 99%, or about 100%. In some embodiments, the system calls, using the one or more processors, one or more genetic variants using the trimmed sequencing data set. In some embodiments, the method 2400 is agnostic in terms of nucleotide data and thus can be used for RNA and/or DNA.

### Probability Neural Networks and Variant Calling

Provided herein are methods and systems for determining the likelihood of a particular nucleotide sequence from signals produced during a sequencing reaction (e.g., a sequencing by synthesis reaction, a flow chemistry reaction, or a reverse terminator reaction). The methods and systems may analyze the signals (e.g., an image, an intensity, a wavelength, or a position) to determine sequence information. For example, a method of the present disclosure may analyze a signal from a reverse-terminator chemistry sequencing method to determine an identity of a nucleotide base (e.g., A, T, C, or G). In another example, a method of the present disclosure may be used to analyze a signal from a flow-chemistry sequencing method to determine a number of nucleotide bases incorporated for a given flow (e.g., 0, 1, 2, 3, 4, or more A bases incorporated during an A flow, 0, 1, 2, 3, 4, or more T bases incorporated during a T flow, 0, 1, 2, 3, 4, or more C bases incorporated during a C flow, or 0, 1, 2, 3, 4, or more G bases incorporated during a G flow).

In some embodiments, the methods described herein may use a trained machine learning classifier (e.g., a neural network) to determine a probability that a given base (e.g., A, T, C, or G) was incorporated into a sequence or a given number of bases (e.g., 0, 1, 2, 3, 4, 5, 6, 7, 8, 9, 10, 11, 12, or more bases) was incorporated into a sequence based on a signal from a sequencing reaction. In some embodiments, the methods described herein may use a trained machine learning classifier (e.g., a neural network to determine a probability that a given signal was produced by incorporation of a certain base (e.g., A, T, C, or G) or a certain number of bases (e.g., 0, 1, 2, 3, 4, 5, 6, 7, 8, 9, 10, 11, 12, or more bases) during a sequencing reaction. The probability may be used to determine a confidence level or accuracy for a determined sequence. The neural network methods provided herein that output probabilities may provide an advantage over methods that output a most likely sequence because the probabilities may enable downstream bioinformatic analysis such as variant identification or variant calling analysis. While the process of translating the signals of a sequencing read (Rᵢ) into the corresponding most likely DNA sequence that generates it (haplotype, Hⱼ) may be used for bioinformatics analysis such as read alignment and RNAseq analysis, it may not fully meet requirements of the other downstream analysis, such as variant calling analysis.

A trained base calling Neural Network may be used in various method for analyzing the flow sequence signal to determine the probabilities for hmer prediction. The method may comprise: creation of ground truth and optimization of neural network model. To create ground truth, a set of reads may be used to train a model thereby producing a trained model. Then the reads may be aligned against a human genome, and only a selected subset of those reads which have good and unique alignment can be qualified to be used in a training set. In some embodiments, reads which do not meet a pre-determined criterion to qualify for use in a training set are filtered or discarded from use.

In some embodiments, the trained model may comprise a Convolutional neural network, which receives as input the measured signal and auxiliary field information, and outputs the expected genome key.

An optional procedure to derive the probability per reads may be using neural network that trained for optimizing the probability output. The Kullback—Leibler [KL] divergence theory measures the difference between two probability distributions.

$${D}_{KL} ( P ‖ Q ) = {\sum }_{\text{?}} P ( x ) log ( \frac{P ( x )}{Q ( x )} )$$

$$\text{?} \text{indicates text missing or illegible when filed}$$

In some embodiments, a base-calling method of the present disclosure may be implemented using a neural network. For example, a base-calling method may be implemented as follows:

$${\sum }_{i} {p}_{i} log {q}_{i}$$

The neural network may be optimized such that the KL divergence is reduce to the cross entropy loss function or other loss functions such as Hinge function, Huber function, MAE (LI) or MSE (K2):

Where p_i is the one-hot encoding vector of ground state probabilities, and q_i is the predicted probability vector. Training with the cross entropy loss function can produce a model that can predict the probability for a given hmer and flow given a measured read:

P(h|R)

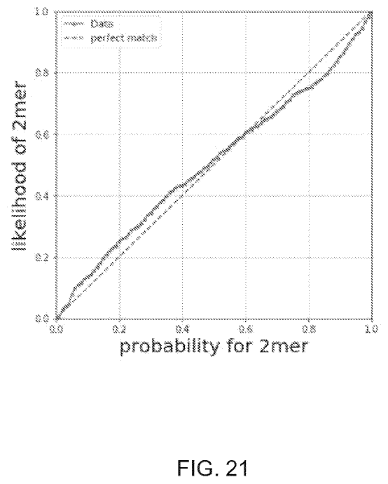

With h denoting a matrix of probabilities, its dimension is the number of flows x number of predicted probabilities. It can be verified that the probability output of the network is consistent with real probability in the following way: for each probability segment (for example between p=0.87 to p=0.88), accumulate all measurements that are predicted to be in this segment, and calculate an error rate for the data in this segment. If the percentage of corrected calls in this segment is 87%, this may be a verification for the accuracy of the probability prediction. FIG. 21 shows the relation between predicted probability and read correct call rate for 2mer data.

However, the above probability may not be the same probability required for variant calling. The probability that it is required as input for variant calling may be the Bayes-inverse probability to the one predicted in the model. This may be the probability of ‘measure read given a true key’ as used in equation (3)—listed below—by P(R|h).

The Bayes relation may be determined by the following equation:

P(R|h)=P(h|R)/P(h)

The latter equation means that to produce the probability necessary for the variant calling, the probabilities predicted by the neural network may be scaled. The scaling factor P(h) may be the probability for finding a certain hmer h in the entire genome and it can be calculated once using the distribution of hmers in the genome. This scaling may increase the probability of higher hmer compared to lower hmers to compensate for quantity difference between hmers populations.

Using the base calling results from the neural network methods described herein, variant calling may be performed. Variant calling may be used to determine, for each locus in the genome, the genotype probability based on multiple reads mapped to the locus P(Gᵢ|{R}). For example, for diploid genomes, the genotype probability is determined from each of the corresponding two haplotypes per locus, Gᵢ=H₁H₂.

The equations provided below may be used to determine the probability of sequence haplotype:

$$1. P ( {H}_{i} \vert \{ R \} ) = \frac{P ( \{ R \} \vert {H}_{t} ) P ( {H}_{t} )}{{Σ}_{k} P ( \{ R \} \vert {H}_{k} ) P ( {H}_{k} )} Bayesian statistics$$

$$2. P ( {H}_{i} \vert {R}_{j} ) = \frac{P ( {R}_{j} \vert {H}_{t} ) P ( {H}_{t} )}{{Σ}_{k} P ( {R}_{j} \vert {H}_{k} ) P ( {H}_{k} )} Bayesian statistics$$

$$3. P ( \{ R \} \vert {H}_{i} ) = {\prod }_{j} P ( {R}_{j} \vert {H}_{i} ) Reads independent$$

$$4. P ( {R}_{j} \vert {G}_{i} ) = \frac{1}{2} P ( {R}_{j} \vert {H}_{1} ) + \frac{1}{2} P ( {R}_{j} \vert {H}_{2} ) Haplotype independent$$

Combining equations (1), (3), and (4), a variant calling equation (5) (e.g., to determine P(Gᵢ|{R}) from P(Rⱼ|Hᵥ)) can be solved:

$$5. P ( {G}_{i} \vert \{ R \} ) ∝ P ( {G}_{i} ) {\prod }_{j} P ( {R}_{j} \vert {G}_{i} ) = P ( {G}_{i} ) {\prod }_{j} ( \frac{1}{2} P ( {R}_{j} \vert {H}_{1} ) + \frac{1}{2} P ( {R}_{j} \vert {H}_{2} ) )$$

In some embodiments, this equation may be implemented in a GATK tool, so that providing P(Rⱼ|Hᵥ) for all possible haplotypes of read j may enable clear integration with GATK and statistically solid solution for the variant calling problem.

The methods of this disclosure may provide advantages over use of likelihood algorithms to determine sequence variants. For example, likelihood algorithms (e.g., Pair Hidden Markov Models) may result in significant complexity, and inaccuracy may be attributed to the process of translating base calling standard output, P(Hⱼ|Rᵢ), into a P(Hⱼ|Hᵥ) for all possible haplotypes in the locus of read j. For example, in a GATK haplotype caller, this stage may be implemented by a Pair-HMM model that aims to capture the potential source of sequencing error and estimate the P(Rⱼ|Hᵥ).

The base calling methods provided herein (e.g., neural network base calling methods) may provide an improvement over likelihood-based variant calling algorithms. The base calling methods of this disclosure utilizing neural networks may provide information and method to estimate for each read j the likelihood, P(Rᵢ|Hᵥ), for all possible haplotypes, directly from a signal produced from a sequencing read. In some embodiments, a base calling method implementing a neural network may be optimized to UG flow-chemistry data to determine probabilities for all possible haplotypes.

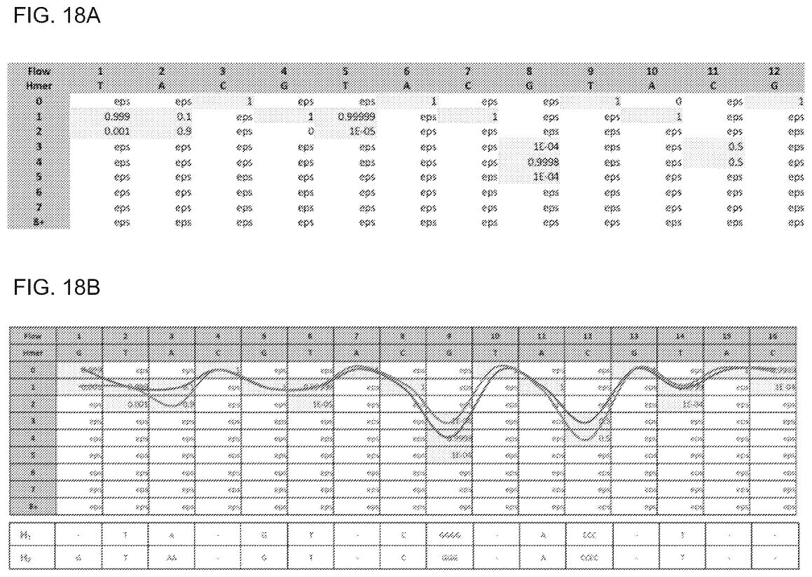

In some embodiments, the log likelihood of a given haplotype, P(Rⱼ|Hᵥ), may be determined from the output of the base calling methods described herein. For example, a matrix of probabilities may be generated based on signals produced from a flow-chemistry sequencing read. For each flow (e.g., a flow of A, T, C, or G nucleotides), the base calling methods provided herein may determine a probability of a given number of bases added during that flow (“hmer,” e.g., 0, 1, 2, 3, 4, 5, 6, 7, 8, 9, 10, 11, 12, or more bases added). The data may be output as a matrix of hmer versus flow number, representing the key space, for example as shown in FIG. 18A. Each flow number is associated with a corresponding nucleotide (e.g., A, T, C, or G) added during the flow. A sequence of a haplotype sequence in base space may be converted into probabilities in key space using a sequence flow order (e.g., TACG, ATCG, TAGC, ATGC, etc.). Haplotype key space sequence may be used to determine a path in the matrix (e.g., highlighted cells shown in FIG. 18A, or lines shown in FIG. 18B). Haplotype log likelihood may be determined by sum(log₁₀(P(haplotype path)). The most probable haplotype may be determined by (sum(log(max(P(h,f))).

In some embodiments, an output matrix may be large, leading to challenges when storing or sending the output matrices. To reduce the size of the data files, the matrices may be stored as sparse matrices. For example, cells with probability values below a threshold value may be set to a constant (e.g., epsilon, “eps,” shown in FIG. 18A). In some embodiments, cells representing significant alternative values (e.g., cells with the second highest probability for a given flow, shown in orange in FIG. 18A) may be reported in a FASTQ-like format.

The flow hmer probability matrix may be encoded in the FASTQ-like BAM format for compatibility with the existing tools. For example, the matrix may be encoded in the QUAL string field and an additional field ‘tip’. In some embodiments, only the probabilities for the flows where the hmer call is larger than zero are encoded.

The error probabilities may be encoded in the QUAL string which may show the probability of the error, and in the integer array tp tag which may show the difference between the error and the hmer call. The error may be encoded symmetrically relative to the middle of the hmer, the nucleotide on either side of the hmer half of the error probability. In some embodiments, up to min(4,floor((H+1)/2)) error probabilities may be reported, where H is the length of the most likely h-mer.

The error probabilities may be encoded in Phred format. For example, for a set of hmer probabilities P=(0,0,0,0.025,0.875,0.1,0), the hmer is called as H=4. The corresponding quality string that corresponds to the hmer may be: +11+, with the tp as +1,−1,−1,+1. In another example, the quality string corresponding to P=(0,0,0.025,0.875,0.1,0) may be +.+, while the tp is +1,−1,+1.

Methods and systems for improved base calling may use training sets for training a base caller (e.g., a trained machine learning classifier such as a neural network) which may be allowed to include reads of different lengths (e.g., thereby rescuing previously unusable reads). For example, a neural network that is trained for base caller may require input data of a fixed length in flow space (e.g., such that each read must include information for a same number of flows). Methods and systems of the present disclosure may comprise padding any “trimmed” reads with filler values (e.g., masked values), so that the training set may include a larger percentage of total reads. For example, masking values may be negative numbers, whereby different negative values encode for or indicate a different class of trimmed flows (e.g., flow quality, 3Z, adapters, errors, variants such as SNPs, etc.).

In some embodiments, a set of reads may be processed by trimming at least a subset of the reads, performing local alignment of at least a subset of the reads, performing adapter memorization of at least a portion of the reads, and analyzing initial flows. In some embodiments, one or more reads may be trimmed based at least in part on a “3Z” code (e.g., indicative of 3 consecutive 0-signal flows, as described above). For example, in a given read, the first flow with a “3Z” code and all later flows may be discarded from further consideration (e.g., for use in a base calling training set).

In some embodiments, reads may be trimmed based at least in part on a quality score. For example, all flows in a given read that fall below a pre-determined quality threshold may be discarded from further consideration (e.g., use in a base calling training set). In practice, this may result in all flows downstream of a quality drop being trimmed.

A quality score may be determined as follows. In its internal representation, each read may be encoded by an n_hmers×n_flows matrix, where a position (h, f) in the matrix describes a probability that the true flow corresponding to the read's flow f is h. This may be referred to as a “flow matrix”. Qual string and the true positive (TP) tag may encode the columns of the flow matrix for non-zero flows. Specifically, for hmer=H, we encode up to min(4,floor((H+1)/2)) error probabilities. QUAL encodes values of the probabilities, and TP encodes the value of the error relatively to the called hmer (e.g., error h=3 if the called hmer is 4 may be encoded as −1).

Probabilities in QUAL may be expressed using Phred-encoding. For convenience, the errors may be encoded symmetrically relative to the middle of the hmer, with the nucleotide on either side of the hmer capturing half of the error probability. As an example, for P=(0,0,0,0.025,0.875,0.1,0), the hmer called is H=4. QUAL is “+11+”. The tp is “+1,−1,−1,+1”. As another example, for P=(0,0,0.025,0.875,0.1,0), the hmer called is H=3. QUAL is “+.+”. The tp is “+1,−1,+1”.

In some embodiments, reads may be trimmed based at least in part on adapter trimming. For example, the adapter trimming may comprise removing or discarding any sequences that are recognized as an adapter sequence (e.g., a pre-determined adapter sequence).

The local alignment of reads may advantageously “rescue” some trimmed reads which would otherwise be discarded. In some embodiments, the local alignment of reads comprises adding masking values to reads for any flows that have been trimmed, thereby padding all reads to the same length. This local alignment approach may allow some mismatch for aligning, rather than requiring all aligned reads to have the same length. In some embodiments, the local alignment of reads is performed such that the largest segment of the read that is aligned predominates. In some embodiments, the local alignment of reads is performed such that the larger segment of the read that is aligned (e.g., Chimera reads) is selected and saved, with the remaining sequence masked. In some embodiments, the local alignment of reads is performed such that the if a middle portion of the read does not align, but the ends of the read do, then a read may be broken up into two sub-reads and separately aligned.

The local alignment of reads may advantageously serve as a replacement of Burrows-Wheeler alignment (BWA), which may be optimized for paired-end reads, with an aligner that functions in flow space (e.g., performing analog alignment of a set of flow signals to a set of reference flow signals) instead of base space (e.g., performing alignment of a string of nucleotide bases to a string of reference nucleotide bases). The flow-space aligner may have faster performance and/or improved variant calling as compared to a BWA aligner. In some embodiments, the flow-space aligner may be variant-aware (e.g., aligned such that a set of common variants is included). In some embodiments, the flow-space aligner may perform contamination detection (e.g., identify contamination from different genomes). In some embodiments, the flow-space aligner may feature re-defined mapping quality values (e.g., modified MapQ values for flow space).

The adapter memorization of reads may be performed in order to address issues with some reads being partially aligned while still including adapter sequences (e.g., such that the adapter sequence is mistakenly included as part of the genomic alignment). This can cause issues with incorrect alignment of sequence reads (e.g., even if 98% of adapter flows are identified, this can still cause issues downstream). In some embodiments, adapter memorization of reads may comprise inserting, in the sequence read data, an indicator of a set of pre-determined (e.g., known) adapter sequences. This, in some instances, may depend on having knowledge of the sequences and the locations of adapter sequences used in the sequencing run. For example, such adapters may be ligated onto one or both ends of nucleic acid molecules in order to facilitate nucleic acid sequencing (e.g., molecular barcoding, sample barcoding, etc.). By marking base calls or flows that are known to be part of an adapter sequence, these base calls or flows may be excluded from genome alignments.

In some instances, the initial flows may be analyzed. In some instances, an initial set of flows (e.g., the first 1, 2, 3, 4, or 5 flows) may be excluded from the training set due to uncertainty in calling the first base for the first h-mer of an insert.

### Computer Systems

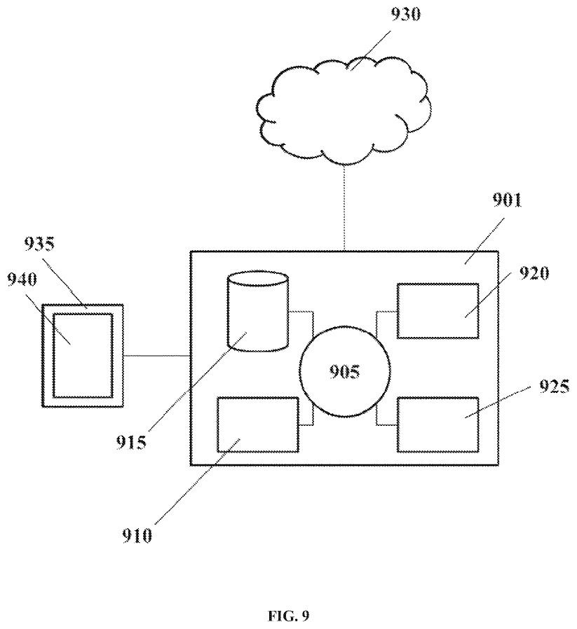

The present disclosure provides computer systems that are programmed to implement methods of the disclosure. FIG. 9 shows a computer system 901 that is programmed or otherwise configured to, for example, perform one or more operations of methods 100, 200, 300, 600, and 700. The computer system 901 can regulate various aspects of analysis, calculation, and generation of the present disclosure. The computer system 901 can be an electronic device of a user or a computer system that is remotely located with respect to the electronic device. The electronic device can be a mobile electronic device.

The computer system 901 includes a central processing unit (CPU, also “processor” and “computer processor” herein) 905, which can be a single core or multi core processor, or a plurality of processors for parallel processing. The computer system 901 also includes memory or memory location 910 (e.g., random-access memory, read-only memory, flash memory), electronic storage unit 915 (e.g., hard disk), communication interface 920 (e.g., network adapter) for communicating with one or more other systems, and peripheral devices 925, such as cache, other memory, data storage and/or electronic display adapters. The memory 910, storage unit 915, interface 920 and peripheral devices 925 are in communication with the CPU 905 through a communication bus (solid lines), such as a motherboard. The storage unit 915 can be a data storage unit (or data repository) for storing data. The computer system 901 can be operatively coupled to a computer network (“network”) 930 with the aid of the communication interface 920. The network 930 can be the Internet, an internet and/or extranet, or an intranet and/or extranet that is in communication with the Internet.

The network 930 in some cases is a telecommunication and/or data network. The network 930 can include one or more computer servers, which can enable distributed computing, such as cloud computing. For example, one or more computer servers may enable cloud computing over the network 930 (“the cloud”) to perform various aspects of analysis, calculation, and generation of the present disclosure, such as, for example, performing one or more operations of methods 100, 200, 300, 600, and 700. Such cloud computing may be provided by cloud computing platforms such as, for example, Amazon Web Services (AWS), Microsoft Azure, Google Cloud Platform, and IBM cloud. The network 930, in some cases with the aid of the computer system 901, can implement a peer-to-peer network, which may enable devices coupled to the computer system 901 to behave as a client or a server.

The CPU 905 may comprise one or more computer processors and/or one or more graphics processing units (GPUs). The CPU 905 can execute a sequence of machine-readable instructions, which can be embodied in a program or software. The instructions may be stored in a memory location, such as the memory 910. The instructions can be directed to the CPU 905, which can subsequently program or otherwise configure the CPU 905 to implement methods of the present disclosure. Examples of operations performed by the CPU 905 can include fetch, decode, execute, and writeback. The CPU 905 can be part of a circuit, such as an integrated circuit. One or more other components of the system 901 can be included in the circuit. In some cases, the circuit is an application specific integrated circuit (ASIC).

The storage unit 915 can store files, such as drivers, libraries and saved programs. The storage unit 915 can store user data, e.g., user preferences and user programs. The computer system 901 in some cases can include one or more additional data storage units that are external to the computer system 901, such as located on a remote server that is in communication with the computer system 901 through an intranet or the Internet.

The computer system 901 can communicate with one or more remote computer systems through the network 930. For instance, the computer system 901 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 901 via the network 930.

Methods as described herein can be implemented by way of machine (e.g., computer processor) executable code stored on an electronic storage location of the computer system 901, such as, for example, on the memory 910 or electronic storage unit 915. The machine executable or machine-readable code can be provided in the form of software. During use, the code can be executed by the processor 905. In some cases, the code can be retrieved from the storage unit 915 and stored on the memory 910 for ready access by the processor 905. In some situations, the electronic storage unit 915 can be precluded, and machine-executable instructions are stored on memory 910.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 901, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 901 can include or be in communication with an electronic display 935 that comprises a user interface (UI) 940 for providing, for example, a visual display indicative of sequencing signals, actual sequencing signals, accurate sequencing signals, etc. Examples of UIs include, without limitation, a graphical user interface (GUI) and web-based user interface.

Methods and systems of the present disclosure can be implemented by way of one or more algorithms. An algorithm can be implemented by way of software upon execution by the central processing unit 905. The algorithm can, for example, perform one or more operations of methods 100, 200, 300, 600, and 700.

### EXAMPLES

### Example 1

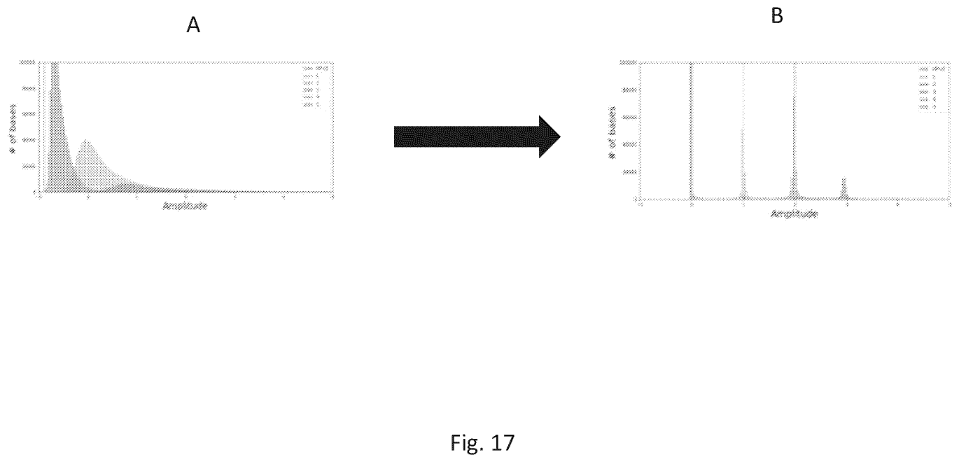

Using systems, methods, and media of the present disclosure, raw sequencing signals are generated from a plurality of nucleic acids. As shown in FIG. 17, a histogram is plotted of the number of bases of each of the raw sequencing signals having a given amplitude. A trained neural network is applied to the raw sequencing signals in order to identify and deconvolve systematics of the raw sequencing signals (such as phasing, signal decay, and context), shown in panel A, in order to generate processed sequencing signals (e.g., corrected or accurate sequencing signals), shown in panel B. A histogram of the processed signals (FIG. 17) shows narrow distributions of a number of bases of the processed sequences having amplitudes of about 0, 1, 2, and 3. The processed sequencing signals were produced without the use of a reference, thereby improving accuracy of sequence calling (e.g., sequences containing homopolymers).

### Example 2

Using systems, methods, and media of the present disclosure, a neural network is trained to produce a “ground truth” mapping between a plurality of input sequencing signals of a human or other large genome (e.g., generated from a plurality of nucleic acids) and a plurality of output sequences (e.g., comprising a plurality of base calls). First, base calling is performed on the plurality of input sequencing signals, thereby producing a plurality of initial sequences. This may be performed using a full base calling model (e.g., based on a large genome such as the human genome). The plurality of initial sequences may optionally be HpN-truncated, such that all homopolymers (e.g., of length, 2, 3, 4, . . . ) in the initial sequences are truncated to a length of 1 (e.g., represented by a single base) or another small number N, in order to ensure a low error rate of alignment. Next, the HpN-truncated sequences are aligned to a matching HpN-truncated human reference (e.g., the human genome that is HpN-truncated). Next, a training set is constructed using some or all of the HpN-aligned sequences (as outputs) and the associated sequencing signals (as inputs). Next, a neural network is trained using this training set, thereby producing a trained neural network.

Alternatively or in combination, at least a portion of the HpN-truncated sequences may be aligned to a matching E. coli (or other smaller genome) reference. A training set may be constructed using some or all of the HpN-aligned sequences (as outputs) and the associated sequencing signals (as inputs). A neural network may be trained using this training set, thereby producing a trained neural network. Existing models may be tested against the training set in order to select a model based on accuracy (e.g., the model that minimizes the base calling error).

### Example 3

Using systems, methods, and media of the present disclosure, a probability neural network is used to identify a base sequence of a polynucleotide and determine a probability and confidence value for the identified sequence. A nucleotide is sequenced using next generation sequencing (NGS) by synthesis methods in which a colony of identical DNA strands are synthesized in a controlled and synchronized matter such that a signal is generated upon incorporation of one or more bases. Sequencing may be performed using reverse terminator chemistry methods or flow chemistry methods. In the case of reverse terminator sequencing, sequencing methods are used to determine which base (e.g., A, C, T, or G) was incorporated into the DNA sequence. In the case of flow chemistry sequencing, the base calling methods of the present disclosure are used to determine how many of a given base are incorporated (e.g., when T nucleotides are flowed in, are 0, 1, 2, 3, 4, or more T bases added).

Sequencing base calling algorithms may output the most probable sequence per colony read based on the collected signals over sequencing flows and provide a quality score per base. The quality score may be indicative of the likelihood of error in a given reported base. The process of translating the signals of each read (which we define as Rᵢ) into the corresponding most likelihood DNA sequence that generates it (e.g., a haplotype, define as Hⱼ) may be useful for downstream bioinformatics analysis such as read alignment and RNAseq analysis. However, it may not fully meet requirements of the other downstream analysis, such as variant calling analysis.

The base calling methods of the present disclosure may use a probability neural network provide advantages over other sequencing algorithms. For example, multiple reads of a DNA sequence may be used to determine the likelihood that the observed signal is produced by a particular nucleic acid sequence. As another example, the base calling methods of the present disclosure may be implemented in combination with flow chemistry sequencing methods to determine the likely number of bases added per flow. The probability neural network base calling methods may analyze flow chemistry data and provide probability information to estimate the likelihood, P(Rⱼ|Hᵥ), of each possible haplotype from the raw signal of each read, j.

A trained base calling Neural Network may be used in various method for analyzing the flow sequence signal to determine the probabilities for hmer prediction. The method may comprise: creation of ground truth and optimization of neural network model. To create ground truth, a set of reads may be used to train a model thereby producing a trained model. Then the reads may be aligned against a human genome, and only a selected subset of those reads which have good and unique alignment can be qualified to be used in a training set. In some embodiments, reads which do not meet a pre-determined criterion to qualify for use in a training set are filtered or discarded from use.

In some embodiments, the trained model may comprise a Convolutional neural network, which receives as input the measured signal and auxiliary field information, and outputs the expected genome key.

An optional procedure to derive the probability per reads is using neural network that trained for optimizing the probability output. The Kullback-Leibler [KL] divergence theory measures the difference between two probability distributions.

$${D}_{KL} ( P ‖ Q ) = {\sum }_{\text{?}} P ( x ) log ( \frac{P ( x )}{Q ( x )} )$$

$$\text{?} \text{indicates text missing or illegible when filed}$$

Neural network-based sequence probability determination was performed as follows.

$${\sum }_{i} {p}_{i} log {q}_{i}$$

The KL divergence was reduced to the cross entropy loss function to optimize the neural network, where: pᵢ is the one-hot encoding vector of ground state probabilities, and qi is the predicted probability vector.

Training with the cross entropy loss function produced a model that could predict the probability for a given hmer and flow given a measured read: P(h|R).

Where h is a matrix of probabilities, the size of h (e.g., the dimensions) are the number of flows multiplied by the number of predicted probabilities.

The probability output of the network can be verified as consistent with real probability in the following way: for each probability segment (e.g., between p=0.87 to p=0.88), all measurements that are predicted to be in this segment are accumulated, and calculate an error rate for the data in this segment. If the percentage of corrected calls in this segment is 87%, this served as a verification for the accuracy of the probability prediction. FIG. 21 shows an example of the relation between predicted probability and the read correct call rate for h-mers of size 2.

However, the above probability was not the same probability required for variant calling. The probability that it is required as input for variant calling was the Bayes-inverse probability to the one predicted in the model. This was the probability of ‘measure read given a true key’ as used in equation (3) by

P(R|h)

The Bayes relation is given by

P(R|h)=P(h|R)/P(h)

The latter equation meant that to produce the probability necessary for the variant calling, one needed to scale the probabilities predicted by the neural network. The scaling factor P(h) was the probability for finding a certain hmer h in the entire genome and it can be calculated once using the distribution of hmers in the genome. This scaling increased the probability of higher hmer compared to lower hmers to compensate for quantity difference between hmers populations.

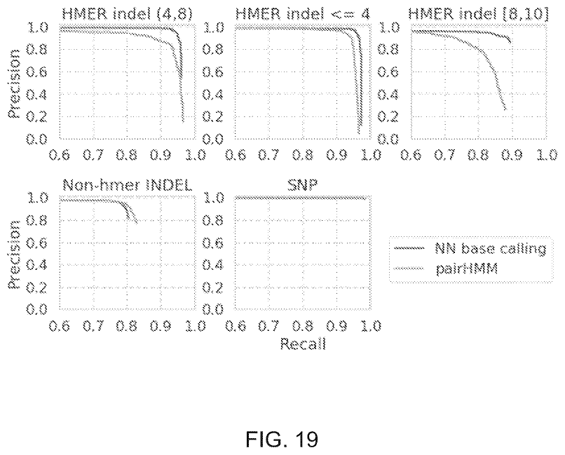

The neural network-based base calling coupled with the read-haplotype likelihood calculation by probability matrix haplotype scan methods of the present disclosure were compared to read-haplotype likelihood calculation by hidden Markov model (HMM) variant calling methods that utilized a standard GATK framework. A comparison of the neural network (“NN”) methods of the present disclosure to GATK framework HMM methods (“pairHMM”) are shown in FIG. 19. FIG. 19 shows a comparison of the precision and recall of each method for different types of sequences. The methods were compared for HMER insertion/deletions (“indel”) of various lengths (top three plots), non-hmer indels (bottom left), and single nucleotide polymorphisms (bottom right, “SNP”). The neural network methods of the present disclosure performed noticeably better for the HMER indels of various lengths.

A WGS library from a known sample (HG001/NA12878) was prepared and sequenced and variant calls from the modified HaplotypeCaller and from the original, PairHMM-based HaplotypeCaller were compared to the ground truth variants from that sample using GenotypeConcordance tool from picard tools. Variant calls were filtered by systematically testing different thresholds of the variant quality (QUAL) and strand bias metric (SOR) generating precision-recall curves that were compared (TABLE 2, FIG. 19).

TABLE 2

| | | GATK + NN | | Original GATK + no | |
|---|---|---|---|---|---|
| | Number | probability model | | probability calling | |
| Variant | of true | Optimal | Optimal | Optimal | Optimal |
| class | variants | precision | Recall | precision | Recall |
| SNV | 3042090 | 0.981 | 0.995 | 0.980 | 0.995 |
| Non-hmer | 218730 | 0.771 | 0.954 | 0.779 | 0.950 |
| INDEL | | | | | |
| HMER | 55393 | 0.956 | 0.932 | 0.923 | 0.951 |
| indel <= 4 | | | | | |
| HMER indel | 30506 | 0.956 | 0.954 | 0.871 | 0.931 |
| (4, 8) | | | | | |
| HMER indel | 52347 | 0.826 | 0.942 | 0.726 | 0.908 |
| [8, 10] | | | | | |

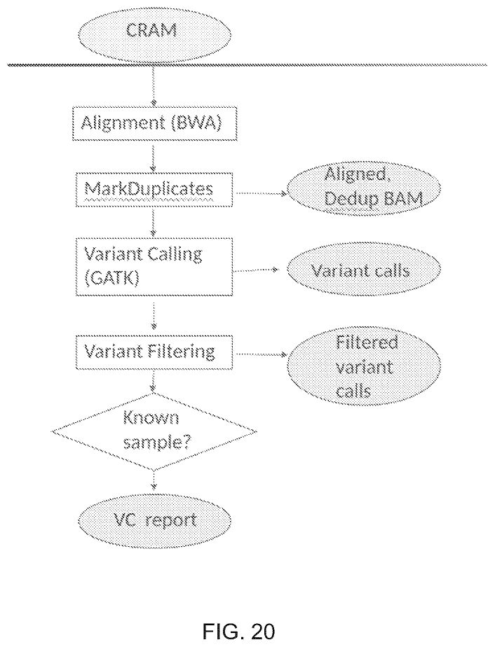

To implement the GATK framework, the GATK Haplotype caller tool called short variants from the aligned reads. First, for every genomic region of 500-1000 bases it generated a list of possible haplotypes by performing local de novo assembly on the reads that align to the region. Second, the probability P(Rⱼ|Hᵢ) for every read Rⱼ and proposed haplotype Hi was calculated, third, variants were called from the most likely haplotypes. In our implementation of HaplotypeCaller tool, the FASTQ-like format was converted back into the flow-hmer matrix per read. This matrix was then used to calculate P(Rⱼ|Hᵢ). The variants were then called and genotyped from IP(Rⱼ|Hᵢ)I_{i,j} by the same process as in the standard HaplotypeCaller. The processing pipeline for this method is shown in FIG. 20.

### Example 4

Using systems, methods, and media of the present disclosure, a nucleotide sequence variant and sample haplotype are identified, and the probability of the identified variant and haplotype are determined.

Multiple sequence reads of a DNA molecule with a sequence of TAAGTCGGGGACCC were performed using flow chemistry sequencing, and signals from the multiple reads are analyzed using the probability neural network methods of the present disclosure. The log likelihood of any potential haplotype, P(Rⱼ|Hᵥ), were calculated from the matrix using the following method:

The haplotype sequences were transformed from base space into key space using a flow order of the bases (TACGTACGTACG) shown at the top row of the table in FIG. 18A. The haplotype log likelihood was determined by sum(log₁₀(P(haplotype path)), and the most probable haplotype and its likelihood was determined by (sum(log(max(P(h,f))). The most probable haplotype was determined to be TAAGTCGGGGACCC, shown by the yellow cells in FIG. 18A. The log₁₀ likelihood of the probable haplotype was −0.35. The second most likely hmers are shown by the orange cells. Note that the likelihood of any cycle-shift from the most probable read path is practically zero. An independent flow model was assumed.

The flow hmer probability matrix was encoded in the FASTQ-like BAM format for compatibility with the existing tools. The matrix was encoded in the QUAL string field and an additional field that we call tp. Only the probabilities for the flows where the hmer call is larger than zero were encoded.

The error probabilities were encoded in the QUAL string which show the probability of the error, and in the integer array tp tag which show the difference between the error and the hmer call. The error was encoded symmetrically relative to the middle of the hmer, the nucleotide on either side of the hmer half of the error probability. Up to min(4,floor((H+1)₁₂)) error probabilities was reported.

The error probabilities were encoded in Phred format. For example, P=(0,0,0,0.025,0.875,0.1,0) and the hmer called was H=4. Quality string that corresponds to the hmer was: +11+; tp is +1,−1,−1,+1. For example: Quality string corresponding to P=(0,0,0.025,0.875,0.1,0) was +.+; tp is +1,−1,+1.

FIG. 18A shows the matrix output providing the probability that a given number of bases (“hmer”) were added during the specified nucleotide flow. The cells highlighted in yellow indicate the most probable. To reduce the data storage size, data was stored as a sparse matrix in which all cells with probability below a threshold were set to a constant (epsilon, “eps”). All significant alternative (Orange cells) are reported in the following “FAST-like” manner. FIG. 18B shows a second matrix providing the probability that a given number of bases (“hmer”) were added during the specified nucleotide flow. The most likely paths, representing the most likely haplotypes, are indicated by lines.

### Example 5

In some embodiments, barcode sequences may be generated and selected to provide a set of known barcodes. In some embodiments, the barcode sequences are generated based on one or more criteria and further selected by performing one or more filtering processes. This results in a set of barcodes with known sequence that fulfil one or more predetermined criteria.

Described herein is an example plan for barcode generation for use in sequencing applications. One such application is to be able to identify flows of interest from photometry data (e.g., just from the signals—such as optical signals—generated during sequencing), instead of after sequencing (e.g., after base calling). The results of sequencing a plurality of nucleic acid molecules, optionally comprising barcode sequences, may be output, e.g., using a processor, as information in flow space (e.g., a matrix or vector of flow data), which may be processed, prior to analysis by a neural network (i.e., for base calling).

In some embodiments, a set of sequencing signals and/or sequencing reads is analyzed to determine one or more sets of barcodes associated therewith. For example, a given barcode may be used to cluster a set of sequencing signals and/or sequencing reads by sample (e.g., in runs where there are multiple samples analyzed in parallel). In some embodiments, such barcode clustering may be performed on a set of sequencing signals and/or sequencing reads prior to performing read trimming.

Whole genome sequence (WGS) runs (e.g., genomic sequencing) may be distinguished from other applications such as RNA sequencing runs (or targeted sequencing, etc.), which are referred to herein as non-WGS runs.

The flow sequence used in these examples is TGCA. In some embodiments, the flow sequence may be any other permutation of the nucleotides T or U, G, C, and A (e.g., GTAC, ACTG, etc.).

Non-WGS runs: for non-WGS runs, the sequences for which a neural network model cannot be created may be measured. For such runs, a spike-in training data set may be added and used for creating the model. That training set may be labeled as described below to prevent contamination with the other data.

Training Set: The training data set that maybe used for training a model may comprise: a set of −100 million reads, comprising −80 million standard human reads and −20 million E. coli reads.

The training data may be identified by a training data indication barcode sequence that can be identified in one flow cycle (e.g., comprising one nucleotide base type). In some instances, the training data indication barcode is a sequence of TT (e.g., a sequence that results in a double addition of a nucleotide). That flow cycle will follow one flow sequence preamble (e.g., one iteration of T, G, C, A). In base space (e.g., nucleotide sequence), the template nucleic acid molecules may have a sequence of:
T, G, C, A, T, T, . . . Human Insert
In flow space, this preamble sequence and template data identification barcode may result in flow signals:
1, 1, 1, 1, 2, . . . Insert flows
Flows 0-3 may be the preamble flows (e.g., T, G, C, A, where the indexing begins at 0), setting the preamble as the flow order sequence in a single cycle. Flow 4 (e.g., flow cycle T) comprises the double TT; this may be used as a signal distinction between the training set and all other reads. Flows 5-7 may be uncertain: in determining the resulting sequence from the flow space results those flows cycles may not be presumed to be known and may not be a part of the training. In some embodiments, model training can start at the T flow cycle 8 (e.g., after the barcode and any uncertain intermediate sequence).
Other sequences
The sequences in the run barcodes (e.g., the test or sample sequences) will always start with a C:
T, G, C, A, C, . . . Insert of Interest . . .
In flow space may read as:
1, 1, 1, 1, 0, 0,1, . . . Insert flows

In this way, contamination of training data may be prevented at two steps: (i) training data may be identified by a distinct signal at flow 4, where training data signal is ‘2’ or greater and other signals are ‘0’. The strong signal separation between 2-mers and 0-mers prevents most mis-identifications.

Identification of barcodes can also include comparison of flows 4 and 5, which are always 0, 1.

Identification in photometry: The time-consuming process of identifying ˜100 million training reads in a substrate comprising 4 billion or more sequence reads may be avoided by identifying the training reads during photometry (e.g., during sequencing by synthesis using detection of identifiable signals during each flow cycle). During photometry, a sample data set, used for training may be copied to the monitoring computer system. Beneficially, instead of selecting the sample set randomly or after sequence, the training set may be identified at flow 4 via photometry (e.g., in flow space).

WGS run: For WGS runs the training is performed with a random set of data, where all the reads include barcode sequences. The training is performed on flows proceeding the barcodes, and the barcodes are used as analog correlations.

Example requirements for barcodes selections: In some instances, barcodes may be kept at a constant length in flow space (e.g., can be fully sequenced in the same number of flows, and requiring the same number of flows to be fully sequenced). In some instances, barcodes may be an edit distance of at least 2 from one another (e.g., as measured in vector space representing flow signals). In some instances, each of the values in flow space will be 0 or 1 (e.g., there will be no homopolymers in base space greater than 1). In some instances, the edit distance between barcodes may be based on 0-mers to 1-mers. In some instances, a minimum number of barcodes are required (e.g., at least 96×2 different barcodes). In some instances, barcodes in base space may be kept at similar (e.g., not exact) length. Additionally, in some instances, all barcodes may start with a single C. In some instances, all barcodes may start with a single nucleotide of a same type. For example, in all instances, all barcodes may start with a single A, all barcodes may start with a single T (or a U), or all barcodes may start with a single G. In some instances, flows for preamble and barcodes all start with the sequence 1,1,1,1,0,0,1 (e.g., in flow space). In some instances, starting with this sequence obviates reliance on the uncertain flows (e.g., flows 5-7 as described above) for barcode identification. In some instances, all barcodes end with a constant sequence to support un-biased library prep. In some instances, the constant sequence is GAT. In some instances, the last T (e.g., in the GAT constant ending sequence) of the barcode can be interpreted as part of the proceeding sequence, thus reducing the length of the called H-mer by ‘1’. In some instances, the constant sequence is any series of three nucleotides. In some instances, the constant sequence is a series of more than 3 nucleotides (e.g., 4 or more nucleotides, 5 or more nucleotides, etc.).

Barcodes: In some instances, with the above described restrictions, 16 flows may be used to arrive at a set of 238 barcodes. In such an instance, of those 16 flows, 7 flows are constant (e.g., 3 flows at the start of the barcode sequence and 4 flows at the end of the barcode sequence) and 9 flows (e.g., the middle flows) are variable. In such an instance, these barcodes will have either 9 or 11 bases (e.g., the barcodes are variable length in base space). Table 3 illustrates an example of barcodes from a set of 238 barcode sequences and the resultant flow space (e.g., vector of flow cycle values) for each such barcode sequence.

TABLE 3
List of example barcode sequences and the flow cycle values resulting from 20 flow
cycles, where the edit distance between each possible pair of barcode sequences is at least 2.

| Barcode | | | | | | | | | | | | | | | | | |
|---|---|---|---|---|---|---|---|---|---|---|---|---|---|---|---|---|---|
| \| Flows | 4T | 5G | 6C | 7A | 8T | 9G | 10C | 11A | 12T | 13G | 14C | 15A | 16T | 17G | 18C | 19A | 20T |
| CGTCA | 0 | 0 | 1 | 0 | 0 | 1 | 0 | 0 | 1 | 0 | 1 | 1 | 1 | 1 | 0 | 1 | 1 |
| TGAT | | | | | | | | | | | | | | | | | |
| CGTGA | 0 | 0 | 1 | 0 | 0 | 1 | 0 | 0 | 1 | 1 | 0 | 1 | 1 | 1 | 0 | 1 | 1 |
| TGAT | | | | | | | | | | | | | | | | | |
| CGTGC | 0 | 0 | 1 | 0 | 0 | 1 | 0 | 0 | 1 | 1 | 1 | 0 | 1 | 1 | 0 | 1 | 1 |
| TGAT | | | | | | | | | | | | | | | | | |
| CGTGC | 0 | 0 | 1 | 0 | 0 | 1 | 0 | 0 | 1 | 1 | 1 | 1 | 0 | 1 | 0 | 1 | 1 |
| AGAT | | | | | | | | | | | | | | | | | |
| CGACA | 0 | 0 | 1 | 0 | 0 | 1 | 0 | 1 | 0 | 0 | 1 | 1 | 1 | 1 | 0 | 1 | 1 |
| TGAT | | | | | | | | | | | | | | | | | |
| CGAGA | 0 | 0 | 1 | 0 | 0 | 1 | 0 | 1 | 0 | 1 | 0 | 1 | 1 | 1 | 0 | 1 | 1 |
| TGAT | | | | | | | | | | | | | | | | | |
| CGAGC | 0 | 0 | 1 | 0 | 0 | 1 | 0 | 1 | 0 | 1 | 1 | 0 | 1 | 1 | 0 | 1 | 1 |
| TGAT | | | | | | | | | | | | | | | | | |
| CGAGC | 0 | 0 | 1 | 0 | 0 | 1 | 0 | 1 | 0 | 1 | 1 | 1 | 0 | 1 | 0 | 1 | 1 |
| AGAT | | | | | | | | | | | | | | | | | |
| CGATA | 0 | 0 | 1 | 0 | 0 | 1 | 0 | 1 | 1 | 0 | 0 | 1 | 1 | 1 | 0 | 1 | 1 |
| TGAT | | | | | | | | | | | | | | | | | |
| CGATC | 0 | 0 | 1 | 0 | 0 | 1 | 0 | 1 | 1 | 0 | 1 | 0 | 1 | 1 | 0 | 1 | 1 |
| TGAT | | | | | | | | | | | | | | | | | |

Generating a larger number of barcodes (e.g., more than 238 barcodes) may require an increase in the acceptable barcode length in base space, and hence in flow space (e.g., as shown in FIG. 16). In generating a larger barcode set, it may also be beneficial to improve distinction among barcode sequences by increasing the effective edit-distance between each pair of barcode (e.g., from the minimum edit distance of 2 in Example 1 to a minimum edit distance of at least 4). In some embodiments, the effective-edit distance is at least 4, at least 5, at least 6, at least 7, at least 8, at least 9, at least 10, at least 11, at least 12, at least 13, at least 14, or at least 15.

In some instances, the requirements for generating a larger barcode set may include the following. In some instances, barcodes will have an effective edit distance of at least 4 from each other (e.g., there will be an edit distance of at least 4 between each possible pair of barcodes in the set). Specifically, in some instances, each of the values in flow space will be 0, 1, or 2 (e.g., there will be no homopolymers that are longer than 2 nucleotides long in base space). In some instances, only one value in flow space will be 2. In addition, in some instances, for each pair of barcodes most of substitutions between the vectors representing the barcodes (see e.g., Table 2 below) will be from a 0 to a 1 or a 1 to a 0, while few of the substitutions will be from a 1 to a 2 or from a 2 to a 1. Barcodes will have a constant length in flow space (as described above for Example 1). These parameters serve to increase the contribution of context to signal difference. In some instances, at least 1000 different barcodes are required. The constant length in flow space will lead to each of the barcodes having similar (but not exact) length in base space.

### Example 6

Current methods of base calling may face significant challenges, such as cases in which a significant amount of reads cannot be used for training a base caller. Such challenges may arise because the base caller training may require as inputs a set of fully aligned reads that are of the same length. Any reads that have quality issues and have to be ‘trimmed’ may be thus excluded from the training set. However, such exclusion may introduce undesirable bias into the training set toward long reads, which may be low copy, and can result in overfitting of the trained base caller.

Using methods and systems for improved base calling of the present disclosure, training sets used for training a base caller (e.g., a trained machine learning classifier such as a neural network) are allowed to include reads of different lengths (e.g., thereby rescuing previously unusable reads). For example, a neural network that is trained for base calling may require input data of a fixed length in flow space (e.g., such that each read must include information for a same number of flows). This is, in some embodiments, a requirement of the neural network (or of another machine learning model as described herein). The methods and systems for improved base calling of the present disclosure may comprise padding any “trimmed” reads with filler values (e.g., masked values). The masking values are denoted by negative numbers, whereby different negative values encode for or indicate a different class of trimmed flows (e.g., flow quality, 3Z, adapters, errors, variants, etc.). Advantageously, padding trimmed reads makes more reads eligible for incorporation into the training set, and thus training sets may include a larger percentage of total reads. Masking values are not included in downstream analysis. Instead, the use of masking values ensures that one or more trimmed reads retain the apparent length of untrimmed reads (e.g., in flowspace).

A set of reads may be processed by one or more of: trimming at least a subset of the reads, performing local alignment of at least a subset of the reads, performing adapter identification of at least a portion of the reads, and analyzing initial flows. In some instances, one or more of these processes may be applied to an individual read.

Some reads are trimmed based at least in part on a “3Z” code (e.g., indicative of 3 consecutive 0-signal flows). For example, in a given read, flows included in a “3Z” code and all later flows in the read are discarded from further consideration (e.g., discarded from a base calling training set). Some reads are trimmed based at least in part on a quality score. For example, all flows in a given read that fall below a pre-determined quality threshold are discarded from further consideration (e.g., use in a base calling training set). In practice, this results in all flows downstream of a quality drop being trimmed.

A quality score is determined as follows. In its internal representation, each read is encoded by an n_hmers×n_flows matrix, where a position (h, f) in the matrix describes a probability that the true flow corresponding to the read's flow f is h. This is referred to as a “flow matrix”. Qual string and the true positive (TP) tag encode the columns of the flow matrix for non-zero flows. Specifically, for hmer=H, we encode up to min(4,floor((H+1)/2)) error probabilities. QUAL encodes values of the probabilities, and TP encodes the value of the error relatively to the called hmer (e.g., error h=3 if the called hmer is 4 may be encoded as −1).

Probabilities in QUAL are expressed using Phred-encoding. For convenience, the errors are encoded symmetrically relative to the middle of the hmer, with the nucleotide on either side of the hmer capturing half of the error probability. As an example, for P=(0,0,0,0.025,0.875,0.1,0), the hmer called is H=4. QUAL is “+11+”. The tp is “+1,−1,−1,+1”. As another example, for P=(0,0,0.025,0.875,0.1,0), the hmer called is H=3. QUAL is “+.+”. The tp is “+1,−1,+1”.

Some reads are trimmed based at least in part on adapter trimming. For example, the adapter trimming comprises removing or discarding any sequences that are recognized as an adapter sequence (e.g., a pre-determined adapter sequence). In some embodiments, adaptor sequences may be identified through adaptor memorization, as described elsewhere herein.

Performing local alignment of reads advantageously “rescues” some trimmed reads which would otherwise be discarded. The local alignment of reads comprises adding masking values to reads for any flows that have been trimmed, thereby padding all reads to the same length. This local alignment approach allows for some mismatch for aligning, rather than requiring all aligned reads to have the same length. The local alignment of reads is performed such that the largest segment of the read that is aligned predominates. The local alignment of reads is performed such that the larger segment of the read that is aligned (e.g., Chimera reads) is selected and saved, with the remaining sequence masked. The local alignment of reads is performed such that the if a middle portion of the read does not align, but the ends of the read do, then a read is broken up into two sub-reads and separately aligned.

The local alignment of reads advantageously serves as a replacement of Burrows-Wheeler alignment (BWA), which is optimized for paired-end reads, with an aligner that functions in flow space (e.g., performing analog alignment of a set of flow signals to a set of reference flow signals) instead of base space (e.g., performing alignment of a string of nucleotide bases to a string of reference nucleotide bases). The flow-space aligner has faster performance and/or improved variant calling as compared to a BWA aligner. The flow-space aligner is variant-aware (e.g., aligned such that a set of common variants is included). The flow-space aligner performs contamination detection (e.g., identify contamination from different genomes). The flow-space aligner features re-defined mapping quality values (e.g., modified MapQ values for flow space).

In some embodiments, adapter memorization of reads is performed in order to address issues with some reads being partially aligned while still including adapter sequences (e.g., such that the adapter sequence is mistakenly included as part of the genomic alignment), which makes it difficult to identify all adapter flows (e.g., even if 98% of adapter flows are identified, this can still cause issues downstream). Adapter memorization of reads comprises manually inserting an indicator of a set of pre-determined (e.g., known) adapter sequences, which may depend on having knowledge of the adapter sequences used. For example, such adapters are ligated onto one or both ends of nucleic acid molecules in order to facilitate nucleic acid sequencing (e.g., molecular barcoding, sample barcoding, etc.).

In some embodiments, initial flows of one or more sequence reads are analyzed. In some instances, an initial set of flows (e.g., the first 1, 2, 3, 4, or 5 flows) are instead excluded from the training set due to uncertainty in calling the first base for the first h-mer of an insert.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A method for determining a sequence of a nucleic acid, comprising:
(a) receiving a plurality of sequencing signals of the nucleic acid that are generated at least in part by imaging a substrate comprising a plurality of substrate segments;
(b) applying a trained algorithm to at least a portion of the plurality of sequencing signals to estimate a likelihood that one or more of the plurality of sequencing signals is produced by a particular nucleic acid sequence; and
(c) determining the sequence of the nucleic acid based at least in part on the estimated likelihoods from (b).

2. The method of claim 1, wherein the nucleic acid comprises deoxyribonucleic acid (DNA) or ribonucleic acid (RNA).

3. The method of claim 1, wherein the plurality of sequencing signals is generated at least in part by performing flow sequencing of the nucleic acid.

4. The method of claim 3, wherein the plurality of sequencing signals comprises analog values produced by the imaging.

5. The method of claim 4, wherein the analog values comprise fluorescence signals.

6. The method of claim 5, wherein the fluorescence signals correspond to discrete DNA extensions sensed from introduction of single nucleotide solutions in the flow sequencing.

7. The method of claim 6, wherein the introduction of single nucleotide solutions in the flow sequencing is cyclic.

8. The method of claim 6, wherein the introduction of single nucleotide solutions in the flow sequencing is acyclic.

9-11. (canceled)

12. The method of claim 1, wherein the plurality of substrate segments comprises a same shape and/or size.

13. The method of claim 1, wherein at least two of the plurality of substrate segments differ by at least one shape and size.

14. The method of claim 1, wherein (b) further comprises estimating a likelihood of each of a plurality of haplotypes, and wherein (c) further comprises determining the sequence of the nucleic acid based at least in part on the estimated likelihoods of each of the plurality of haplotypes.

15-18. (canceled)

19. The method of claim 1, wherein the trained algorithm is trained at least in part by: obtaining a training set comprising a plurality of training sequencing signals and a plurality of training sequencing reads associated therewith, and using the training set to generate the trained algorithm, wherein the trained algorithm comprises a mapping between input sequencing signals and output sequencing reads comprising base calls.

20. The method of claim 19, wherein the training sequencing reads in the plurality of training sequencing reads are aligned to a reference genome.

21. The method of claim 20, wherein the aligning is performed in flow space.

22. The method of claim 20, wherein the aligning comprises: (i) using a set of common base calling variants, (ii) detecting contamination from a different genome, or (iii) using indicators of pre-determined adapter sequences.

23.-24. (canceled)

25. The method of claim 20, wherein the plurality of training sequencing reads is filtered to remove at least one training sequencing read that: (i) is not fully aligned to the reference, (ii) does not comprise a largest segment that is fully aligned to the reference, (iii) has a quality score that fails to meet a pre-determined criterion, (iv) has a length that differs from a reference length, or (v) comprises a pre-determined adapter sequence.

26-30. (canceled)

31. The method of claim 19, wherein at least one of the training sequence reads in the plurality of training sequencing reads is padded with filler values, such that the plurality of training sequencing reads has a substantially identical length.

32. The method of claim 31, wherein the filler values are masking values comprising negative numbers, and are indicative of a class of trimmed flows, wherein the class of trimmed flows is selected from the group consisting of low quality flows, flows comprising three consecutive zero-signals, flows with errors, and flows with variants.

33-34. (canceled)

35. The method of claim 1, further comprising determining a likelihood of the sequence of the nucleic acid determined in (c) being correct.

36. The method of claim 1, further comprising determining a maximum likelihood h-mer length of the sequence of the nucleic acid.

37-97. (canceled)


SEQUENCE LISTING (4 sequences)

SEQ ID NO: 1 - DNA, 12 bp, Artificial Sequence
  Synthetic oligonucleotide
tatggtcgtcga

SEQ ID NO: 2 - DNA, 14 bp, Artificial Sequence
  Synthetic oligonucleotide
taagtcggggaccc

SEQ ID NO: 3 - DNA, 14 bp, Artificial Sequence
  Synthetic oligonucleotide
tagtcggggaccct

SEQ ID NO: 4 - DNA, 16 bp, Artificial Sequence
  Synthetic oligonucleotide
gtaagtcgggacccct